(19) United States
(12) Patent Application Publication
(10) Pub. No.: US 2019/0216779 A1
(43) Pub. Date: Jul. 18, 2019

(54) USE OF NEUROKININ-1 ANTAGONISTS TO TREAT A VARIETY OF PRURITIC CONDITIONS

(71) Applicant: MENLO THERAPEUTICS INC., Redwood City, CA (US)

(72) Inventors: Steven BASTA, Menlo Park, CA (US); Mark JOING, Palo Alto, CA (US); Xiaoming ZHANG, Sunnyvale, CA (US); Paul KWON, Danville, CA (US)

(73) Assignee: Menlo Therapeutics Inc., Redwood City, CA (US)

(21) Appl. No.: 16/312,900

(22) PCT Filed: Jun. 28, 2017
(86) PCT No.: PCT/US2017/039829

§ 371 Date: Dec. 21, 2018

Related U.S. Application Data
(60) Provisional application No. 62/356,301, filed on Jun. 29, 2016.
(60) Provisional application No. 62/356,294, filed on Jun. 29, 2016.
(60) Provisional application No. 62/356,291, filed on Jun. 29, 2016.
(60) Provisional application No. 62/356,286, filed on Jun. 29, 2016.
(60) Provisional application No. 62/356,280, filed on Jun. 29, 2016.
(60) Provisional application No. 62/356,271, filed on Jun. 29, 2016.
(60) Provisional application No. 62/356,264, filed on Jun. 29, 2016.

(51) Int. Cl. A61K 31/4035; A61P 17/04; A61K 9/00; A61K 9/20; A61P 17/06; A61P 35/00

(52) U.S. Cl. CPC A61K 31/4035; A61P 17/04; A61K 9/0053; A61K 45/06; A61P 17/06; A61P 35/00; A61K 9/20

(57) ABSTRACT

The disclosure relates to the use of neurokinin-1 (NK-1) antagonists, such as serlopitant, in treating acute or chronic pruritus associated with a variety of medical conditions, including dermatitis/eczema, psoriasis, prurigo, urticaria, cutaneous T-cell lymphoma, epidermolysis bullosa, bums and hepato-biliary diseases, or/and treating the medical conditions themselves. One or more additional antipruritic or therapeutic agents can optionally be used in combination with an NK-1 antagonist to treat acute or chronic pruritus associated with a medical condition or/and the medical condition itself.

### CROSS REFERENCE TO RELATED APPLICATIONS

This application claims priority to and the benefit of U.S. Provisional Application Nos. 62/356,264; 62/356,271; 62/356,280; 62/356,286; 62/356,291; 62/356,294 and 62/356,301, each of which was filed on Jun. 29, 2016, and the entire disclosure of each of which is incorporated herein by reference for all purposes.

### TECHNICAL FIELD

The present disclosure relates to the use of a neurokinin-1 (NK-1) antagonist such as serlopitant, and optionally one or more additional antipruritic or therapeutic agents, in treating acute or chronic pruritus associated with a variety of medical conditions, or/and the medical conditions themselves.

### BACKGROUND OF THE DISCLOSURE

Pruritus (itch) is an unpleasant sensation that provokes a desire to scratch. Pruritus can have its origin directly in the skin or call develop in the central nervous system (CNS) via hematogenic or neurogenic mediators. Pruritus is a common symptom of a broad range of medical conditions, including dermatological and systemic disorders. Chronic pruritus can be intense, intractable and incapacitating, increase the disease severity, and greatly diminish the quality of life including causing sleep difficulty. Persistent rubbing or scratching can form secondary skin lesions such as excoriations, erosions, eschars, hyperpigmentation or patches of discoloration, impetiginisations and scars. Pruritus can induce an itch/scratch cycle and self-stimulation of the pruritic mechanism and scratching, which can exacerbate existing skin lesions and create new skin lesions. Chronic scratching worsens symptoms and often produces open skin lesions, which are susceptible to secondary infections, scarring and potential disfigurement. Once the itch/scratch cycle becomes established, it can be very difficult to stop.

Pruritus is a cutaneous sensory perception transmitted via unmyelinated C nerve fibers in the papillary dermis and epidermis of the skin, and is independent of pain. Itch receptors (pruriceptors) on cutaneous and spinal neurons process pruritic signals. Pruriceptors are present on the endings of unmyelinated C nerve fibers located in the papillary layer of the epidermis, with the highest number in the epidermis/dermis transition layer. Upon binding of histamine or other pruritogens to pruriceptors, histamine-sensitive or histamine-insensitive C fibers become depolarized and release pro-inflammatory neuropeptides such as substance P that evoke the pruritic signal or increase neuronal sensitivity to it. The release of such neuropeptides from afferent neurons cause neurogenic inflammation with symptoms including erythema, edema and burning itch. When pruriceptors are stimulated, the elicited neural impulse is transmitted to the dorsal root just outside the spinal cord. The cell bodies of afferent (including cutaneous) nerve fibers transmitting somatosensory information such as itch aggregate in the dorsal root ganglia. The impulse is transmitted further via the spinothalamic tract.

An important pruritus pathway is mediated by the neuropeptide substance P. Substance P is the most potent tachykinin and binds most strongly to neurokinin-1 (NK-1, also called tachykinin receptor 1 or substance P receptor) among the three tachykinin receptors NK-1, NK-2 and NK-3. NK-1 is expressed in the peripheral nervous system (PNS), including on keratinocytes and mast cells in the skin, and the CNS, including the dorsal root ganglia of spinal nerves and the brain. Substance P is an important mediator in both the PNS, including the skin, and the CNS during the induction and maintenance of pruritus. Substance P and NK-1 receptors are overexpressed in pruritic human skin. The skin of patients with atopic dermatitis and prurigo nodularis, both of which are characterized by severely itchy skin, and itchy burn scars following burn injury have a significantly greater density of substance P sensory nerve fibers compared with normal skin. Furthermore, injection of substance P into human skin causes symptoms of neurogenic inflammation such as erythema, edema and intense itch. Moreover, NK-1 receptors in the dorsal root ganglia of rats mediate scratching behavior. Substance P activates NK-1 in the PNS, including the skin, and in the CNS. The substance P/NK-1 interaction is important in mediating acute and chronic pruritus. Activation of NK-1 by substance P can generate an itch sensation in the PNS (e.g., dermal or neuropathic itch), including the skin, and the CNS (e.g., neurogenic or psychogenic itch).

The pruritogenic effect of substance P is intertwined with its pro-inflammatory effects. Upon depolarization, unmyelinated. C nerve fibers release substance P into the surrounding tissues. Substance P binds to NK-1 on keratinocytes and fibroblasts, thereby stimulating the secretion of histamine, interferon γ, interleukin-1β (IL-1β), IL-8 and nerve growth factor (NGF). Substance P binding to NK-1 on mast cells in the skin leads to degranulation and secretion of histamine, leukotriene B4, prostaglandin D2, IL-2, IL-8, tumor necrosis factor α, NGF, vascular endothelial growth factor (VEGF) and proteases (e.g., tryptase). The pro-inflammatory substances released from mast cells take part in the pathogenesis of pruritus. Furthermore, substance P binding to NK-1 on blood vessels leads to vasodilation and neurogenic inflammation, whose symptoms include erythema, edema and pruritus. Certain pruritogens including histamine, neuropeptides (e.g., substance P, gastrin-releasing peptide [GRP], neurotensin, somatostatin and vasoactive intestinal peptide [VIP]), interleukins (e.g., IL-31, whose receptor is expressed on cutaneous C nerve fibers and keratinocytes and in the dorsal root ganglia), and proteases (e.g., tryptase) provoke itch directly by binding to pruriceptors or indirectly by inducing release of histamine or other pruritogens. For example, histamine induces itch by stimulating the histamine H₁ and H₄ receptors on the endings of mechano-insensitive C nerve fibers in the skin (histamine also activates the histamine H₄ receptor on inflammatory cells including mast cells and T-lymphocytes [e.g., Th2 cells], thereby intensifying the pruritic signal), proteases (e.g., tryptase) activate the pruriceptor protease-activated receptor 2 (PAR2) on the endings of mechano-sensitive. C nerve fibers in the skin, and substance P and GRP induce itch by promoting release of various pruritogens such as histamine and proteases (e.g., tryptase) from, e.g., mast cells in the skin. Scratching provoked by itch damages the skin, consequently maintaining and reinforcing the inflammatory processes that induce further pruritus. Scratching results in local proliferation of skin nerves and increase in the levels of neuropeptides including substance P, which leads to increased secretion of cytokines and other pro-inflammatory mediators and stimulation of keratinocytes, fibroblasts and mast cells, thereby creating an itch/scratch cycle.

### SUMMARY OF THE DISCLOSURE

The present disclosure provides for the use of an antagonist (or inhibitor) of neurokinin-1 (NK-1) in treating acute or chronic pruritus associated with a variety of medical conditions, including dermatitis/eczema (e.g., atopic dermatitis), psoriasis (e.g., plaque psoriasis), prurigo (e.g., prurigo nodularis), urticaria (e.g., chronic idiopathic urticaria), cutaneous T-cell lymphoma (e.g., mycosis fungoides), epidermolysis bullosa (e.g., EB simplex), burns (e.g., thermal burns), and hepato-biliary diseases (e.g., cholestasis and primary biliary cirrhosis), or/and the medical conditions themselves. In some embodiments, the NK-1 antagonist is a selective NK-1 antagonist. In certain embodiments, the NK-1 antagonist is serlopitant, or a pharmaceutically acceptable salt, solvate, hydrate, clathrate, polymorph, prodrug or metabolite thereof.

In some embodiments, a therapeutically effective amount of the NK-1 antagonist (e.g., serlopitant) for the treatment of acute or chronic pruritus associated with a condition described herein is about 0.1-200 rug, 0.1-150 rag, 0.1-100 mg, 0.1-50 rag, 0.1-30 rag, 0.5-20 rug, 0.5-10 mg or 1-10 rag (e.g., per day or per dose), which can be administered in a single dose or in divided doses. In certain embodiments, the therapeutically effective dose (e.g., per day or per dose) of the NK-1 antagonist (e.g., serlopitant) for treating acute or chronic pruritus associated with a condition described herein is about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 rag, 5 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 rag (e.g., about 100 mg, 125 mg or 150 mg), or about 150-200 rag (e.g., about 150 mg, 175 mg or 200 mg). In some embodiments, the therapeutically effective dose of the NK-1 antagonist (e.g., serlopitant) is administered one or more (e.g., two) times a day, or once every two or three days, or once, twice or thrice a week. In certain embodiments, the therapeutically effective dose of the NK-1 antagonist (e.g., serlopitant) is administered once daily. In further embodiments, the therapeutically effective dose of the NK-1 antagonist (e.g., serlopitant) is about 0.5-5 mg, 1-5 rag or 5-10 mg (e.g., about 0.5 mg, 1 mg, 5 rag or 10 rug) once daily. In certain embodiments, the therapeutically effective dose of the NK-1 antagonist (e.g., serlopitant) is about 5 mg once daily.

The NK-1 antagonist (e.g., serlopitant) can also be dosed in irregular manner. For example, the NK-1 antagonist can be administered once, twice or thrice in a period of two weeks, three weeks or a month in an irregular manner. Furthermore, the NK-1 antagonist (e.g., serlopitant) can be taken pro re nata (as needed). For instance, the NK-1 antagonist can be administered 1, 2, 3, 4, 5 or more times, whether in a regular or irregular manner, until pruritus improves. Once relief from itch is achieved, dosing of the NK-1 antagonist can optionally be discontinued. If pruritus returns, administration of the NK-1 antagonist, whether in a regular or irregular manner, can be resumed. The appropriate dosage of, frequency of dosing of and length of treatment with the NK-1 antagonist can be determined by the treating physician.

In some embodiments, the NK-1 antagonist (e.g., serlopitant) is administered under a chronic dosing regimen for the treatment of chronic pruritus associated with a condition described herein. In certain embodiments, a therapeutically effective amount of the NK-1 antagonist (e.g., serlopitant) is administered over a period of at least about 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months). Administration of the NK-1 antagonist (e.g., serlopitant) over a period of less than about 6 weeks (e.g., for about 1 week, 2 weeks, 3 weeks, 4 weeks or 5 weeks) can be regarded as treatment of acute pruritus.

In certain embodiments, the NK-1 antagonist (e.g., serlopitant) is administered at bedtime (e.g., once daily at bedtime). The NK-1 antagonist can also be administered at any appropriate time daring the day or awake hours (e.g., in the morning). In further embodiments, the NK-1 antagonist (e.g., serlopitant) is administered without food (e.g., at least about 1 or 2 hours before or after a meal, such as at least about 2 hours after an evening meal). The NK-1 antagonist can also be taken substantially concurrently with food (e.g., within about 0.5, 1 or 2 hours before or after a meal, or with a meal).

In certain embodiments, the NK-1 antagonist (e.g., serlopitant) is administered orally (e.g., as a capsule or tablet, optionally with an enteric coating). In other embodiments, the NK-1 antagonist (e.g., serlopitant) is administered parenterally (e.g., intravenously, subcutaneously or intradermally). In further embodiments, the NK-1 antagonist (e.g., serlopitant) is administered topically (e.g., dermally/epicutaneously, transdermally, mucosally, transmucosally, buccally or sublingually).

For the treatment of chronic pruritus associated with a condition described herein, in certain embodiments the NK-1 antagonist (e.g., serlopitant) is administered in a dose of about 0.5, 1, 5 or 10 mg (e.g., about 5 mg) orally (e.g., as a tablet) once daily for at least about 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).

In some embodiments, the NK-1 antagonist (e.g., serlopitant) is administered to a subject for the treatment of acute or chronic pruritus associated with a condition described herein according to a dosing schedule, wherein at least one loading dose is first administered (e.g., to establish more quickly a therapeutically effective dose in the subject), and at least one therapeutically effective maintenance dose is subsequently administered. The therapeutically effective maintenance dose can be any therapeutically effective dose described herein. In some embodiments, the loading dose is about five times, four times, three times or two times greater than the maintenance dose. In certain embodiments, the loading dose is about three times greater than the maintenance dose. In some embodiments, the loading dose is administered on day 1 and the maintenance dose is administered on day 2 and thereafter. In some embodiments, the NK-1 antagonist (e.g., serlopitant) is administered in a loading dose of about 1.5, 3, 15 or 30 mg (e.g., 3×about 0.5, 1, 5 or 10 mg) orally (e.g., as a tablet) on day 1, followed by a maintenance dose of about 0.5, 1, 5 or 10 mg orally (e.g., as a tablet) once daily, optionally at bedtime, for at least about 2 weeks, 1 month (4 weeks), 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months). In certain embodiments, the NK-1 antagonist (e.g., serlopitant) is administered in a loading dose of about 15 mg (e.g., 3×about 5 mg) orally (e.g., as a tablet) on day 1, followed by a maintenance dose of about 5 mg orally (e.g., as a tablet) once daily, optionally at bedtime, for at least about 2 weeks, 1 month, 6 weeks, 2 months, 3 months, 6 months, 1 year, 1.5 years, 2 years, 3 years or longer (e.g., at least about 6 weeks, 2 months, 3 months or 6 months). In further embodiments, a second loading dose is administered prior to administering the maintenance dose. In certain embodiment, the first loading dose is about three times greater than the maintenance dose, and the second loading dose is about two times greater than the maintenance dose.

In some embodiments, one or more additional antipruritic or therapeutic agents in addition to an NK-1 antagonist (e.g., serlopitant) are administered for the treatment of acute or chronic pruritus associated with a medical condition described, herein, or/and the medical condition itself.

An NK-1 antagonist (e.g., serlopitant) can be used to treat other symptoms or complications of a medical condition described herein besides pruritus, or can be used to treat the medical condition itself. For example, the NK-1 antagonist (e.g., serlopitant) can be administered to slow the progression or to reduce the severity of the medical condition, to improve the health or/and the function of a tissue or organ (e.g., skin or liver), to decrease the number, frequency, area, extent or/and severity of skin symptoms (e.g., skin lesions, rashes, flares, nodules, papules, plaques, blisters and wheals), or to improve wound healing (e.g., reduce wound surface area and reduce the number and size of open sores), or any combination thereof, wherein the additional therapeutic benefits may or may not result from reduction in itch and scratching (e.g., they may be due to the NK-1 antagonist's anti-inflammatory, anti-proliferative or/and anti-metastatic effects).

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1 illustrates a Franz diffusion cell for studying skin permeation of a drug vitro.

FIG. 2 shows the cumulative release of serlopitant from topical formulations B and C into the receptor chamber of a Franz diffusion cell at various time points in an in vitro study of skin permeation.

FIG. 3 shows the amount of serlopitant (called “VPD737”) retained in the skin at the end of the Franz diffusion cell study. Each bar represents ug of serlopitant/g of skin in 250 urn skin layers. For each of topical formulations B and C, the bars from left to right represent the amount of serlopitant retained in skin layers from the stratum contemn to the dermis.

### DETAILED DESCRIPTION OF THE DISCLOSURE

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein may be employed in practicing the disclosure. It is also understood that every embodiment of the disclosure may optionally be combined with any one or more of the other embodiments described herein which are consistent with that embodiment.

Where elements are presented in list format in a Markush group), it is understood that each possible subgroup of the elements is also disclosed, and any one or more elements can be removed from the list or group.

It is also understood that, unless clearly indicated to the contrary, in any method described or claimed herein that includes more than one act, the order of the acts of the method is not necessarily limited to the order in which the acts of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, any embodiment found within the prior art, can be explicitly excluded from the claims, regardless of whether or not the specific exclusion is recited in the specification.

It is further understood that the present disclosure encompasses analogs, derivatives, prodrugs, metabolites, salts, solvates, hydrates, clathrates and polymorphs of all of the compounds/substances disclosed herein, as appropriate. The specific recitation of “analogs”, “derivatives”, “prodrugs”, “metabolites”, “salts”, “solvates”, “hydrates”, “clathrates” or “polymorphs” with respect to a compound/substance or a group of compounds/substances in certain instances of the disclosure shall not be interpreted as an intended omission of any of these forms in other instances of the disclosure where the compound/substance or the group of compounds/substances is mentioned without recitation of any of these forms.

Headings au included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### Definitions

Unless defined otherwise or indicated otherwise by their use herein, all technical and scientific terms used herein have the same meaning as commonly understood by one of ordinary skill in the art to which this application belongs.

As used in the specification and the appended claims, the indefinite articles “a” and “an” and the definite article “the” can include plural referents as well as singular referents unless specifically stated otherwise or the context clearly dictates otherwise.

The abbreviation “aka” denotes “also known as”.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 20%, 15%, 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” “less than” applies to each one of the numerical values in that series of numerical values.

The term “antagonists” includes neutral antagonists and inverse agonists.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” carrier or excipient of a pharmaceutical composition is also compatible with the other ingredients of the composition.

The term “therapeutically effective amount” refers to an amount of a substance that, when administered to a subject, is sufficient to prevent, reduce the risk of developing, delay the onset of, or slow the progression of the medical condition being treated, or to alleviate to some extent one or more symptoms or complications of that condition. The term “therapeutically effective amount” also refers to an amount of a substance that is sufficient to elicit the biological or medical response of a cell, tissue, organ, system, animal or human which is sought by a researcher, veterinarian, medical doctor or clinician.

The terms “treat”, “treating” and “treatment” include alleviating or abrogating a medical condition or one or more symptoms or complications associated with the condition, and alleviating or eradicating one or more causes of the condition. Reference to “treatment” of a medical condition includes preventing (precluding), reducing the risk of developing, delaying the onset of, and slowing the progression of, the condition or one or more symptoms or complications associated with the condition.

The term “medical conditions” (or “conditions” for short) encompasses disorders and diseases.

The term “subject” refers to an animal, including a mammal, such as a primate (e.g., a human, a chimpanzee or a monkey), a rodent (e.g., a rat, a mouse, a guinea pig, a gerbil or a hamster), a lagomorph (e.g., a rabbit), a swine (e.g., a pig), an equine (e.g., a horse), a canine (e.g., a dog) or a feline (e.g., a cat). The terms “subject” and “patient” are used interchangeably herein in reference, e.g., to a mammalian subject, such as a human subject.

### Pruritus Associated Conditions

Dermatitis, also known as eczema, is a group of skin conditions characterized by inflammation of the skin. Common symptoms of these skin conditions include itchiness, redness of the skin (erythema), skin lesions, rashes and skin swelling. The primary symptom is itchy skin. The area of skin affected can vary from small to the entire body. Types of dermatitis/eczema include without limitation atopic dermatitis, papular dermatitis (aka itchy red bump disease), xerotic eczema (aka asteatotic eczema, eczema craquele or desiccation dermatitis), exfoliative dermatitis (aka erythroderma), discoid eczema (aka nummular eczema), hand or/and foot eczema (e.g., hyperkeratotic hand or/and foot eczema, vesicular palmoplantar dermatitis [aka dyshidrosis, dyshidrotic eczema or pompholyx] and chronic vesiculobullous hand eczema), intertrigo dermatitis, perioral dermatitis, contact dermatitis (e.g., allergic contact dermatitis, irritant contact dermatitis, aquagenic dermatitis and phototoxic dermatitis), seborrheic dermatitis (e.g., infantile seborrheic dermatitis. Leiner's disease and pityriasis simplex capillitii [dandruff]), pustular dermatitis (e.g., eosinophilic pustular folliculitis [aka Ofuji's disease]), stasis dermatitis (aka gravitational eczema, varicose eczema or venous eczema), autosensitization dermatitis (aka autoeczematization, generalized eczema or id reaction, whether or not related to an infection), infection-related dermatitis (e.g., Kaposi varicelliform eruption [aka Kaposi-Juliusberg dermatitis, pustulosis varioliformis acute or eczema herpeticum], cercarial dermatitis [aka swimmer's itch], dermatitis gangrenosa and eczema vaccinatum), dermatitis resulting from an underlying disease (e.g., celiac disease [such as dermatitis herpetiformis {aka Duhring's disease}] or lymphoma), dermatitis resulting from ingestion of a substance (e.g., food, a medication or a chemical), neurodermatitis (aka lichen simplex chronicus), chronic superficial dermatitis (aka small plaque parapsoriasis), and lichenoid dermatitis (e.g., cutaneous lichen planus).

Atopic dermatitis (AD) is the most common type of dermatitis, affects about 10-20% of people, is typically chronic, and is frequently referred to as “the itch that rashes”. AD is characterized by dry, itchy, red, swollen and cracked skin. People with AD often have dry and scaly skin over the entire body, and intensely itchy, red, splotchy, raised lesions forming in the bends of the arms or the legs, the face, and the neck. Pruritus is present in nearly all AD subjects. AD typically begins in childhood with changing severity over the years. As children become older, the back of the knees and the front of the elbows are the most common areas for the rash. In adults, the hands and the feet are the most affected areas.

Psoriasis is a typically chronic, immune-mediated inflammatory and proliferative skin disease characterized by patches that are typically itchy, red and scaly. The skin patches can vary from small and localized to complete body coverage. Pruritus is reported by patients as the most bothersome symptom of psoriasis. Injury to the skin can induce psoriatic skin lesions at that spot, which is known as the Koebner phenomenon. Psoriasis affects about 2-4% of people. Types of psoriasis include without limitation plaque psoriasis (aka psoriasis vulgaris or chronic stationary psoriasis), guttate psoriasis (aka eruptive psoriasis), inverse psoriasis (aka flexural psoriasis), pustular psoriasis, seborrheic-like psoriasis and erythrodermic psoriasis. Plaque psoriasis constitutes about 85-90% of psoriasis cases and typically appears as raised areas of inflamed skin covered with silvery-white scaly skin. Such areas are called plaques and are most commonly found on the back of the forearms, elbows, shins, knees, the area around the navel, the back, and the scalp. Erythrodermic psoriasis involves widespread inflammation and exfoliation of the skin over most of the body surface, and can be accompanied by severe itching, swelling and pain. It can develop from any of the other types of psoriasis, but often results from an exacerbation of unstable plaque psoriasis, particularly following the abrupt withdrawal of a systemic glucocorticoid.

Prurigo is an itchy eruption of the skin. Prurigo is manifested by the formation of usually small exudative nodules that are swollen and red at the base, sometimes with a minor blister filled with serous fluid. Types of prurigo include without limitation prurigo nodularis, prurigo simplex, actinic prurigo, Besnier prurigo (aka prurigo gestationis, prurigo of pregnancy and papular dermatitis of pregnancy), prurigo dermographica, prurigo pigmentosa, and psychogenic prurigo (e.g., somatoform prurigo and depression-associated prurigo). Prurigo simplex is a typically chronic and idiopathic skin condition characterized by intensely itchy skin nodules and lesions that appear most commonly on die scalp, the arms, the legs, and the trunk of the body. Prurigo simplex also occurs in acute and subacute forms. Actinic prurigo is a common, typically chronic, sunlight-induced skin eruption characterized by itchy, inflamed skin papules, nodules and plaques that appear most frequently on the face, the neck, the arms, the hands, and the legs.

Prurigo nodularis (PN) is a typically chronic skin condition characterized by severely itchy papulonodular skin eruptions that typically appear on the arms or/and the legs, although such eruptions can also be present on the trunk of the body. PN is associated with a diverse range of diseases. PN subjects usually have chronic severe pruritus and very itchy skin nodules and excoriated lesions caused by chronic scratching. PN is also known as Hyde prurigo nodularis. Picker's nodules, atypical nodular form of neurodermatitis circumscripta, and lichen cornet's obtusus.

Urticaria, also known as hives, is a kind of skin rash characterized by pale red, raised bumps that itch and may also burn or sting. The skin lesions of urticaria are caused by an inflammatory reaction in the skin that is triggered by the release of pro-inflammatory substances (e.g., histamine, cytokines and leukotrienes) from cells (e.g., mast cells) in the skin. The inflammatory reaction causes leakage of capillaries in the dermis, and results in an edema that persists until the interstitial fluid is absorbed into the surrounding cells. Pruritus is present in nearly all urticaria subjects. Severe pruritus often occurs, and pruritus is most severe in the phase of wheal formation. Most cases of hives lasting less than six weeks (acute urticaria) are caused by an allergic reaction. Chronic urticaria (hives lasting six weeks or longer) usually is not clue to an allergy. The majority of chronic urticaria cases last a year or more, and about 20% of the cases last 20 years or more. Acute urticaria occurs in about 20-30% of people, while chronic urticaria affects about 2-5% of people.

Acute urticaria is characterized by wheals that completely resolve within six weeks. Acute urticaria is often caused by an allergic reaction to food (e.g., eggs, nuts, shellfish, soy, wheat and food additives [e.g., Balsam of Peru]), insect (e.g., bee and wasp) stings, and fragrances. Acute viral infection (e.g., that causing the common cold) is another common cause of acute urticaria (viral exanthem). Less common causes of acute urticaria include friction, pressure (e.g., tight clothing), water, sunlight, extreme heat and cold, exercise, emotional stress, fever, and drugs (e.g., dextroamphetamine, piracetam, antibiotics [e.g., cefaclor, metronidazole and penicillin], antifungal drugs [e.g., clotrimazole], anticonvulsants, antidiabetics [e.g., glimepiride], non-steroidal anti-inflammatory drugs [NSAIDs, e.g., aspirin and ibuprofen], opioids [e.g., codeine and morphine] and sulfonamides).

Chronic urticaria is characterized by wheals that persist for six weeks or longer. A common type of chronic urticaria is cold urticaria, which is caused by exposure to extreme cold and lasts 5-6 years on average. Chronic urticaria can also be a complication or symptom of a parasitic infection (e.g., blastocystosis and strongyloidiasis). Furthermore, chronic urticaria can be induced by drugs (e.g., NSAIDs such as aspirin and ibuprofen).

The majority of chronic urticaria cases have an unknown cause (chronic idiopathic urticaria [CIU]). Perhaps more than 50% of CIU cases are caused by an autoimmune reaction: roughly 50% of subjects with chronic urticaria spontaneously develop autoantibodies directed at the receptor FcεRI on mast cells in the skin, and chronic stimulation of this receptor results in chronic urticaria. CIU is also linked to emotional stress (e.g., bereavement, divorce and post-traumatic stress). One of the most common types of chronic urticaria is dermatographic urticaria (aka dermatographism), which is marked by the appearance of itchy wheals or welts on the skin as a result of scratching or firm stroking of the skin and occurs in about 2-5% of the population. The cause of most cases of dermatographism is unknown, although it may be preceded by, e.g., a viral infection, antibiotic therapy or emotional upset.

Cutaneous T-cell lymphoma (CTCL) is a class of non-Hodgkin lymphoma caused by a mutation in T cells. The malignant T cells in the body initially migrate to the skin and cause lesions there. The lesions typically begin as very itchy rashes and eventually form plaques and tumors before metastasizing to other parts of the body. The symptoms of CTCL can be debilitating and painful, even in the earlier stages of CTCL. Pruritus is a very common symptom of CTCL, develops at an early stage of CTCL, and becomes more intense as the disease progresses. Types of CTCL include without limitation mycosis fungoides (MF) and forms and variants thereof (e.g., erythrodermic MF, granulomatous slack skin, pagetoid reticulosis and Sézary syndrome), CD30+ CTCL, secondary cutaneous CD30+ large cell lymphoma (which may arise in cases of, e.g., MF and lymphomatoid papulosis), non-MF CD30 large-sized CTCL, pleomorphic T-cell lymphoma (aka non-MF CD30 pleomorphic small-/medium-sized CTCL), angiocentric lymphoma (aka extranodal NK-/T-cell lymphoma, nasal type), blastic NK-cell lymphoma, Lennert lymphoma, lymphomatoid, papulosis, pityriasis lichenoides chronica, pityriasis lichenoides et varioliformis acuta, and subcutaneous T-cell lymphoma.

Mycosis fungoides (aka granuloma fungoides) is the most common type of CTCL. It generally affects the skin, but may progress internally over time. Symptoms of MF include itchy skin, rashes, skin lesions and tumors. MF consists of three stages. The premycotic stage presents as itchy, erythematous (red), scaly lesions and often resembles eczema or psoriasis. In the mycotic stage, infiltrative plaques appear as well as a polymorphous inflammatory infiltrate in the dermis. In the tumorous stage, a dense infiltrate of medium-sized lymphocytes with cerebroid nuclei expands the dermis.

Epidermolysis bullosa (EB) is a group of inherited connective tissue diseases that form blisters in the skin and mucosal membranes. Over 300 mutations have been identified in EB. EB is a consequence of the epidermis and dermis lacking protein anchors that hold the skin layers together, which results in extremely fragile skin—even minor friction (e.g., rubbing) or minor trauma separates the layers of the skin and forms severe blisters and painful sores. EB patients liken the sores to third-degree burns. As a complication of the chronic skin damage, EB patients have an increased risk of skin cancers. Pruritus is reported by patients of all EB types as the most bothersome EB symptom and as one of the most debilitating aspects of EB, ranking higher than acute or chronic pain or problem with eating. Types of EB include without limitation epidermolysis bullosa simplex (EBS), epidermolysis bullosa acquisita (EBA), dystrophic epidermolysis bullosa (DEB), junctional epidermolysis bullosa (JEB) and hemidesmosomal epidermolysis bullosa (HEB). A subtype of dystrophic EB is epidermolysis bullosa pruriginosa (EBP), which is characterized by marked itching and the presence of prurigo-like or lichenoid features, including itchy lichenoid lesions. About 90% of EB cases are EB simplex.

A burn is a type of injury to the skin or other tissues that can be caused by any source. A burn injury is generally caused by heat (e.g., fire/flame, hot liquids and gases, and hot objects), electricity, chemicals (e.g., strong bases and strong acids), friction, or radiation (e.g., ultraviolet light [such as sunburn] and ionizing radiation [such as from radiation therapy, X-ray or radioactive fallout]). Most burns are caused by heat from fire or hot liquids. In the United States, the most commonly reported causes of burns are fire or flame (44%), scalds (33%), hot objects (9%), electricity (4%), and chemicals (3%). In the United States alone, over 450,000 cases of burn injuries requiring medical treatment are reported annually. Pruritus is a prevalent and persistent symptom following burn injury, is common during, the healing process (occurring in about 90% of adults and nearly all children), and is a significant cause of morbidity in burn survivors. Risk factors for pruritus severity include burn depth, extent of burned total body surface area (TBSA), and extent of skirt-grafted TBSA.

Burns can be classified by, e.g., mechanism of injury, depth of injury, and severity of injury. Burns classified by mechanism of injury include without limitation thermal burns, electrical burns, chemical burns, friction burns and radiation burns. Burns classified by depth of injury include without limitation first-degree burns, second-degree burns, third-degree burns and fourth-degree burns. Classification of burns by severity is based on factors such as percentage of TBSA affected, burn depth, affected anatomical zones, the age of the person, and associated injuries. Burns classified by severity of injury include without limitation minor burns, moderate burns and major burns. In large burns (over about 30% of the IBSA), there is a significant inflammatory response, which results in increased leakage of fluid from capillaries and subsequent tissue edema.

Hepato-biliary diseases can result from a wide variety of causes. For example, fatty liver disease can be caused by excessive alcohol consumption or obesity and hepatitis can be caused by excessive alcohol consumption or a viral infection (e.g., hepatitis B or C), and both diseases can lead to cirrhosis and liver failure. Furthermore, a broad range of commonly used drugs such as antibiotics, NSAIDs and statins can cause hepato-biliary diseases such as cholestasis. Cholestasis can also be due to a wide variety of hepato-biliary diseases. Pruritus is a common symptom of many hepato-biliary diseases, including but not limited to cholestatic disorders, hepatitis (e.g., chronic hepatitis B and C and autoimmune hepatitis), cirrhosis and liver failure (other than complete liver failure).

The term “hepato-biliary diseases” includes liver diseases, gallbladder diseases and biliary tract diseases. Non-limiting examples of liver diseases (including intrahepatic diseases and extrahepatic diseases) include:
alcoholic liver diseases, including but not limited to fatty liver disease, hepatitis, steatohepatitis, fibrosis, sclerosis, cirrhosis and liver failure;
liver diseases caused by drugs and toxins other than alcohol, including but not limited to cholestasis, cirrhosis, fibrosis, granuloma, hepatitis (including acute and chronic hepatitis), liver failure (including acute and chronic liver failure), necrosis, steatosis, vascular disorders (e.g., hepatic vein thrombosis, peliosis hepatis and veno-occlusive disease), and benign and malignant neoplasms (e.g., hepatic adenomas, hepatic angiosarcoma and hepatocellular carcinoma);
viral hepatitis (including acute and chronic viral hepatitis), including but not limited to hepatitis A, hepatitis B, hepatitis C, hepatitis D, hepatitis E, and hepatitis caused by other viruses (e.g., cytomegalovirus, Epstein-Barr virus, herpes simplex virus, rubella virus and yellow fever virus);
liver diseases caused by other infectious or parasitic agents, including but not limited to amoebas (e.g., amoebiasis), bacteria (e.g., leptospirosis and syphilis), parasitic warms (e.g., echinococcosis, fasciolosis and schistosomiasis), and protozoa (e.g., toxoplasmosis);
inflammatory liver diseases other than viral hepatitis, including but not limited to autoimmune hepatitis, granulomatous hepatitis (e.g., berylliosis and sarcoidosis), liver abscess, non-alcoholic steatohepatitis (NASH), phlebitis of the portal vein, primary biliary cholangitis (aka primary biliary cirrhosis), and primary sclerosing cholangitis;
metabolic liver diseases, including but not limited to cholestasis, hemochromatosis, non-alcoholic fatty liver disease (e.g., NASH), glycogen storage disease type II (Pompe disease), glycogen storage disease type IV, Crigler-Najjar syndrome, Dubin-Johnson syndrome, Gilbert's syndrome, Rotor's syndrome and Wilson's disease;
vascular liver disorders, including but not limited to Budd-Chiari syndrome, central hemorrhagic necrosis of the liver, chronic passive congestion of the liver, infarction of the liver, peliosis hepatis, portal hypertension and veno-occlusive disease;
chronic and end-stage liver diseases, including but not limited to hepatitis, hepatopulmonary syndrome, hepatorenal syndrome, hepatotoxicity, portal hypertension, fibrosis, cirrhosis and liver failure;
benign and malignant neoplasms, tumors and cancers of the liver and intrahepatic bile ducts, including but not limited to focal nodular hyperplasia, hepatic adenomas, hepatic hemangiomas, hepatoblastoma, carcinomas (e.g., cholangiocarcinoma and hepatocellular carcinoma [malignant hepatoma]), and sarcomas (e.g., angiosarcoma of the liver, hemangiosarcoma of the liver and Kupffer cell sarcoma);
cyst-related liver diseases, including but not limited to congenital cystic disease of the liver, polycystic liver disease and cysts caused by Echinococcus; and
other liver diseases, including but not limited to amyloid degeneration of the liver (e.g., transthyretin-related hereditary amyloidosis).

Examples of gallbladder diseases include without limitation cholecystitis, cholelithiasis, cholestasis, cholesterolosis, edema, fistula, obstruction, perforation, and benign and malignant neoplasms, tumors and cancers of the gallbladder. Examples of biliary tract diseases include without limitation biliary atresia, biliary cyst, biliary dyskinesia, cholangitis (including ascending cholangitis and primary sclerosing cholangitis), cholestasis, choledocholithiasis, fistula, obstruction, perforation, and benign and malignant neoplasms, tumors and cancers of the biliary tract (including cancers of the extrahepatic bile ducts and the ampulla of Vater).

A type of hepato-biliary disease may be included in more than one category, or in all categories, among liver diseases, gallbladder diseases and biliary tract diseases. For example, cholestasis can be caused by a disorder of the liver (which produces bile), the gallbladder (which stores bile) or the biliary tract (the conduit for bile to flow from the liver and the gallbladder to the small intestine). Therefore, cholestasis can be categorized as a liver disease, a gallbladder disease and a biliary tract disease.

Cholestasis is impairment (slowing or stopping) of bile flow, which results in hyperbilirubinemia. Cholestasis can be caused by diseases of the liver, the gallbladder or the biliary tract. Causes of cholestasis include without limitation biliary atresia, biliary trauma, hereditary or congenital anomalies/defects of the biliary tract (e.g., progressive familial intrahepatic cholestasis [Byler's disease] caused by defects in biliary epithelial transporters), primary biliary cirrhosis, primary sclerosing cholangitis, gallstones in the biliary tract, the gallbladder and the liver, acute and chronic hepatitis, abdominal mass (e.g. cancer), pregnancy (e.g., intrahepatic cholestasis of pregnancy), cystic fibrosis and drugs (info). The two basic types of cholestasis are obstructive (e.g., a blockage of the duct system due to, e.g., a gallstone or a malignancy) and metabolic (e.g., a disturbance in bile formation due to a genetic defect or a drug). Pruritus is the primary symptom of cholestasis, and may be caused by bile acids/bile, salts in the skin or/and the bloodstream, possibly as a result of their activation of the mu-opioid receptor expressed by nerves, or caused by substances secreted with bile and reabsorbed from the intestines into the bloodstream. Lysophosphatidic acid (LPA) may also cause cholestatic pruritus. While cholestatic pruritus can be due to nearly any hepato-biliary disease in addition to cholestasis, it is more commonly associated with, e.g., obstructive choledocholithiasis, primary biliary cirrhosis, primary sclerosing cholangitis, carcinoma of the bile duct, and viral hepatitis (e.g., chronic hepatitis C).

Primary biliary cirrhosis (PBC), also known as primary biliary cholangitis, is an autoimmune, inflammatory disease of the liver characterized by progressive destruction of the bile ducts (e.g., the interlobular bile ducts) of the liver. When the ducts are damaged, bile and other toxins build up in the liver (cholestasis), which damages the liver tissue along with ongoing, immune-related damage. PBC can lead to scarring, fibrosis and cirrhosis. PBC can be regarded as a type of obstructive cholestasis. Pruritus is a common symptom of PBC. Other cholestatic disorders characterized by pruritus include without limitation primary sclerosing cholangitis and cystic fibrosis.

Cirrhosis is a condition in which the liver does not function properly due to long-term damage. Cirrhosis is characterized by replacement of normal liver tissue by scar tissue, which leads to loss of liver function. Pruritus is a common symptom of cirrhosis as the disease worsens. Cirrhosis is most commonly caused by, e.g., chronic hepatitis B, chronic hepatitis C, alcoholic liver disease, and non-alcoholic fatty liver disease (e.g., NASH). Other causes of cirrhosis include, e.g., autoimmune hepatitis, PBC, primary sclerosing cholangitis, gallstones, hemochromatosis, Wilson's disease, alpha 1-antitrypsin deficiency (A1AD), Indian childhood cirrhosis, cardiac cirrhosis, galactosemia, glycogen storage disease type IV, cystic fibrosis, and hepatotoxic drugs and toxins.

Pruritus is also a common symptom of liver failure (other than complete liver failure because the liver is unable to produce pruritogenic substances in such a state). Liver failure (aka hepatic insufficiency) is the inability of the liver to perform its normal synthetic and metabolic functions. The two basic forms of liver failure are acute and chronic liver failure. Chronic liver failure usually occurs in the context of cirrhosis.

Pruritus can also be induced by a drug or toxin that causes a hepato-biliary disease or liver damage/injury (hepatotoxicity). Non-limiting examples of drug-induced liver diseases include cholestasis (e.g., with allopurinol, carbamazepine, chlorpromazine, prochlorperazine, sulindac, antibiotics [e.g., amoxicillin/clavulanic acid {co-amoxiclav}, erythromycin, flucloxacillin nitrofurantoin, and trimethoprim/sulfamethoxazole {TMP/SMX or co-trimoxazole}], statins, anabolic steroids, estrogen, androgens, oral contraceptive pills and gold salts), granuloma (e.g., with allopurinol, isoniazid, penicillin, phenytoin, quinine and quinidine), acute and chronic hepatitis (e.g., with aspirin, diclofenac, halothane isoniazid, methyldopa and phenytoin), acute and chronic liver failure (e.g., with acetaminophen), necrosis (e.g., with acetaminophen), steatosis (e.g., with acetaminophen, amiodarone, aspirin, ketoprofen, methotrexate and tetracycline), vascular disorders (e.g., hepatic vein thrombosis [e.g., with oral contraceptives], peliosis hepatis [e.g., with anabolic steroids] and veno-occlusive disease [e.g., with chemotherapeutic drugs]), and benign and malignant neoplasms and tumors (e.g., hepatic adenomas, hepatic angiosarcoma and hepatocellular carcinoma [e.g., with anabolic steroids, the combined oral contraceptive pill and thorotrast, and with industrial toxins such as arsenic and vinyl chloride). Examples of other hepatotoxins include without limitation ketoconazole, hydrazine-containing drugs (e.g., iproniazid, isoniazid and phenelzine), NSAIDs (e.g., aspirin, diclofenac, ibuprofen, indomethacin, phenylbutazone, piroxicam and sulindac), and industrial toxins arsenic, carbon tetrachloride and vinyl chloride).

### Treatment of Pruritus Using a Neurokinin-1 Antagonist

Pruritus (itch) is a common symptom of the medical conditions described herein, and can be severe, intractable and incapacitating. Itch often triggers scratching that creates new skin lesions, exacerbates existing skin lesions, and worsens the condition. Treatment of pruritus with standard antipruritic therapies such as oral H₁ antihistamines, topical corticosteroids and emollients does not provide significant relief of itch in many or most patients.

The interaction between substance P and neurokinin-1 (NK-1) is a key transmitter of the itch signal. Substance P is the most potent tachykinin and binds most strongly to NK-1 among the three tachykinin receptors NK-1, NK-2 and NK-3. By inhibiting NK-1 or blocking binding of substance P to NK-1, an NK-1 antagonist blocks the transmission of itch from the skin to the CNS. Use of an NK-1 antagonist can reduce the incidence and intensity of pruritus associated with a condition described herein, minimize damage to the skin, decrease disease severity and significantly increase the quality of life of patients.

The present disclosure provides for the use of an NK-1 antagonist in treating acute or chronic pruritus associated with a condition described herein. In some embodiments, the acute or chronic pruritus is associated with dermatitis or eczema. The acute or chronic pruritus can be associated with any and all types of dermatitis or eczema. In some embodiments, the dermatitis or eczema is atopic dermatitis, papular dermatitis, xerotic eczema, contact dermatitis (e.g., allergic contact dermatitis or irritant contact dermatitis), seborrheic dermatitis, pustular dermatitis, stasis dermatitis, neurodermatitis (aka lichen simplex chronicus) or lichenoid dermatitis (e.g., cutaneous lichen planus). In certain embodiments, the dermatitis or eczema is atopic dermatitis.

In further embodiments, the acute or chronic pruritus is associated with psoriasis. The acute or chronic pruritus can be associated with any and all types of psoriasis. In certain embodiments, the psoriasis is plaque psoriasis (aka psoriasis vulgaris).

In still further embodiments, the acute or chronic pruritus is associated with prurigo. The acute or chronic pruritus can be associated with any and all types of prurigo. In some embodiments, the prurigo is prurigo nodularis, prurigo simplex or actinic prurigo. In certain embodiments, the prurigo is prurigo nodularis.

In yet further embodiments, the acute or chronic pruritus is associated with urticaria. The acute or chronic pruritus can be associated with any and all types of urticaria, including acute and chronic urticaria and including urticaria cases having known or unknown (idiopathic) causes. In certain embodiments, the urticaria is chronic idiopathic urticaria.

In additional embodiments, the acute or chronic pruritus is associated with cutaneous T-cell lymphoma (CTCL). The acute or chronic pruritus can be associated with any and all types of CTCL. In certain embodiments, the CTCL is mycosis fungoides or a form or variant thereof (e.g., erythrodermic mycosis fungoides, granulomatous slack skin, pagetoid reticulosis or Sezary syndrome).

In other embodiments, the acute or chronic pruritus is associated with epidermolysis bullosa (EB). The acute or chronic pruritus can be associated with any and all types of EB. In certain embodiments, the EB is epidermolysis bullosa simplex.

In still other embodiments, the acute or chronic pruritus is associated with a burn, including post-burn pruritus. As used herein, the term “post-burn pruritus” includes acute and chronic pruritus following burn injury, which also encompasses acute and chronic pruritus associated with or resulting from healing/repair of the burn injury or wound, including scar formation. The acute or chronic pruritus can be associated with any and all types of burns, which can be classified by, e.g., mechanism of injury (e.g., thermal, electrical, chemical, friction and radiation), depth of injury (e.g., first degree, second degree, third degree and fourth degree), and severity of injury (e.g., minor, moderate and major). In certain embodiments, the pruritus is associated with a thermal burn. In further embodiments, the pruritus is associated with a second-degree burn or a third-degree burn. In other embodiments, the pruritus is associated with a moderate burn or a major burn.

In additional embodiments, the acute or chronic pruritus is associated with a hepato-biliary disease. The acute or chronic pruritus can be associated with any and all types of liver diseases, gallbladder diseases and biliary tract diseases. Furthermore, the acute or chronic pruritus can be induced by drugs or toxins that cause a hepato-biliary disease or liver damage/injury. In some embodiments, the hepato-biliary disease is selected from cholestatic disorders; cholestasis; progressive familial intrahepatic cholestasis; intrahepatic cholestasis of pregnancy; biliary atresia; primary binary cirrhosis (primary biliary cholangitis); primary sclerosing cholangitis; obstructions of the biliary tract, the gallbladder and the liver; gallstones in the biliary tract, the gallbladder and the liver; choledocholithiasis; cholelithiasis; biliary cysts; benign and malignant neoplasms and tumors (including carcinomas) of the binary tract, the gallbladder and the liver; cystic fibrosis; biliary dyskinesia; alcoholic and non-alcoholic fatty liver disease; acute and chronic hepatitis (including viral hepatitis [including hepatitis B and C] and autoimmune hepatitis); alcoholic and non-alcoholic steatohepatitis; hemochromatosis; Wilson's disease; hepatotoxicity; cirrhosis; acute and chronic liver failure; and combinations thereof.

In certain embodiments, the pruritus is associated with a cholestatic disorder (e.g., cholestasis or primary biliary cirrhosis), or cholestatic pruritus. In further embodiments, the pruritus is associated with cirrhosis. In still further embodiments, the pruritus is associated with liver failure. In additional embodiments, the pruritus is associated with hepatitis (e.g., chronic hepatitis B, chronic hepatitis C or autoimmune hepatitis). In other embodiments, the pruritus is induced by a drug or toxin that causes a hepato-biliary disease or liver damage/injury (hepatotoxicity).

One or more NK-1 antagonists can be used to treat acute or chronic pruritus associated with a condition described herein. In some embodiments, the NK-1 antagonist is or comprises a selective NK-1 antagonist. Non-limiting examples of NK-1 antagonists include aprepitant (L-754030 or MK-869), fosaprepitant (L-758298), befetupitant, casopitant (GW-679769), dapitant (RPR-100893), ezlopitant (CJ-11974), lanepitant (LY-303870), maropitant (CJ-11972), netupitant, nolpitantium (SR-1403:33), orvepitant (GW-823296), rolapitant, serlopitant, tradipitant (VLY-686 or LY-686017), vestipitant (GW-597599), vofopitant (GR-205171), hydroxyphenyl propamidobenzoic acid, maltooligosaccharides maltotetraose and maltopentaose), spantides (e.g., spantide I and II), AV-608, AV-818, AZD-2624, 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LV-686017, M516102, MDL-105212, NIP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and analogs, derivatives, prodrugs, metabolites and salts thereof. In certain embodiments, the NK-1 antagonist is or comprises serlopitant (described in greater detail below), or a pharmaceutically acceptable salt, solvate, hydrate, clathrate, polymorph, prodrug or metabolite thereof.

The therapeutically effective amount and the frequency of administration of, and the length of treatment with, the NK-1 antagonist (e.g., serlopitant) to treat acute or chronic pruritus associated with a medical condition may depend on various factors, including the nature and the severity of the pruritus or the medical condition, the potency of the NK-1 antagonist, the mode of administration, the age, the body weight, the general health, the gender and the diet of the subject, and the response of the subject to the treatment, and can be determined by the treating physician. In some embodiments, a therapeutically effective amount of the NK-1 antagonist (e.g., serlopitant) for the treatment of acute or chronic pruritus associated with a condition described herein is about 0.1-200 mg, 0.1-150 mg, 0.1-100 mg, 0.1-50 mg, 0.1-30 mg, 0.5-20 mg, 0.5-10 mg or 1-10 mg (e.g., per day or per dose), or as deemed appropriate by the treating physician, which can be administered in a single dose or in divided doses. In certain embodiments, the therapeutically effective dose (e.g., per day or per dose) of the NK-1 antagonist (e.g., serlopitant) for treating acute or chronic pruritus associated with a condition described herein is about 0.1-1 mg (e.g., about 0.1 mg, 0.5 mg or 1 mg), about 1-5 mg (e.g., about 1 mg, 2 mg, 3 mg, 4 mg or 5 mg), about 5-10 mg (e.g., about 5 mg, 6 mg, 7 mg, 8 mg, 9 mg or 10 mg), about 10-20 mg (e.g., about 10 mg, 15 mg or 20 mg), about 20-30 mg (e.g., about 20 mg, 25 mg or 30 mg), about 30-40 mg (e.g., about 30 mg, 35 mg or 40 mg), about 40-50 mg (e.g., about 40 mg, 45 mg or 50 mg), about 50-100 mg (e.g., about 50 mg, 60 mg, 70 mg, 80 mg, 90 mg or 100 mg), about 100-150 mg (e.g., about 100 mg, 125 mg or 150 mg), or about 150-200 mg (e.g., about 150 mg, 175 mg or 200 mg). In some embodiments, the therapeutically effective dose of the NK-1 antagonist (e.g., serlopitant) is administered one or more (e.g., two, three or more) times a day, or once every two or three days, or once, twice or thrice a week, or as deemed appropriate by the treating physician. In certain embodiments, the therapeutically effective dose of the NK-1 antagonist (e.g., serlopitant) is administered once daily. In further embodiments, the therapeutically effective dose of the NK-1 antagonist (e.g., serlopitant) is about 0.5-5 mg, 1-5 mg or 5-10 mg (e.g., about 0.5 mg, 1 mg, 5 mg or 10 mg) once daily. In certain embodiments, the therapeutically effective dose of the NK-1 antagonist (e.g., serlopitant) is about 5 mg once daily.

The NK-1 antagonist (e.g., serlopitant) can also be dosed in an irregular manner. For example, the NK-1 antagonist can be administered once, twice or thrice in a period of two weeks, three weeks or a month in an irregular manner. Furthermore, the NK-1 antagonist (e.g., serlopitant) can be taken pro re rata (as needed). For instance, the NK-1 antagonist can be administered 1, 2, 3, 4, 5 or more times, whether in a regular or irregular manner, until pruritus improves. Once relief from itch is achieved, dosing of the NK-1 antagonist can optionally be discontinued. If pruritus returns, administration of the NK-1 antagonist, whether in a regular or irregular manner, can be resumed. The appropriate dosage of, frequency of dosing of and length of treatment with the NK-1 antagonist can be determined by the treating physician.

In some embodiments, the NK-1 antagonist (e.g., serlopitant) is administered under a chronic dosing regimen for the treatment of chronic pruritus associated with a condition described herein. In certain embodiments, a therapeutically effective amount of the NK-1 antagonist (e.g., serlopitant) is administered over a period of at least about 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months). Administration of the NK-1 antagonist (e.g., serlopitant) over a period of less than about 6 weeks (e.g., for about 1 week, 2 weeks, 3 weeks, 4 weeks or 5 weeks) can be regarded as treatment of acute pruritus.

The NK-1 antagonist (e.g., serlopitant) can also be used prophylactically to prevent pruritus (e.g., acute pruritus). For instance, the NK-1 antagonist can be taken prior to exposure to an agent or substance that may cause pruritus, such as an allergen (which may cause, e.g., allergic contact dermatitis and pruritus), a chemical or material (which may cause, e.g., irritant contact dermatitis and pruritus), water (which may cause, e.g., aquagenic dermatitis or water urticaria and pruritus), or sunlight (which may cause, e.g., phototoxic dermatitis or solar urticaria and pruritus). As an example, a subject can take an NK-1 antagonist before be goes through an area containing poison ivy or poison oak. The prophylactically effective amount of an NK-1 antagonist (e.g., serlopitant) can be any therapeutically effective amount of the NK-1 antagonist described herein.

The NK-1 antagonist (e.g., serlopitant) can be administered via any suitable route. Potential routes of administration of the NK-1 antagonist include without limitation oral, parenteral (including intramuscular, subcutaneous, intradermal, intravascular, intravenous, intraarterial, intramedullary and intrathecal), intracavitary, intraperitoneal, and topical (including dermal/epicutaneous, transdermal, mucosal, transmucosal, intranasal [e.g., by nasal spray or drop], intraocular [e.g., by eye drop], pulmonary [e.g., by oral or nasal inhalation], buccal, sublingual, rectal and vaginal). In certain embodiments, the NK-1 antagonist (e.g., serlopitant) is administered orally (e.g., as a capsule or tablet, optionally with an enteric coating). In other embodiments, the NK-1 antagonist (e.g., serlopitant) is administered parenterally (e.g., intravenously, subcutaneously or intradermally). In further embodiments, the NK-1 antagonist (e.g., serlopitant) is administered topically (e.g., dermally/epicutaneously, transdermally, mucosally, transmucosally, buccally or sublingually).

For the treatment of chronic pruritus associated with a condition described herein, in some embodiments the NK-1 antagonist (e.g., serlopitant) is administered in a dose of about 0.5, 1, 5 or 10 rug orally (e.g., as a tablet) once daily for at least about 6 weeks, 2 months, 10 weeks, 3 months, 4 months, 5 months, 6 months, 1 year, 1.5 years, 2 years, 3 years or longer. The disclosure specifically discloses each of the 44 possible combinations of dose and treatment length. In certain embodiments, the NK-1 antagonist (e.g., serlopitant) is administered in a dose of about 5 mg orally (e.g., as a tablet) once daily for at least about 6 weeks, 2 months, 3 months or 6 months.

The NK-1 antagonist (e.g., serlopitant) can be administered at any time convenient to the patient. However, NK-1 antagonists may cause drowsiness. To avoid or minimize drowsiness or dizziness during the day, the NK-1 antagonist (e.g., serlopitant) can be administered shortly before the patient goes to bed. Moreover, use of the NK-1 antagonist (e.g., serlopitant) at night can aid with sleep and decrease nocturnal itch and scratching. Accordingly, in certain embodiments the NK-1 antagonist (e.g., serlopitant) is administered at bedtime (e.g., once daily at bedtime). The NK-1 antagonist can also be administered at any appropriate time during the day or awake hours (e.g., in the morning).

In additional embodiments, the NK-1 antagonist (e.g., serlopitant) is administered without food. In some embodiments, the NK-1 antagonist (e.g., serlopitant) is administered at least about 1 or 2 hours before or after a meal. In certain embodiments, the NK-1 antagonist (e.g., serlopitant) is administered at least about 2 hours after an evening meal. The NK-1 antagonist can also be taken substantially concurrently with food (e.g., within about 0.5, 1 or 2 hours before or after a meal, or with a meal).

In some embodiments where a more rapid establishment of a therapeutic level of the NK-1 antagonist (e.g., serlopitant) is desired, the NK-1 antagonist is administered under a dosing schedule in which a loading dose is administered, followed by (i) one or more additional loading doses and then one or more therapeutically effective maintenance doses, or (ii) one or more therapeutically effective maintenance doses without an additional loading dose, as deemed appropriate by the treating physician. A loading dose of a drug is typically larger (e.g., about 1.5, 2, 3, 4 or 5 times larger) than a subsequent maintenance dose and is designed to establish a therapeutic level of the drug more quickly. The one or more therapeutically effective maintenance doses can be any therapeutically effective dose described herein. In certain embodiments, the loading dose is about three times greater than the maintenance dose. In some embodiments, a loading dose of the NK-1 antagonist (e.g., serlopitant) is administered, followed by administration of a maintenance dose of the NK-1 antagonist after an appropriate time (e.g., after about 12 or 24 hr) and thereafter for the duration of therapy—e.g., a loading dose of the NK-1 antagonist is administered on day 1 and a maintenance dose is administered on day 2 and thereafter for the duration of therapy. In some embodiments, the NK-1 antagonist (e.g., serlopitant) is administered in a loading, dose of about 1.5, 3, 15 or 30 mg (e.g., 3×about 0.5, 1, 5 or 10 mg) orally (e.g., as a tablet) on day 1, followed by a maintenance dose of about 0.5, 1, 5 or 10 mg orally (e.g., as a tablet) once daily, optionally at bedtime, for at least about 2 weeks, 1 month (4 weeks), 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months). In certain embodiments, the NK-1 antagonist (e.g., serlopitant) is administered in a loading dose of about 15 mg (e.g., 3×about 5 mg) orally (e.g., as a tablet) on day 1, followed by a maintenance dose of about 5 mg orally (e.g., as a tablet) once daily, optionally at bedtime, for at least about 2 weeks, 1 month, 6 weeks, 2 months, 3 months, 6 months, 1 year, 1.5 years, 2 years, 3 years or longer (e.g., at least about 6 weeks, 2 months, 3 months or 6 months).

In other embodiments, a first loading dose of the NK-1 antagonist (e.g., serlopitant) is administered on day 1, a second loading dose is administered on day 2, and a maintenance dose is administered on day 3 and thereafter for the duration of therapy. In certain embodiment, the first loading dose is about three times greater than the maintenance dose, and the second loading dose is about two times greater than the maintenance dose.

In some embodiments, one or more additional antipruritic or therapeutic agents in addition to an NK-1 antagonist (e.g., serlopitant) are administered for the treatment of acute or chronic pruritus associated with a medical condition described herein, or/and the medical condition itself.

An NK-1 antagonist (e.g., serlopitant), optionally in combination with one or more additional antipruritic or therapeutic agents, can be used to treat pruritus of any degree of severity (e.g., mild, moderate or severe), pruritus associated with a medical condition of any degree of severity (e.g., mild, moderate or severe), or the medical condition itself of any degree of severity (e.g., mild, moderate or severe). As a non-limiting example, an NK-1 antagonist (e.g., serlopitant), optionally in combination with one or more additional antipruritic or therapeutic agents, can be used to treat pruritus of any degree of severity associated with a dermatological condition, pruritus associated with a dermatological condition of any degree of severity, or the dermatological condition itself of any degree of severity, such as treating moderate to severe pruritus associated with dermatitis (e.g., atopic dermatitis), psoriasis (e.g., plaque psoriasis) or urticaria (e.g., idiopathic urticaria); treating pruritus associated with moderate to severe dermatitis (e.g., atopic dermatitis), psoriasis (e.g., plaque psoriasis) or urticaria (e.g., idiopathic urticaria); or treating moderate to severe dermatitis (e.g., atopic dermatitis), psoriasis (e.g., plaque psoriasis) or urticaria (e.g., idiopathic urticaria). It should be noted that the degree of severity of pruritus does not necessarily correlate with the degree of severity of a medical condition (e.g., a dermatological condition). For instance, patients with mild psoriasis (e.g, plaque psoriasis) can have severe pruritus.

An NK-1 antagonist (e.g., serlopitant) can be used to treat other symptoms or complications of a medical condition described herein besides pruritus, or can be used to treat the medical condition itself. For example, the NK-1 antagonist (e.g., serlopitant) can be administered to slow the progression or to reduce the severity of the medical condition, to improve the health or/and the function of a tissue or organ (e.g., skin or liver), to decrease the number, frequency, area, extent or/and severity of skin symptoms (e.g., skin lesions, rashes, flares, nodules, papules, plaques, blisters and wheals), or to improve wound healing (e.g., reduce wound surface area and reduce the number and size of open sores), or any combination thereof, wherein the additional therapeutic benefits may or may not result from reduction in itch and scratching (e.g., they may be due to the NK-1 antagonist's anti-inflammatory, anti-proliferative or/and anti-metastatic effects). All embodiments described herein for the treatment of acute or chronic pruritus associated with a medical condition using an NK-1 antagonist (e.g., serlopitant), including without limitation all embodiments relating to the therapeutically effective amount of, the frequency of dosing of and the length of treatment with the NK-1 antagonist, also apply to the treatment of other symptoms or complications of a medical condition, or to the treatment of the medical condition itself, using an NK-1 antagonist (e.g., serlopitant).

The disclosure provides for the use of an NK-1 antagonist (e.g., serlopitant) in the preparation of a medicament for the treatment of acute or chronic pruritus associated with any medical condition described herein, or for the treatment of any medical condition described herein, optionally in combination with the use of any one or more other antipruritic or therapeutic agents described herein in the preparation of a medicament for the treatment of acute or chronic pruritus associated with any medical condition described herein, or for the treatment of any medical condition described herein. The disclosure further provides an NK-1 antagonist (e.g., serlopitant) for use in the treatment of acute or chronic pruritus associated with any medical condition described herein, or in the treatment of any medical condition described herein, optionally in combination with any one or more other antipruritic or therapeutic agents described herein for use in the treatment of acute or chronic pruritus associated with any medical condition described herein, or in the treatment of any medical condition described herein.

### Neurokinin-1 Antagonists

As described above, the disclosure provides for the use of one or more NK-1 antagonists in the treatment of acute or chronic pruritus associated with a condition described herein. In some embodiments, the NK-1 antagonist is or comprises a selective NK-1 antagonist. Examples of NK-1 antagonists include without limitation aprepitant (L-7540:30 or MK-869), fosaprepitant (L-758298), befetupitant, casopitant (GW-679769), dapitant (RPR-100893), ezlopitant (CJ-11974), lanepitant (LY-303870), maropitant (CJ-11972), netupitant, nolpitantium (SR-140333), orvepitant (GW-823296), rolapitant, serlopitant, tradipitant (VLY-686 or LY-686017), vestipitant (GW-597599), vofopitant (GR-205171), hydroxyphenyl propamidobenzoic acid, maltooligosaccharides (e.g., maltotetraose and maltopentaose), spantides (e.g., spantide I and II), AV-608, AV-818, AZD-2624, BIT 1149 CL, CGP-49823, CJ-1749:3, CP-96345, CP-99994, CP-122721, DIN-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and analogs, derivatives, prodrugs, metabolites and salts thereof.

In some embodiments, the NK-1 antagonist is or includes serlopitant, or a pharmaceutically acceptable salt, solvate, hydrate, clathrate, polymorph, prodrug or metabolite thereof. In further embodiments, the NK-1 antagonist is or includes aprepitant or fosaprepitain (a prodrug of aprepitant), or a pharmaceutically acceptable salt, solvate, hydrate, clathrate, polymorph, prodrug or metabolite thereof.

In additional embodiments, the NK-1 antagonist is or includes befetupitant, or a pharmaceutically acceptable salt, solvate, hydrate, clathrate, polymorph, prodrug or metabolite thereof. In further embodiments, the NK-1 antagonist is or includes casopitant, or a pharmaceutically acceptable salt, solvate, hydrate, clathrate, polymorph, prodrug or metabolite thereof. In still further embodiments, the NK-1 antagonist is or includes dapitant, or a pharmaceutically acceptable salt, solvate, hydrate, clathrate, polymorph, prodrug or metabolite thereof. In yet further embodiments, the NK-1 antagonist is or includes ezlopitant, or a pharmaceutically acceptable salt, solvate, hydrate, clathrate, polymorph, prodrug or metabolite thereof. In other embodiments, the NK-1 antagonist is or includes lanepitant, or a pharmaceutically acceptable salt, solvate, hydrate, clathrate, polymorph, prodrug or metabolite thereof. In still other embodiments, the NK-1 antagonist is or includes maropitant, or a pharmaceutically acceptable salt, solvate, hydrate, clathrate, polymorph, prodrug or metabolite thereof. In yet other embodiments, the NK-1 antagonist is or includes netupitant, or a pharmaceutically acceptable salt, solvate, hydrate, clathrate, polymorph, prodrug or metabolite thereof.

In further embodiments, the NK-1 antagonist is or includes nolpitantium, or a pharmaceutically acceptable salt, solvate, hydrate, clathrate, polymorph, prodrug or metabolite thereof. In still further embodiments, the NK-1 antagonist is or includes orvepitant, or a pharmaceutically acceptable salt, solvate, hydrate, clathrate, polymorph, prodrug or metabolite thereof. In yet further embodiments, the NK-1 antagonist is or includes rolapitant, or a pharmaceutically acceptable salt, solvate, hydrate, clathrate, polymorph, prodrug or metabolite thereof. In other embodiments, the NK-1 antagonist is or includes tradipitant, or a pharmaceutically acceptable salt, solvate, hydrate, clathrate, polymorph, prodrug or metabolite thereof. In still other embodiments, the NK-1 antagonist is or includes vestipitant, or a pharmaceutically acceptable salt, solvate, hydrate, clathrate, polymorph, prodrug or metabolite thereof. In yet other embodiments, the NK-1 antagonist is or includes vofopitant, or a pharmaceutically acceptable salt, solvate, hydrate, clathrate, polymorph, prodrug or metabolite thereof.

In additional embodiments, the NK-1 antagonist is or includes DNK-333, or a pharmaceutically acceptable salt, solvate, hydrate, clathrate, polymorph, prodrug or metabolite thereof. In further embodiments, the NK-1 antagonist is or includes SCH-900978, or a pharmaceutically acceptable salt, solvate, hydrate, clathrate, polymorph, prodrug or metabolite thereof.

In some embodiments, the NK-1 antagonist is not, or does not include, aprepitant or fosaprepitant for the treatment of pruritus associated with, e.g., atopic dermatitis (AD), prurigo nodularis (PN), urticaria, CTCL, epidermolysis bullosa, a burn or a hepato-biliary disease. In further embodiments, the NK-1 antagonist is not, or does not include, a piperazine- or piperidine-containing compound disclosed in EP 1,295,599 A1 for the treatment of pruritus associated with, e.g., AD. In still further embodiments, the NK-1 antagonist is not, or does not include, a substituted acrylamide compound disclosed in WO 2010/097381 A1 for the treatment of pruritus associated with, e.g., AD, PN, urticaria, CTCL or a Hepato-biliary disease. In additional embodiments, the NK-1 antagonist is not, or does not include, a substituted pyrrolo[1,2-a]piperazine or pyrrolo[1,2-a][1,4]diazepine compound disclosed in WO 2013/124286 A1 for the treatment of pruritus associated with, e.g., AD, PN, urticaria, CTCL or a hepato-biliary disease. In other embodiments, the NK-1 antagonist is not, or does not include, orvepitant for the treatment of pruritus associated with, e.g., a burn. In still other embodiments, the NK-1 antagonist is not, or does not include, tradipitant for the treatment of pruritus associated with, e.g., AD.

### Description of Serlopitant

Serlopitant is a potent and highly selective antagonist of neurokinin-1 (also called substance P receptor). By binding to and not activating NK-1, serlopitant can inhibit actions of substance P, including the transmission of itch from the skin to the CNS, mediation of inflammation, stimulation of growth of cancer cells, and promotion of metastasis of cancer cells.

Serlopitant has the structure shown below. The IUPAC name for serlopitant is 3-[(aR,4R,5S,7aS)-5-[(1R)-1-[3,5-bis(trifluoromethyl)phenyl]ethoxy]-4-(4-fluorophenyl)-1,3,3a,4,5,6,7,7a-octahydroisoindol-2-yl]cyclopent-2-en-1-one. The USAN name for serlopitant is 3-[(3aR,4R,5S,7aS)-5-[(1R)-1-[3,5-bis(trifluoromethyl)phenyl]ethoxy]-4-(4-fluorophenyl)octahydro-2H-isoindol-2-yl]cyclopent-2-en-1-one. The disclosure also encompasses all stereoisomers of serlopitant, including both enantiomers and all diastereomers of serlopitant in substantially pure form and mixtures of both enantiomers (including a racemic mixture) and mixtures of two or more diastereomers of serlopitant in any ratio. The disclosure further encompasses all isotopically enriched forms of serlopitant, including without limitation those enriched in the content of ²H (deuterium), ¹³C, ¹⁵N, ¹⁷O or ¹⁸O, or any combination thereof, at one or more, or all, instances of the corresponding atom(s). Moreover, the disclosure encompasses any and all salt forms of serlopitant. Various methods of synthesizing serlopitant are known in the art. See, e.g., Jiang et al., J. Med. Chem., 52:3039-3046 (2009); U.S. Pat. No. 7,544,815 by Kuethe et al.; and U.S. Pat. No. 7,217,731 by Bunda et al.

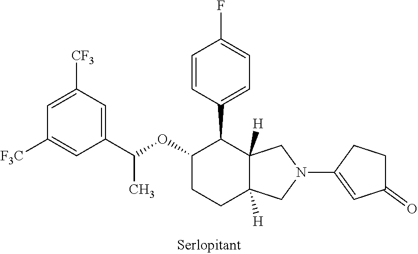

Whether as a free base or a salt, serlopitant can exist unsolvated or unhydrated, or solvated or hydrated. Solvated forms of serlopitant can be formed with a pharmaceutically acceptable solvent, such as water or ethanol. In certain embodiments, serlopitant, whether as a free base or a salt, is used substantially unhydrated.

The disclosure also encompasses polymorphs (crystalline forms) of serlopitant. Examples of polymorphs of serlopitant include without limitation anhydrous crystalline Forms I and II of free base serlopitant as disclosed in US Pub. No. 2009/0270477 by Kuethe et al. Form I is characterized by diffraction peaks obtained from X-ray powder diffraction pattern corresponding to d-spacings of 10.4, 9.9, 9.2, 5.5, 5.0, 4.1, 3.9, 3.6 and 3.5 angstroms. Form II is characterized by diffraction peaks obtained from X-ray powder diffraction pattern corresponding to d-spacings of 7.7, 5.3, 4.9, 4.8, 4.6, 4.2, 3.9, 3.8 and 2.8 angstroms. Form I is thermodynamically more stable below 70° C. and is non-hygroscopic under all tested relative humidity conditions. In certain embodiments, serlopitant is used in the form of poly morph Form I.

### Salt Forms of Drug Substances

Drug substances (e.g., NK-1 antagonists such as serlopitant) may exist in a non-salt form (e.g., a free base or a free acid, or having no basic or acidic atom or functional group) or as salts if they can form salts. Drug substances that can form salts can be used in the non-salt form or in the form of pharmaceutically acceptable salts. If a drug has, e.g., a basic nitrogen atom, the drug can form an addition salt with an acid (e.g., a mineral acid [such as HCl, HBr, HI, nitric acid, phosphoric acid or sulfuric acid] or an organic acid [such as a carboxylic acid or a sulfonic acid]). Suitable acids for use in the preparation of pharmaceutically acceptable salts include without limitation acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid. D-gluconic acid, D-glucuronic acid, L-glutamic acid, alpha-oxo-glutaric acid, glycolic acid, hippuric acid, hydrobromic acid, hydrochloric acid, hydroiodic acid, (±)-DL, lactic acid, (+)-L-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, propionic acid, L-pyroglutamic acid, pyruvic acid, saccharic acid, salicylic acid, 4-amino-salicylic acid, sebacic acid, stearic acid, succinic acid, sulfuric acid, tannic acid, (±)-DL-tartaric acid, (+)-L-tartaric acid, thiocyanic acid, p-toluenesulfonic acid, undecylenic acid, and valeric acid.

If a drug has an acidic group (e.g., a carboxyl group), the drug can form an addition salt with a base. Pharmaceutically acceptable base addition salts can be formed with, e.g., metals (e.g., alkali metals or alkaline earth metals) or amines (e.g., organic amines). Non-limiting examples of metals useful as cations include alkali metals (e.g., lithium, sodium, potassium and cesium), alkaline earth metals (e.g., magnesium and calcium), aluminum and zinc. Metal cations can be provided by way of, e.g., inorganic bases, such as hydroxides, carbonates and hydrogen carbonates. Non-limiting examples of organic amines useful for forming base addition salts include chloroprocaine, choline, cyclohexylamine, dibenzylamine, N,N-dibenzylethylenediamine, dicyclohexylamine, diethanolamine, ethylenediamine, N-ethylpiperidine, histidine, isopropylamine, N-methylglucamine, procaine, pyrazine, triethylamine and trimethylamine. Pharmaceutically acceptable salts are discussed in detail in Handbook of Pharmaceutical Salts, Properties, Selection and Use, P. Stahl and C. Wermuth, Eds., Wiley-VCH (2011).

### Pharmaceutical Compositions

To treat acute or chronic pruritus associated with a condition described herein, an NK-1 antagonist (e.g., serlopitant) can be administered alone or in the form of a pharmaceutical composition. In some embodiments, a pharmaceutical composition comprises an NK-1 antagonist (e.g., serlopitant) or a pharmaceutically acceptable salt, solvate, hydrate, clathrate, polymorph, prodrug or metabolite thereof, and one or more pharmaceutically acceptable carriers or excipients. The composition can optionally contain an additional therapeutic agent as described herein. For purposes of the content of a pharmaceutical composition, the terms “therapeutic agent”, “active ingredient”, “active agent” and “drug” encompass prodrugs.

Pharmaceutically acceptable carriers and excipients include pharmaceutically acceptable materials, vehicles and substances. Non-limiting examples of excipients include liquid and solid fillers, diluents, binders, lubricants, glidants, solubilizers, surfactants, dispersing agents, disintegration agents, emulsifying agents, wetting agents, suspending agents, thickeners, solvents, isotonic agents, buffers, pH adjusters, stabilizers, preservatives, antioxidants, antimicrobial agents, antibacterial agents, antifungal agents, absorption-delaying agents, sweetening agents, flavoring agents, coloring agents, adjuvants, encapsulating materials and coating materials. The use of such excipients in pharmaceutical formulations is known in the art. Except insofar as any conventional carrier or excipient is incompatible with the active ingredient, the disclosure encompasses the use of conventional carriers and excipients in formulations containing an NK-1 antagonist (e.g., serlopitant). See, e.g., Remington: The Science and Practice of Pharmacy, 21st Ed., Lippincott Williams &. Wilkins (Philadelphia, Pa. [2005]); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press (Boca Raton, Fla. [2004]).

Proper formulation can depend on various factors, such as the mode of administration chosen. Potential modes of administration of pharmaceutical compositions comprising an NK-1 antagonist (e.g., serlopitant) include without limitation oral, parenteral (including intramuscular, subcutaneous, intradermal, intravascular, intravenous, intraarterial, intramedullary and intrathecal), intracavitary, intraperitoneal, and topical (including dermal/epicutaneous, transdermal, mucosal, transmucosal, intranasal [e.g., by nasal spray or drop], intraocular [e.g., by eye drop], pulmonary [e.g., by oral or nasal inhalation], buccal, sublingual, rectal and vaginal).

As an example, formulations of an NK-1 antagonist (e.g., serlopitant) suitable for oral administration can be presented as, e.g., boluses; tablets, capsules, pills, cachets or lozenges; as powders or granules; as semisolids, electuaries or pastes; as solutions or suspensions in an aqueous liquid or/and a non-aqueous liquid; or as oil-in-water liquid emulsions or water-in-oil liquid emulsions.

Tablets can contain an NK-1 antagonist (e.g., serlopitant) in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal tract and thereby provides a sustained action over a longer time period. In certain embodiments, a tablet comprises an NK-1 antagonist (e.g., serlopitant), mannitol, microcrystalline cellulose, magnesium stearate, silicon dioxide, croscarmellose sodium and sodium lauryl sulfate, and optionally lactose monohydrate, and the tablet is optionally film-coated (e.g., with Opadry®).

Push-fit capsules or two-piece hard gelatin capsules can contain an NK-1 antagonist (e.g., serlopitant) in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, an NK-1 antagonist (e.g., serlopitant) can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions. Dispersible powder or granules of an NK-1 antagonist (e.g., serlopitant) can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

In some embodiments, an NK-1 antagonist (e.g., serlopitant) is contained in an amphiphilic vehicle of a liquid or semi-solid formulation for oral administration which provides improved solubility, stability and bioavailability of the NK-1 antagonist, as described in US Pub. No. 2010/0209496 by Dokou et al. The amphiphilic vehicle contains a solution, suspension, emulsion (e.g., oil-in-water emulsion) or semi-solid mixture of the NK-1 antagonist (e.g., serlopitant) admixed with liquid or/and semi-solid excipients which fills an encapsulated dosage form (e.g., a hard gelatin capsule or a soft gelatin capsule containing a plasticizer [e.g., glycerol or/and sorbitol]). In some embodiments, the amphiphilic vehicle comprises an amphiphilic agent selected from fatty acid esters of glycerol (glycerin), propylene glycol and sorbitol. In certain embodiments, the amphiphilic agent is selected from mono- and di-glycerides of C₈-C₁₂ saturated fatty acids. In further embodiments, the amphiphilic agent is selected from CAPMUL® MCM, CAPMUL® MCM 8, CAPMUL® MCM 10, IMWITOR® 308, IMWITOR® 624, IMWITOR® 742, IMWITOR® 988, CAPRYOL™ PGMC, CAPRYOL™ 90, LAUROGLYCOL™ 90, CAPTEX® 200, CRILL™ 1, CRILL™ 4, PECEOL® and MAISINE™ 35-1. In some embodiments, the amphiphilic vehicle further comprises propylene glycol, a propylene glycol-sparing agent (e.g., ethanol or/and glycerol), or an antioxidant (e.g., butylated hydroxyanisole, butylated hydroxytoluene, propyl gallate or/and sodium sulfite), or any combination or all thereof. In additional embodiments, the amphiphilic vehicle contains on a weight basis about 0.1-5% of the NK-1 antagonist (e.g., serlopitant), about 50-90% of the amphiphilic agent, about 5-40% of propylene glycol, about 5-20% of the propylene glycol-sparing agent, and about 0.01-0.5% of the antioxidant.

An NK-1 antagonist (e.g., serlopitant) can also be formulated for parenteral administration by injection or infusion. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain an NK-1 antagonist (e.g., serlopitant) along with excipients such as an antioxidant, a buffer, a bacteriostat and solutes that render the formulation isotonic with the blood of the subject. Aqueous or non-aqueous sterile suspensions can contain an NK-1 antagonist (e.g., serlopitant) along with excipients such as a suspending agent and a thickening agent, and optionally a stabilizer and an agent that increases the solubility of the NK-1 antagonist to allow for the preparation of a more concentrated solution or suspension.

For topical administration, an NK-1 antagonist (e.g., serlopitant) can be formulated as, e.g., a buccal or sublingual tablet or pill. Advantages of a buccal or sublingual tablet or pill include avoidance of first-pass metabolism and circumvention of gastrointestinal absorption. A buccal or sublingual tablet or pill can also be designed to provide faster release of the NK-1 antagonist for more rapid uptake of it into systemic circulation. In addition to a therapeutically effective amount of the NK-1 antagonist (e.g., serlopitant), the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, an NK-1 antagonist (e.g., serlopitant) can also be formulated for intranasal administration. The nasal mucosa provides a big surface area, a porous endothelium, a highly vascular subepithelial layer and a high absorption rate, and hence allows for high bioavailability. Moreover, intranasal administration avoids first-pass metabolism and can introduce a significant concentration of the NK-1 antagonist to the central nervous system. An intranasal formulation can comprise an NK-1 antagonist (e.g., serlopitant) along with excipients such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer.

An additional mode of topical administration of an NK-1 antagonist (e.g., serlopitant) is pulmonary, including by oral inhalation and nasal inhalation. The lungs serve as a portal to the systemic circulation. Advantages of pulmonary drug delivery include, for example: 1) avoidance of first pass hepatic metabolism; 2) fast drug action; 3) large surface area of the alveolar region for absorption, high permeability of the lungs (thin air-blood barrier), and profuse vasculature of the airways; 4) reduced extracellular enzyme levels compared to the gastrointestinal tract clue to the large alveolar surface area; and 5) smaller doses to achieve equivalent therapeutic effect compared to other oral routes, and hence reduced systemic side effects. An advantage of oral inhalation over nasal inhalation includes deeper penetration/deposition of the drug into the lungs. Oral or nasal inhalation can be achieved by means of, e.g., a metered-dose inhaler, a dry powder inhaler or a nebulizer.

Other suitable topical formulations and dosage forms include without limitation ointments, creams, gels, lotions, pastes and the like, as described in Remington: The Science and Practice of Pharmacy, 21st Ed., Lippincott Williams & Wilkins (Philadelphia, Pa. [2005]). Ointments are semi-solid preparations that are typically based on petrolatum or a petroleum derivative. Creams are viscous liquids or semi-solid emulsions, either oil-in-water or water-in-oil. Cream bases are water-washable, and contain an oil phase, an emulsifier and an aqueous phase. The oil phase, also called the “internal” phase, generally comprises petrolatum and a fatty alcohol (e.g., cetyl or stearyl alcohol). The aqueous phase typically, although not necessarily, exceeds the oil phase in volume, and usually contains a humectant. The emulsifier in a cream formulation is generally a non-ionic, anionic, cationic or amphoteric surfactant. Gels are semi-solid, suspension-type systems. Single-phase gels contain organic macromolecules (polymers) distributed substantially uniformly throughout the carrier liquid, which is typically aqueous but can also contain an alcohol (e.g., ethanol or isopropanol) and optionally an oil. Lotions are preparations to be applied to the skin surface without friction, and are typically liquid or semi-liquid preparations in which solid particles, including the active agent, are present in a water or alcohol base. Lotions are usually suspensions of finely divided solids and typically contain suspending agents to produce better dispersion as well as compounds useful for localizing and holding the active agent in contact with the skin. Pastes are semi-solid dosage forms in which the active agent is suspended in a suitable base. Depending on the nature of the base, pastes are divided between fatty pastes or those made from single-phase aqueous gels.

Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g. menthol), opacifiers, fragrances and colorants. For an active agent having a low rate of permeation through the skin or mucosal tissue, a topical formulation can contain a permeation enhancer to increase the permeation of the active agent through the skin or mucosal tissue. A topical formulation can also contain an irritation-mitigating excipient that reduces any irritation to the skin or mucosa caused by the active agent, the permeation enhancer or any other component of the formulation.

In some embodiments, NK-1 antagonist (e.g., serlopitant) is delivered from a sustained-release composition. As used herein, the term “sustained-release composition” encompasses sustained-release, prolonged-release, extended-release, slow-release and controlled-release compositions, systems and devices. Use of a sustained-release composition can have benefits, such as an improved profile of the amount of the drug or an active metabolite thereof delivered to the target site(s) over a time period, including delivery of a therapeutically effective amount of the drug or an active metabolite thereof over a prolonged time period. In certain embodiments, the sustained-release composition delivers the NK-1 antagonist over a period of at least about 1 day, 2 days, 3 days, 1 week, 2 weeks, 3 weeks, 1 month, 2 months, 3 months or longer. In some embodiments, the sustained-release composition is a drug-encapsulation system, such as, e.g., nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium polyacrylate), and other homopolymers and copolymers having a large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

In some embodiments, the sustained-release composition is formulated as polymeric nanoparticles or microparticles, wherein the polymeric particles can be delivered, e.g., by injection or from an implant. In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly (L-lactic acid-co-D,L-2-hydroxyoctanoic acid)]. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

For a delayed or sustained release of an NK-1 antagonist (e.g., serlopitant), a composition can also be formulated as a depot that can be implanted in or injected into a subject, e.g., intramuscularly or subcutaneously. A depot formulation can be designed to deliver the NK-1 antagonist over a longer period of time, e.g., over a period of at least about 1 week, 2 weeks, 3 weeks, 1 month, 6 weeks, 2 months, 3 months or longer. For example, the NK-1 antagonist can be formulated with a polymeric material, a hydrophobic material (e.g., as an emulsion in an oil) or/and an ion-exchange resin, or as a sparingly soluble derivative (e.g., a sparingly soluble salt).

In addition, pharmaceutical compositions comprising an NK-1 antagonist (e.g., serlopitant) can be formulated as, e.g., liposomes, micelles, microparticles, microspheres or nanoparticles, whether or not designed for sustained release.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

The compositions can be presented in unit dosage form as a single dose wherein all active and inactive ingredients are combined in a suitable system, and components do not need to be mixed to form the composition to be administered. The unit dosage form can contain an effective dose, or an appropriate fraction thereof, of the NK-1 antagonist (e.g., serlopitant). Representative examples of a unit dosage form include a tablet, capsule, or pill for oral administration.

Alternatively, the compositions can be presented as a kit, wherein the active ingredient, excipients and carriers (e.g., solvents) are provided in two or more separate containers (e.g., ampules, vials, tubes, bottles or syringes) and need to be combined to form the composition to be administered. The kit can contain instructions for preparing and administering the composition (e.g., a solution to be injected intravenously).

A kit can contain all active and inactive ingredients in unit dosage form or the active ingredient and inactive ingredients in two or more separate containers, and can contain instructions for using the pharmaceutical composition to treat pruritus or a pruritus-associated condition.

### Topical Compositions Comprising a Therapeutic Agent Such as an NK-1 Antagonist

Topical formulations for application to the skin or mucosa can be useful for treatment of conditions of the upper skin or mucosal layers and for transdermal or transmucosal administration of an active agent to the local tissue underlying the skin or mucosa and, if desired, into the blood for systemic distribution. Advantages of topical administration can include avoidance of first-pass metabolism, circumvention of gastrointestinal absorption, delivery of an active agent with a relatively short biological half-life, more controlled release of the active agent, administration of a more uniform plasma dosing of the active agent, less side effects, and improvement in user compliance.

In general and in addition to the disclosure on topical formulations described elsewhere herein, compositions suitable for topical administration include without limitation liquid or semi-liquid preparations such as sprays, gels, liniments, lotions, oil-in-water or water-in-oil emulsions such as creams, foams, ointments and pastes, and solutions or suspensions such as drops (e.g., eye drops, nose drops and ear drops). In some embodiments, a topical composition comprises an active agent dissolved, dispersed or suspended in a carrier. The carrier can be in the form of, e.g., a solution, a suspension, an emulsion, an ointment or a gel base, and can contain, e.g., petrolatum, lanolin, a wax (e.g., bee wax), mineral oil, a long-chain alcohol, polyethylene glycol or polypropylene glycol, a diluent (e.g., water or/and an alcohol [e.g., ethanol or propylene glycol]?, an emulsifier, a stabilizer or a thickening agent, or any combination thereof. A topical composition can include, or a topical formulation can be administered by means of, e.g., a transdermal patch, a microneedle patch or an iontophoresis device. A transdermal patch can contain, e.g., a microporous membrane made of a suitable material (e.g., cellulose nitrate or acetate, propylene or a polycarbonate), a skin adhesive and backing material. A topical composition can deliver the active agent transdermally or transmucosally via a concentration gradient or an active mechanism (e.g., iontophoresis).

To enhance the penetration of a small-molecule therapeutic or antipruritic agent (e.g., an NK-1 antagonist) into o/land across the skin or mucosa, a chemical penetration/permeation enhancer (CPE) can be mixed with the therapeutic agent for topical administration. Non-limiting examples of CPEs include hydrocarbons (e.g., alkanes and alkenes [e.g., squalene]), terpenes (e.g., D-limonene, carvone and anise oil), alcohols and fatty alcohols (e.g., ethanol, isopropyl alcohol, pentanol, lauryl alcohol, oleyl alcohol, benzyl alcohol, propylene glycol, dipropylene glycol, polyethylene glycol and glycerol), fatty acids (e.g, valeric acid, lauric acid, oleic acid and linoleic acid), esters, fatty alcohol esters and fatty acid esters (e.g., ethyl acetate, isopropyl myristate, isopropyl palmitate, methyl oleate, ethyl oleate, triacetin and pentadecalactone), hydroxyl-containing esters, fatty alcohol esters and fatty acid esters (e.g., lauryl lactate, glyceryl/glycerol monolaurate, glycerol monoleate [mono-olein], sorbitan oleate and octyl salicylate), amities (e.g., diethanolamine and triethanolamine), amides, fatty amine amides and fatty acid amides (e.g., urea, dimethylformamide, dimethylacetamide, diethyltoluamide, N-lauroyl sarcosine, 1-dodecylazacycloheptane-2-one [Azone®], Azone-related compounds, and pyrrolidone compounds), sulfoxides (e.g., dimethyl sulfoxide), ionic and non-ionic surfactants (e.g., cetyltrimethylammonium bromide, sodium laurate, Brij®, Tween® and sodium cholate), phospholipids (e.g., lecithin), ginsenosides and those described elsewhere herein. US Pub. 2007/0269379 provides an extensive list of CPEs.

In certain embodiments, the CPE includes a surfactant. In some embodiments, the CPE includes two or more surfactants, such as a non-ionic surfactant (e.g., sorbitan monolaurate or N-lauroyl sarcosine) and an ionic surfactant (e.g., an anionic surfactant, such as sodium lauroyl sarcosinate). In further embodiments, the CPE includes a surfactant (e.g., an anionic surfactant, such as sodium laureth sulfate [sodium lauryl ether sulfate]) and an aromatic compound (e.g., 1-phenylpiperazine). Such combinations of CPEs can greatly enhance penetration of drug(s) into or/and through the skin or mucosa with a low potential for skin or mucosal irritation. In additional embodiments, the CPE includes an organic sulfoxide and a compound selected from fatty acids, fatty acid esters and Azone-related compounds.

To enhance the penetration of a polypeptide (e.g., a peptide or a protein) into or/and across the skin or mucosa, alternative to or in addition to a chemical penetration enhancer, a transdermal peptide enhancer (TPE) can be mixed with the polypeptide for topical administration. TPEs include cell-penetrating peptides (CPPs) and transdermal-enhanced peptides (TEPs, also called skin-penetrating peptides [SPPs]). CPPs may be more polar or charged (e.g., positively charged) than TEPs/SPPs. Non-limiting examples of CPPs for transdermal or transmucosal administration include polyarginine homopolymers (e.g., those comprising 6 to 15 arginine residues), arginine-rich CPPs (e.g., the HIV-1 trans-activator of transcription [TAT] peptide, the IMT-P8 peptide and low molecular weight protamine [LMWP]), magainins (e.g., magainin 2), penetratin, Pep-1, the peptide for ocular delivery (POD, which is also capable of penetrating through non-ocular tissues such as the skin), transportan, the WLR (name) peptide and the YARA (name) peptide. Examples of TEPs/SPPs for transdermal or transmucosal administration include without limitation the dermis-localizing peptide (DLP), the linear peptide-12 (LP-12), the skin-penetrating and cell-entering (SPACE) peptide, the T2 peptide, the TD-1 peptide, the TD-34 peptide, and the TDN (name) peptide. A CPP or/and an SPP can be used, or a TPE can be a CPP directly or indirectly linked to an SPP, such as TD-1 linked to polyarginine (e.g., octa-arginine). The polypeptide/TPE complex can diffuse passively through the skin or mucosa down a concentration gradient, even if the complex is charged (has no net charge or has a net charge). If the complex is charged (e.g., the polypeptide is negatively charged and the TPE [e.g., a CPP] is positively charged), translocation of the complex through the skin or mucosa can be facilitated by, e.g, iontophoresis. The TPE may also enhance the aqueous solubility or/and the stability of the polypeptide. The polypeptide solution can be prepared with a solvent that also functions as a CPE, such as ethanol in an aqueous ethanol solution.

In some embodiments, a polypeptide is transdermally or transmucosally administered with a CPP (e.g., a polyarginine such as nona-arginine) or a TEP/SPP (e.g., the SPACE peptide) and without a CPE (other than an alcohol that may be used to prepare the polypeptide solution, such as ethanol). In other embodiments, a polypeptide is transdermally or transmucosally administered with a CPP (e.g., a polyarginine such as nova-arginine) or a TEP/SPP (e.g., die SPACE peptide), and with a CPE (e.g., a fatty acid such as oleic acid).

Transdermal or transmucosal delivery of a polypeptide (or small-molecule) drug can also be enhanced by using a tight junction modulator (TJM) alternative to or in addition to a TPE or/and a CPE. TJMs reversibly open tight junctions between cells and thereby facilitate intercellular/paracellular transport of drugs across epithelial barriers. A TPE or a CPE may also disrupt tight junctions. Examples of TJMs that can be mixed with a drug for transdermal or transmucosal delivery of the drug include without limitation chitosans, cloudin-4, the AT1002 peptide, and the zonula occludens toxin (ZOT).

Representative kinds of topical compositions are described below for purposes of illustration.

I. Topical Compositions Comprising a Permeation Enhancer

In some embodiments, a topical composition comprises an NK-1 antagonist (e.g., serlopitant) and a permeation enhancer. The composition can optionally contain an additional therapeutic agent. In certain embodiments, the composition contains the NK-1 antagonist (e.g., serlopitant) in free base form.

The permeation enhancer increases the permeability of the skin or mucosa to the therapeutic agent(s). In certain embodiments, the permeation enhancer is N-lauroyl sarcosine, sodium octyl sulfate, methyl laurate, isopropyl myristate, oleic acid, glyceryl oleate or sodium lauryl sulfoacetate, or any combination thereof. In certain embodiments, the composition contains on a weight/volume (w/v) basis the permeation enhancer in an amount of about 1-20%, 1-1.5%, 1-10% or 1-5%. To enhance further the ability of the therapeutic agent(s) to penetrate the skin or mucosa, the composition can also contain a surfactant, an atone-like compound, an alcohol, a fatty acid or ester, or an aliphatic thiol.

The composition can further contain one or more additional excipients. Suitable excipients include without limitation solubilizers (e.g., C₂-C₈ alcohols), moisturizers or humectants (e.g., glycerol [glycerin], propylene glycol, amino acids and derivatives thereof, polyamino acids and derivatives thereof, and pyrrolidone carboxylic acids and salts and derivatives thereof), surfactants (e.g., sodium laureth sulfate and sorbitan monolaurate), emulsifiers (e.g., cetyl alcohol and stearyl alcohol), thickeners (e.g., methyl cellulose, ethyl cellulose, hydroxymethyl cellulose, hydroxypropyl cellulose, polyvinylpyrrolidone, polyvinyl alcohol and acrylic polymers), and formulation bases or carriers (e.g., polyethylene glycol as an ointment base). As a non-limiting example, the base or carrier of the composition can contain ethanol, propylene glycol and polyethylene glycol (e.g., PEG 300), and optionally an aqueous liquid (e.g., isotonic phosphate-buffered saline).

The topical composition can have any suitable dosage form, such as a solution (e.g., eye drop, nose drop or ear drop), a suspension, an emulsion, a cream, a lotion, a gel, an ointment, a paste, a jelly, a foam, a shampoo, or a spray. In some embodiments, the composition is applied to the skin or mucosa coveting a surface area of about 10-800 cm², 10-400 cm² or 10-200 cm². The composition can deliver the therapeutic agent(s) to the skin or mucosa or the underlying tissue. The composition can also be formulated for transdermal administration of the therapeutic agent(s) to the systemic circulation, e.g., as a transdermal patch or a microneedle patch.

II. Topical Compositions Comprising a Permeation Enhancer and a Volatile Liquid

in further embodiments, a topical composition comprises an NK-1 antagonist (e.g., serlopitant), a permeation enhancer and a volatile liquid. The composition can optionally contain an additional therapeutic agent. In certain embodiments, the composition contains the NK-1 antagonist (e.g., serlopitant) in free base form.

The permeation enhancer increases the permeability of the skin or mucosa to the therapeutic agent(s). In some embodiments, the permeation enhancer is selected from C₈-C₁₈ alkyl aminobenzoates (e.g., C₈-C₁₈ alkyl p-aminobenzoates), C₈-C₁₈ alkyl dimethylaminobenzoates (e.g., C₅-C₁₈ alkyl p-dimethylaminobenzoates), C₅-C₁₈ alkyl cinnamates, C₈-C₁₈ alkyl methoxy cinnamates (e.g., C₈-C₁₈ alkyl p-methoxycinnamates), and C₈-C₁₈ alkyl salicylates. In certain embodiments, the permeation enhancer is octyl salicylate, octyl p-dimethylaminobenzoate or octyl p-methoxycinnamate, or any combination or all thereof.

The volatile liquid can be any volatile, skin- or mucosa-tolerant solvent. In certain embodiments, the volatile liquid is a C₂-C₅ alcohol or an aqueous solution thereof, such as ethanol or isopropanol or an aqueous solution thereof. An aerosol propellant (e.g., dimethyl ether) can be considered as a volatile liquid. In some embodiments, the volatile liquid functions as a carrier or vehicle of the composition.

The composition can optionally contain a thickening agent. Non-limiting examples of thickening agents include cellulosic thickening agents (e.g., ethyl cellulose, hydroxypropyl cellulose and hydroxypropyl methylcellulose), povidone, polyactylic acids/polyacrylates (e.g., Carbopol® polymers), Sepigel® (polyacrylamide/isoparaffin/laureth-7), and the Gantrez® series of polymethyl vinyl ether/maleic anhydride copolymers (e.g., butyl ester of PMV/MA copolymer Gantrez® A-425).

In some embodiments, the composition contains on a weight basis about 0.5-10%, 0.5-5% or 1-5% of the NK-1 antagonist (e.g., serlopitant), about 1-20%, 1-15% or 1-10% of the permeation enhancer, and about 40-98%, 45-95%, 50-90% or 60-80% of the volatile liquid. In further embodiments, the composition optionally contains on a weight basis about 1-40%, 1-30%, 1-20% or 5-20% water o/land about 0.1-15%, 0.5-10% or 1-5% of a thickening agent.

For purposes of illustration, in certain embodiments a topical spray composition contains about 0.5-5% w/v of the NK-1 antagonist (e.g., serlopitant), about 2-10% w/v of octyl salicylate or octyl p-methyoxycinnamate, and about 95% aqueous ethanol as the carrier. In further embodiments, a topic gel composition comprises about 0.5-5% w/v of the NK-1 antagonist (e.g., serlopitant), about 1-10% w/v of octyl salicylate or octyl p-methyoxycinnamate, about 0.5-5% w/v of a Carbopol® polyacrylic acid, and about 70% aqueous ethanol as the carrier, and optionally about 1-10% w/v of a basic solution (e.g., 0.1 N NaOH). In additional embodiments, a topical lotion composition contains about 0.5-5% w/v of the NK-1 antagonist (e.g., serlopitant), about 1-10% w/v of octyl salicylate or octyl p-methyoxycinnamate, about 1-5% w/v of ethyl cellulose or hydroxypropyl cellulose, and about 90% aqueous ethanol as the carrier.

The composition can further comprise other excipients, such as a compounding agent (e.g., paraffin oil, silicone oil, a vegetable oil, or a fatty ester such as isopropyl myristate), a diluent, a co-solvent (e.g., acetone or a glycol ether such as diethylene glycol monoethyl ether), an emulsifier, a surfactant (e.g., an ethoxylated fatty alcohol, glycerol mono stearate or a phosphate ester), a stabiliser, an antioxidant or a preservative (e.g., a hydroxybenzoate ester), or any combination thereof. For example, a co-solvent or/and a surfactant can be used to maintain the therapeutic agent(s) in solution or suspension at the desired concentration.

The topical composition can have any suitable dosage form, such as a cream, a lotion, a gel, an ointment, a mousse, a spray or aerosol, or any transdermal device (e.g., a patch) that administers a drug by absorption through the skin or mucosa. In some embodiments, the topical composition is applied to the skin or mucosa covering a surface area of about 10-800 cm², 10-400 cm² or 10-200 cm².

III. Topical Compositions Comprising a Permeation Enhancer and Another Excipient

In additional embodiments, a topical composition comprises an NK-1 antagonist (e.g., serlopitant), a permeation enhancer, and at least one of a lipophilic solvent, a formulation base and a thickener. In some embodiments, the composition contains a lipophilic solvent and a formulation base, or the same substance can function as both a lipophilic solvent and a formulation base. In further embodiments, the composition contains a lipophilic solvent, a formulation base and a thickener. The composition can optionally comprise an additional therapeutic agent. In certain embodiments, the composition contains the NK-1 antagonist (e.g., serlopitant) in free base form.

The permeation enhancer increases the permeability of the skin or mucosa to the therapeutic agent(s). Non-limiting examples of permeation enhancers include dimethyl sulfoxide (DMSO), decylmethylsulfoxide, laurocapram, pyrrolidones (e.g., 2-pyrrolidone and N-methyl-2-pyrrolidine), surfactants, alcohols (e.g., oleyl alcohol), polyethylene glycol (e.g., PEG 400), diethylene glycol monoethyl ether, oleic acid, and fatty acid esters (e.g., isopropyl myristate, methyl laurate, glycerol monooleate, and propylene glycol monooleate).

Non-limiting examples of liphophilic solvents include lipophilic alcohols (e.g., hexylene glycol, octyldodecanol, oleyl alcohol and stearyl alcohol), polyethylene glycol (e.g., PEG 100, PEG 300, PEG 400 and PEG 3350), diethylene glycol monoethyl ether, polysorbates (e.g., Tweeng® 20 to 80), Labrasol®, fatty acid esters (e.g., isopropyl myristate and diisopropyl adipate), diethyl sebacate, propylene glycol monocaprylate, propylene glycol laurate, mono- and di-glycerides (e.g., Capmul® MCM), medium-chain triglycerides, caprylic/capric triglyceride, glyceryl monocaprylate, glyceryl mono-oleate, glyceryl mono-linoleate, glycerol oleate/propylene glycol, mineral oil, and vegetable oils.

A liphophilic solvent may also function as a formulation base or carrier. For example, polyethylene glycol (e.g., from PEG 100 to PEG 3500, such as PEG 300, PEG 400 and PEG 3350) can function as a liphophilic solvent and a formulation base.

The composition can also contain a hydrophilic solvent, such as a C₁-C₅ alcohol (e.g., ethanol, isopropanol, glycerol, propylene glycol and 1,2-pentanediol) o/land water.

The composition can contain a thickener to increase the viscosity or/and the physical stability of the composition. Examples of thickeners include without limitation glycerol, stearyl alcohol, and polymers (e.g polydimethylsiloxane [dimethicone] and Carbopol® polymers).

In some embodiments, the composition further contains an antioxidant. Non-limiting examples of antioxidants include butylated hydroxyanisole (BHA), butylated hydroxytoluene (BHT), tocopherols (e.g., Vitamin E and esters thereof), flavinoids, glutathione, ascorbic acid and esters thereof, DMSO, and chelating agents (e.g., EDTA and citric acid).

In certain embodiments, the topical composition comprises on a w/w basis about 0.5-10% or 1-5% of the NK-1 antagonist (e.g., serlopitant), about 2-30% or 5-20% of a permeation enhancer, about 20-80% or 30-70% of a lipophilic solvent that may also function as a formulation base, about 0.1-10% or 1-7.5% of a thickener, and about 0.01-2% or 0.054% of an antioxidant. As a non-limiting example, a topical composition can contain the NK-1 antagonist (e.g., serlopitant), PEG 400 or/and PEG 3350 as lipophilic solvent(s) and formulation base(s), diethylene glycol monoethyl ether, oleyl alcohol or/and isopropyl myristate as permeation enhancer(s), stearyl alcohol as a thickener, and BHT as an antioxidant.

The topical composition can have any suitable dosage form, such as a cream, a lotion, a gel, an ointment, a jelly, a paste, or any transdermal device (e.g., a patch) that administers a drug by absorption through the skin or mucosa.

IV. Topical Compositions Comprising a Permeation Enhancer and an Adhesive

In other embodiments, a topical composition comprises an NK-1 antagonist (e.g., serlopitant), a permeation enhancer and an adhesive. The composition can optionally contain an additional therapeutic agent. In certain embodiments, the composition contains the NK-1 antagonist (e.g., serlopitant) in free base form.

The permeation enhancer increases the permeability of the skin or mucosa to the therapeutic agent(s). The permeation enhancer can be, e.g., a fatty acid ester having a fatty acyl chain length of C₈-C₂₀ or C₁₂-C₁₈ and a C₁-C₆ or C₂-C₄ alcohol component (e.g, isopropanol). In certain embodiments, the permeation enhancer is isopropyl myristate or isopropyl palmitate. In some embodiments, the permeation enhancer is in an amount of about 0.1-20%, 0.5-15%, 1-15%, 2-12% or 4-10% by weight of the composition or the skin-contacting layer of a transdermal patch.

The adhesive maintains contact of the topical composition to the skin or mucosa. Non-limiting examples of adhesives include acrylics/acrylates (e.g polyacrylates, including polyalkyl acrylates and Duro-Tak® polyacrylates), polyvinyl acetate, ethylenevinylacetate copolymers, polysiloxanes, polyurethanes, plasticized polyether block amide copolymers, natural and synthetic rubbers, plasticized styrene-butadiene rubber block copolymers (e.g., Duro-Tak® 87-6173), and mixtures thereof.

The topical composition can comprise one or more additional excipients. The additional excipient(s) can be, e.g., a diluent, an emollient, a plasticizer, or an agent that reduces irritation to the skin or mucosa, or any combination thereof.

In certain embodiments, the topical composition prior to application to the skin or mucosa is substantially free of water, tetraglycol (glycofurol) or/and a hydrophilic organic solvent (e.g., a C₁-C₅ alcohol).

The composition can administer the therapeutic agent(s) transdermally (including percutaneously and transmucosally) through a body surface or membrane such as intact unbroken skin or intact unbroken mucosal tissue into the systemic circulation.

In some embodiments, the topical composition is in the form of a transdermal patch for application to the skin or mucosa. In certain embodiments, the patch has a skin- or mucosa-contacting layer (“skin-contacting layer” for simplicity) laminated or otherwise attached to a support layer. The skin-contacting layer can be covered by a removable release liner before use to protect the skin-contacting surface and to keep it clean until it is applied to the skin or mucosa.

The support layer of the patch acts as a support for the skin-contacting layer and as a barrier that prevents loss of the therapeutic agent(s) in the skin-contacting layer to the environment. The material of the support layer is compatible with the therapeutic agent(s), the permeation enhancer and the adhesive, and is minimally permeable to the components of the patch. The support layer can be opaque to protect the components of the patch from degradation via exposure to ultraviolet light. The support layer is also capable of binding to and supporting the adhesive layer, yet is sufficiently pliable to accommodate the movements of the subject using the patch. The material of the support layer can be, e.g., a metal foil, a metalized polyfoil, or a composite foil or film containing a polymer (e.g., a polyester [such as polyester terephthalate] or aluminized polyester, polyethylene, polypropylene, polytetrafluoroethylene, a polyethylene methyl methacrylate block copolymer, a polyether block amide copolymer, a polyurethane, polyvinylidene chloride, nylon, a silicone elastomer, rubber-based polyisobutylene, styrene, or a styrene-butadiene or styrene-isoprene copolymer). The release liner can be made of the same material as the support layer, or can be a film coated with an appropriate release surface.

Combination Therapies with an NK-1 Antagonist and Other Anti-Pruritic Agents

One or more additional antipruritic agents can optionally be used in combination with an NK-1 antagonist (e.g., serlopitant) to treat acute or chronic pruritus associated with a condition described herein. The NK-1 antagonist may synergize or enhance the activity of the one or more additional antipruritic agents.

Examples of antipruritic agents include without limitation:
opioid receptor (e.g., mu-opioid receptor) antagonists, including but not limited to alvimopan, axelopran, bevenopran, butorphanol (a mu antagonist and kappa agonist), cyprodime, eptazocine, levallorphan (lorfan or naloxiphan), methylnaltrexone, naldemedine, nalmefene, nalbuphine (a mu antagonist and kappa agonist), nalodeine, nalorphine (lethidrone or nalline), naloxegol, naloxone, naloxol, naltrexone (e.g., naltrexone 1% cream), 6β-naltrexol, samidorphan, SK-1405, and analogs, derivatives and salts thereof;
opioid receptor agonists, including but not limited to selective kappa-opioid receptor agonists such as asimadoline, bremazocine, difelikefalin (CR845), dynorphin, enadoline, ketazocine, nalfurafine (TRK-820), salvinorin A, 2-methoxymethyl salvinorin B, 2-ethoxymethyl salvinorin B, 2-fluoroethoxymethyl salvinorin B, spiradoline, tifluadom, BRL-52537, FE 200665, GR-89696, HZ-2, ICI-199,441, ICI-204,448, LPK-26, SA-14867, U-50488, U-69,593, 2-phenylbenzothiazoline-type compounds, and analogs, derivatives and salts thereof;
agonists of cannabinoid receptors (e.g., CB₁ and CB₂), including but not limited to CB₂ agonists anandamide [N-arachidonoylethanolamine], 2-arachidonoylglycerol, virodhamine [O-arachidonoylethanolamine], palmitoylethanolamide [PEA, N-palmitoylethanolamine], AM-1241, GW-405833, HU-308, JWH-015, JWH-133, L-759633, L-759656 and S-777469), and analogs, derivatives and salts thereof;
fatty acid amide hydrolase (FAAH) inhibitors, including but not limited to ARN2508, BMS-469908, CAY-10402, JNJ-245, JNJ-1661010, SNJ-28833155, JNJ-40413269, JNJ-42119779, JNJ-42165279, LY-2183240, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, and analogs, derivatives and salts thereof;
antagonists of transient receptor potential cation channels, including but not limited to transient receptor potential ankyrin A1 (TRPA1) antagonists {e.g., camphor, isopentenyl pyrophosphate, A967079, GRC-17536, (4R)-1,2,3,4-tetrahydro-4-[3-(3-methoxypropoxy)phenyl]-2-thioxo-5H-indeno[1,2-d]pyrimidin-5-one, 2-amino-4-arylthiazole compounds disclosed in WO 2012/085662 A1, and specialized pro-resolving mediators (SPMs) (e.g., metabolites of polyunsaturated fatty acids [PUFAs])}, transient receptor potential vanilloid (TRPV) antagonists (e.g., TRPV1 antagonists [e.g., capsazepine, iodo-resiniferatoxin, AMG-517, GRC-6211, NGD-8243, SB-705498 and SPMs {e.g., PUFA metabolites}] and TRPV3 antagonists [e.g., SPMs {e.g., PUFA metabolites}]), and analogs, derivatives and salts thereof;
TRPV1 agonists that cause decrease in TRPV1 activity (desensitization) upon prolonged exposure of TRPV1 to the stimuli, including but not limited to capsaicin, camphor, carvacrol, menthol, methyl salicylate, resiniferatoxin, tinyatoxin, and analogs, derivatives and salts thereof;
antagonists of Mas-related G-protein coupled receptors (MRGPRs), including but not limited to MRGPRX1 antagonists (e.g., 2-diphenylmethyl-3-substituted azabicyclo-octanes disclosed in P. Kunapuli et al., Anal. Biochem., 351:50-61 [2006]), MRGPRX2 antagonists (e.g., Gln-Trp-Phe, Gln-Trp-Phe-NH₂, Ac-Gln-Trp-Phe-NH₂, Gln-D-Trp(formyl)-Phe benzyl ester, Boc-Gln-D-Tip(formyl)-Phe benzyl ester), and analogs, derivatives and salts thereof;
antagonists of protease-activated receptors (PARs) and inhibitors of activating proteases, including but not limited to PAR1 antagonists (e.g., SCH-530,348), PAR2 antagonists {e.g., AY-117, ENMD-1068, ENMD-106836, GB-83, tetracyclines (e.g., doxycycline, minocycline and tetracycline), FSLLRY-NH₂ (PAR-3888-PD, Ac-FSLLRY-NH₂ and anti-PAR2 antibodies (e.g., SAM-11 [SC-13504], P2pa1-21 and P2pa1-2135}, PAR4 antagonists {e.g, ethanol, YD-3, statins atorvastatin, cerivastatin, fluvastatin, lovastatin, pitavastatin, pravastatin, rosuvastatin and simvastatin), pepducin P4 pal-10, pepducin P4 pal-15, trans-cinnamoyl-APGKF-NH₂, trans-cinnamoyl-YPGKF-NH₂, and anti-PAR4 antibodies (e.g., C-19 and SC-1249)}, inhibitors of serine proteases {e.g., benzamidine hydrochloride, 4-iodo-1-benzothiophene-2-carboximidamide hydrochloride (inhibits trypsin and tryptase), inhibitors of kallikreins (e.g., camostat, nafamostat, gabexate, ecallantide and α₁-inhibitor), trypsin inhibitors tosyllysine chloromethyl ketone [TLCK] hydrochloride, α₁-antitrypsin, aprotinin, ovomucin and soybean trypsin inhibitor), and tryptase inhibitors (e.g., camostat, nafamostat, gabexate, AMG-126737 and APC-366)}, inhibitors of cysteine proteases {e.g., E-64 (non-specific inhibitor), JNJ-10329670, RWJ-445380, cystatin C, leupeptin, stefin A, stefin B, testican-1, chloroquine, fluoromethyl ketone, naphthalene endoperoxide (inhibits cathepsin B, L and S), CA-074 (inhibits cathepsin B), odanacatib (MK-0822, inhibits cathepsin K), CLIK-148 and CLIK-195 (inhibit cathepsin L), and CLIK-60 and E-6438 (inhibit cathepsin S)}, and analogs, derivatives, fragments and salts thereof;
antagonists of endothelin receptors, including but not limited to selective endothelin A receptor (ETAR) antagonists {e.g., ambrisentan, atrasentan, sitaxentan, zibotentan, BQ-123, 4-amino-N-(3,4-dimethylisoxazol-5-yl)benzenesulfonamide; (2R)-2-[[(2R)-2-[[(2 S)-2-(azepane-1-carbonylamino)-4-methylpentanoyl]amino]-3-(1-formylindol-3-yl)propanoyl]amino]-3-(1H-indol-3-yl)propanoic acid; 3-benzodioxol-5-yl)-1-[2-(dibutylamino)-2-oxoethyl]-2-(4-methoxyphenyl)pyrrolidine-3-carboxylic acid; (2R,3R,4S)-4-(1,3-benzodioxol-5-yl)-1-[2-(dibutylamino)-2-oxoethyl]-2-(4-methoxyphenyl)pyrrolidine-3-carboxylic acid; (2R,3R,4S)-4-(1,3-benzodioxol-5-yl)-1[2-(dibutylamino)-2-oxoethyl]-2-(2-methoxyphenyl)pyrrolidine-3-carboxylic acid; 3-(1,3-benzodioxol-5-yl)-5-hydroxy-5-(4-methoxyphenyl)-4-[(3,4,5-trimethoxyphenyl)methyl]furan-2-one; 2-(1,3-benzodioxol-5-yl)-4-(4-methoxyphenyl)-4-oxo-3-[(3,4,5-trimethoxyphenyl)methyl]but-2-enoate; 5-(4-bromophenyl)-6-[2-(5-bromopyrimidin-2-yl)oxyethoxy]-N-(propylsulfamoyl)pyrimidin-4-amine; 4-tert-butyl-N-[6-(2-hydroxyethoxy)-5-(2-methoxyphenoxy)-2-(pyrimidin-2-yl)pyrimidin-4-yl]benzenesulfonamide; [(7R)-5-chloro-3-[(1E,3E,5S)-3,5-dimethylhepta-1,3-dienyl]-7-methyl-6,8-dioxoisochromen-7-yl]acetate; N-(4-chloro-3-methyl-1,2-oxazol-5-yl)-2-[2-(6-methyl-2H-1,3-benzodioxol-5-yl)acetyl]thiophene-3-sulfonamide; (2S)-2-(4,6-dimethoxypyrimidin-2-yl)oxy-3-methoxy-3,3-diphenylpropanoic acid; (2S)-2-[(4,6-dimethylpyrimidin-2-yl)oxyl-3-methoxy-3,3-diphenylpropanoic acid; N-[6-(2-hydroxyethoxy)-5-(2-methoxyphenoxy)-2-[2-(2H-tetrazol-5-yl)pyridin-4-yl]pyrimidin-4-yl]-5-methylpyridine-2-sulfonamide; N-[6-(2-hydroxyethoxy)-5-(2-methoxyphenoxy)-2-[2-(2H-tetrazol-5-yl)pyridin-4-yl]pyrimidin-4-yl]-5-propan-2-ylpyridine-2-sulfonamide; 6-(2-hydroxy-ethoxy)-5-(2-methoxyphenoxy)-2-[2-(1,2,3-triaza-4-azanidacyclopenta-2,5-dien-5-yl)pyridin-4-yl]pyrimidin-4-yl]-(5-methylpyridin-2-yl)sulfonylazanide; 2-[(3R,6R,9S,12R,15S)-6-(1H-indol-3-ylmethyl)-9-(2-methylpropyl)-2,5,8,11,14-pentaoxo-12-propan-2-yl-1,4,7,10,13-pentazabicyclo[13.3.0]octadecan-3-yl]acetic acid; N-[6-methoxy-5-(2-methoxyphenoxy)-2-pyridin-4-ylpyrimidin-4-yl]-5-methylpyridisulfonamide; N-(3-methoxy-5-methylpyrazin-2-yl)-2-[4-(1,3,4-oxadiazol-2-yl)phenyl]pyridine-3-sulfonamide; and N-[5-(2-methoxyphenoxy)-2-pyridin-4-yl-6-(trideuteriomethoxy)pyrimidine-4-yl]-5-methylpyridine-2-sulfonamide}, selective endothelin B receptor (ETBR) antagonists (e.g., A-192621 and BQ-788), dual ETAR/ETBR antagonists (e.g., bosentan, macitentan and tezosentan), and analogs, derivatives and salts thereof;
inhibitors of Toll-like receptors (TLRs), including, but not limited to TIR7/non-TLR9 inhibitors (e.g., ODN 2087, ODN 20958 and ODN 20959), dual TLR7/TLR9 inhibitors (e.g., chloroquine, hydroxychloroquine, quinacrine, AT791, DV056, E6446, IMO-3100, IMO-8400 and ODN 2088), and analogs, derivatives, fragments and salts thereof;
inhibitors of mitogen-activated protein (MAP) kinases, including but not limited to p38 MAP kinase inhibitors {e.g., BMS-582949, CPSI-2364, 4-(4-fluorophenyl)-2-(4-hydroxyphenyl)-5-(4-pyridyl)-1H-imidazole, trans-4-[4-(4-fluorophenyl)-5-(2-methoxy-4-pyrimidinyl)-1H-imidazole-1-yl-]cyclohexanol, and 4-(4-fluorophenyl)-2-(4-methylsulfinylphenyl)-5-(4-pyridyl)-1H-imidazole}, and analogs, derivatives and salts thereof;
inhibitors of mitogen-activated protein kinase kinases (MEKs), including but not limited to MEK 1 inhibitors {e.g., N-[3-[5-(2-aminopyrimidin-4-yl)-2-tert-butyl-1,3-thiazol-4-yl]-2-fluorophenyl]-2,6-difluorobenzenesulfonamide; N-[3-[5-(2-aminopyrimidin-4-yl)-2-tert-butyl-1,3-thiazol-4-yl]-2-fluorophenyl]-2,6-difluorobenzenesulfonamide, methanesulfonic acid; 6-(4-bromo-2-chloroanilino)-7-fluoro-N-(2-hydroxyethoxy)-3-methylbenzimidazole-5-carboxamide; 5-bromo-N-(2,3-dihydroxypropoxy)-3,4-difluoro-2-(2-fluoro-4-iodoanilino)benzamide; 6-(4-bromo-2-fluoroanilino)-7-fluoro-N-(2-hydroxyethoxy)-3-methylbenzimidazole-5-carboxamide; 2-[4-[(2-butyl-4-oxo-1,3-diazaspiro[4.4]non-1-en-3-yl)methyl]-2-(ethoxymethyl)phenyl]-N-(3,4-dimethyl-1,2-oxazol-5-yl)benzenesulfonamide; 2-[4-[(2-butyl-4-oxo-1,3-diazaspiro[4.4]non-1-en-3-yl)methyl]-2-(ethoxymethyl)phenyl]-N-(4,5-dimethyl-1,2-oxazol-3-yl)benzenesulfonamide; 2-[4-[(2-butyl-4-oxo-1,3-diazaspiro [4.4]non-1-en-3-yl)methyl]-2-propylphenyl]-N-(4,5-dimethyl-1,2-oxazol-3-yl)benzenesulfonamide; 2-(2-chloro-4-iodoanilino)-N-(cyclopropylmethoxy)-3,4-difluorobenzamide; N-[3-[3-cyclopropyl-5-(2-fluoro-4-iodoanilino)-6,8-dimethyl-2,4,7-trioxopyrido[4,3-d]pyrimidin-1-yl]phenyl]acetamide; 3,4-difluoro-2-(2-fluoro-4-iodoanilino)-N-(2-hydroxyethoxy)-5-[(3-oxooxazinan-2-yl)methyl]benzamide; N-[3,4-difluoro-2-(2-fluoro-4-iodoanilino)-6-methoxyphenyl]-[(2S)-2,3-dihydroxypropyl]cyclopropane-1-sulfonamide; [3,4-difluoro-2-(2-fluoro-4-iodoanilino)phenyl]-[3-hydroxy-3-[(2S)-piperidin-2-yl]azetidin-1-yl]methanone; N-[(2R)-2,3-dihydroxypropoxy]-3,4-difluoro-2-(2-fluoro-4-iodoanilino)benzamide; (2S,3S)-2-[(4R)-4-[4-[(2R)-2,3-dihydroxypropoxy]phenyl]-2,5-dioxoimidazolidin-1-yl]-N-(2-fluoro-4-iodophenyl)-3-phenylbutanamide; 3-[(2R)-2,3-dihydroxypropyl]-6-fluoro-5-(2-fluoro-4-iodoanilino)-8-methylpyrido[2,3-(1]pyrimidine-4,7-dione; N-[(2S)-2,3-dihydroxypropyl]-3-(2-fluoro-4-iodoanilino)pyridine-4-carboxamide, and 2-(2-fluoro-4-iodoanilino)-N-(2-hydroxyethoxy)-1,5-dimethyl-6-oxopyridine-3-carboxamide}, and analogs, derivatives and salts thereof;
inhibitors of calcitonin gene-related peptide (CGRP) or receptor therefor or the production thereof, including but not limited to CORP receptor antagonists (e.g., olcegepant, telcagepant, ubrogepant, eptinezumab [ALD-403], AMG-334, LY-2951742 and TEV-48125), and analogs, derivatives, fragments and salts thereof;
inhibitors of gastrin-releasing peptide (GRP) or the receptor therefor (GRPR, aka bombesin receptor 2 [BBR2]) or the production thereof, including but not limited to CRPR antagonists (e.g.; RC-3095), and analogs, derivatives and salts thereof;
inhibitors of nerve growth factor (NGF) or receptors therefor tropomyosin kinase receptor A [TrkA]) or the production thereof, including but not limited to NGF inhibitors (e.g., fulranumab and tanezumab), NGF receptor inhibitors (e.g., TrkA inhibitors such as A0879, CT327 and K252a), and analogs, derivatives, fragments and salts thereof;
inhibitors of neurotensin or receptors therefor (e.g., neurotensin receptor 1 [NTSR1], NTSR2 and so 1) or the production thereof, including but not limited to selective NTSR1 antagonists (e.g., SR-48,692), selective NTSR2 antagonists (e.g, levocabastine), unselective receptor antagonists (e.g., SR-142,948), and analogs, derivatives and salts thereof;
inhibitors of somatostatin or receptors therefor (e.g., somatostatin receptors [SSTRs] 1 to 5) or the production thereof, including but not limited to selective SSTR2 antagonists (e.g., CYN 154806), selective SSTRS antagonists (e.g., BIM 23056), unselective SSTR antagonists (e.g., cyclosomatostatin), and analogs, derivatives, fragments and salts thereof;
inhibitors of vasoactive intestinal peptide (VIP) or receptors therefor (e.g., VIPR1 and VIPR2) or the production thereof, including but not limited to VIP receptor antagonists {e.g., PG 97-269, ViPhyb, VIP(6-28)-NH₂, [p-Cl-D-Phe⁶, Leu¹⁷]VIP-NH₂, [Ac-His¹, D-Phe², Lys¹⁵, Arg¹⁶]VIP(3-7)GRF(8-27)-NH₂, and [Ac-Tyr¹, D-Phe²]GRF(1-29)-NH₂}, and analogs, derivatives, fragments and salts thereof;
inhibitors of bradykinin or receptors therefor (e.g., B1 and B2) or the production thereof, including but not limited to bradykinin inhibitors (e.g., aloe, bromelain and polyphenols), bradykinin receptor B2 antagonists (e.g., icatibant and FR-173657), inhibitors of kallikreins (e.g., ecallantide, camostat, nafamostat, gabexate and C1-inhibitor), and analogs, derivatives and salts thereof;
inhibitors of corticotropin-releasing hormone (CRH, aka corticoliberin) or receptors therefor (e.g., CRHR1 and CRHR2) or the production thereof, including but not limited to CRHR1 antagonists (e.g., antalarmin, pexacerfont, CP-154,526 LWH-234, NBI-27914 and R-121,919), CRHR2 antagonists (e.g., astressin-B), and analogs, derivatives and salts thereof;
antihistamines, including but not limited to antihistamines that inhibit action at the histamine H₁ receptor (e.g., acrivastine, antazoline, astemizole, azatadine, azelastine, bepotasiine, bilastine, bromodiphenhydramine, brompheniramine, buclizine, carbinoxamine, cetirizine, chlorcyclizine, chlorodiphenhydramine, chlorpheniramine, chlorpromazine, chloropyramine, cidoxepin, clemastine, cyclizine, cyproheptadine, desloratadine, dexbrompheniramine, dexchlorpheniramine, dimenhydrinate, dimetindene, diphenhydramine, doxepin, doxylamine, ebastine, embramine, esmirtazapine [(S)-(+)-enantiomer of mirtazapine], fexofenadine, hydroxyzine, ketotifen, levocabastine, levocetirizine, loratadine, meclozine mepyramine, mirtazapine, mizolastine, olopatadine, orphenadrine, phenindamine, pheniramine, phenyltoloxamine, promethazine, pyrilamine, quetiapine, quifenadine, rupatadine, terfenadine, trimeprazine tripelennamine and triprolidine), antihistamines that inhibit action at the histamine H₃ receptor (e.g., betahistine, burimamide, ciproxifan, clobenpropit, conessine, failproxifan, impentamine, iodophenpropit, irdabisant, pitolisant, thioperamide, A-349,821, ABT-239 and VUF-568), antihistamines that inhibit action at the histamine H₄ receptor (e.g., clobenpropit, thioperamide, A943931, A987306, JNJ-7777120, VUF-6002 and ZPL-389), and analogs, derivatives and salts thereof;
inhibitors of phospholipase A2 (e.g., secreted and cytosolic PLA2), including but not limited to arachidonyl trifluoromethyl ketone, bromoenol lactone, chloroquine, cytidine 5-diphosphoamines, darapladib, quinacrine, vitamin E, RO-061606, ZPL-521, lipocortins (annexins), and analogs, derivatives, fragments and salts thereof;
inhibitors of pro-inflammatory prostaglandins (e.g, prostaglandin E2) or receptors therefor or the production thereof, including but not limited to non-steroidal anti-inflammatory drugs (NSAIDs) (e.g., non-selective COX-1/COX-2 inhibitors such as aspirin and selective COX-2 inhibitors such as coxibs), glucocorticoids, cyclopentenone prostaglandins (e.g., prostaglandin J2 [PGJ2], Δ12-PGJ2 and 15-deoxy-Δ12,14-PGJ2), and analogs, derivatives and salts thereof, inhibitors of leukotrienes or receptors therefor or the production thereof, including but not limited to leukotriene receptor antagonists (e.g., cinalukast, gemilukast, iralukast, montelukast, pranlukast, tomelukast, verlukast, zafirlukast, CP-199330, HAMI-3379, ICI-198615 and MK-571), 5-lipoxygenase inhibitors (e.g., baicalein, caffeic acid, curcumin, hyperforin, meclofenamic acid, meclofenamate sodium, zileuton and MK-886), and analogs, derivatives and salts thereof;
mast cell stabilizers, including but not limited to cromoglicic acid (cromolyn), ketotifen, methylxanthines, nedocromil, olopatadine, omalizumab, pemirolast, quercetin. β₂-adrenoreceptor agonists {including short-acting β₂-adrenergic agonists (e.g., bitolterol, fenoterol, isoprenaline [isoproterenol], levosalbutamol [levalbuterol], orciprenaline [metaproterenol], pirbuterol, procaterol, ritodrine, salbutamol [albuterol] and terbutaline), long-acting β₂-adrenergic agonists arformoterol, bambuterol, clenbuterol, formoterol and salmeterol), and ultralong-acting β₂-adrenergic agonists (e.g., carmoterol, indacaterol, milveterol, olodaterol and vilanterol)}, and analogs, derivatives and salts thereof;
Janus kinase (JAX) inhibitors, including, but not limited to JAK1 inhibitors (e.g., GLPG0634 and GSK2586184). JAK2 inhibitors (e.g., lestaurtinib, pacritinib, CYT387 and TG101348), JAK3 inhibitors (e.g., ASP-015K, 8348 and VX-509), dual JAK1/JAK2 inhibitors (e.g., baricitinib and ruxolitinib), dual JAK1/JAK3 inhibitors (e.g., tofacitinib), and analogs, derivatives and salts thereof;
immunomodulators, including but not limited to imides (e.g., thalidomide, lenalidomide, pomalidomide and apremilast), xanthine derivatives (e.g., lisofylline, pentoxifylline and propentofylline), and analogs, derivatives and salts thereof;
immunosuppressants, including but not limited to glucocorticoids, antimetabolites (e.g., hydroxyurea [hydroxycarbamide], antifolates [e.g., methotrexate], and purine analogs [e.g., azathioprine, mercaptopurine and thioguanine]), calcineurin inhibitors (e.g, ciclosporin [cyclosporine A], pimecrolimus and tacrolimus), inosine-5′-monophosphate dehydrogenase (IMPDH) inhibitors (e.g., mycophenolic acid and derivatives thereof [e.g., mycophenolate sodium and mycophenolate mofetil]), mechanistic/mammalian target of rapamycin (mTOR) inhibitors (e.g., rapamycin [sirolimus], deforolimus [ridaforolimus], everolimus, temsirolimus, umirolimus [biolimus A9], zotarolimus and RTP-801), modulators of sphingosine-1-phosphate receptors (e.g., SIPR1) (e.g., fingolimod), serine C-palmitoyltransferase inhibitors (e.g., myriocin), and analogs, derivatives and salts thereof;
corticosteroids/glucocorticoids, including but not limited to hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), carbonates (e.g., prednicarbate), and analogs, derivatives and salts thereof;
inhibitors of pro-inflammatory cytokines or receptors therefor, including but not limited to inhibitors of (e.g., antibodies to) tumor necrosis factor-alpha (TNF-α) (e.g, adalimumab, certolizumab pegol, golimumab, infliximab, etanercept, bupropion and ART-621), inhibitors of (e.g., antibodies to) pro-inflammatory interferons (e.g., interferon-alpha [IFN-α]) or receptors therefor, inhibitors of (e.g., antibodies to) pro-inflammatory interleukins or receptors therefor (e.g., IL-1 [e.g., IL-1α and IL-1β] or IL-1R [e.g., EBI-005 {isunakinra}], IL-2 or IL-2R [e.g., basiliximab and daclizumab], IL-4 or IL-4R [e.g., dupilumab], IL-5 [e.g., mepolizumab] or IL-5R, IL-6 [e.g., clazakizumab, elsilimomab, olokizumab, siltuximab and sirukumab] or IL-6R [e.g., sarilumab and tocilizumab], IL-8 or IL-8R, IL-12 [e.g., briakinumab and ustekinumab] or IL-12R, IL-13 or IL-13R, IL-15 or IL-15R, IL-17 [e.g., ixekizumab and secukinumab] or IL-17R [e.g., brodalumab], IL-18 or IL-18R, IL-20 [e.g., the antibody 7E] or IL-20R, IL-22 [e.g., fezakinumab] or IL-22R, IL-23 [e.g., briakinumab, guselkumab, risankizumab, tildrakizumab SCH-9002221, ustekinumab and BI-655066] or IL-23R, IL-31 or IL-31R [e.g., anti-IL-31 receptor A antibodies such as nemolizumab], IL-33 or IL-33R, and IL-36 or IL-36R), and analogs, derivatives, fragments and salts thereof;
inhibitors of the production of pro-inflammatory cytokines or receptors therefor, including but not limited to inhibitors of the production of TNF-α (e.g., myxoma virus M013 protein, Yersinia YopM, protein, glucocorticoids, immunomodulatory imides, PDE4 inhibitors, p38 MAP kinase inhibitors, inhibitors of TLRs such as TLR7 and TLR9, scrim protease inhibitors [e.g., gabexate and nafamostat], and prostacyclin, carbacyclin and analogs and derivatives thereof [e.g., beraprost, cicaprost, ciprosten, eptaloprost, iloprost and treprostinil]), IFN-α (e.g., alefacept and inhibitors of TLRs such as TLR7 and TLR9), IL-1 (e.g., IL-1α, and IL-1β) (e.g., M013 protein, YopM protein, nafamostat, prostacyclin, glucocorticoids, TNF-α inhibitors, inhibitors of TLRs such as TLR7 and TLR9, and PAR1 antagonists), IL-2 (e.g., glucocorticoids, calcineurin inhibitors and PDE4 inhibitors), IL-4 (e.g., glucocorticoids and serine protease inhibitors [e.g., gabexate and nafamostat]), IL-5 (e.g., glucocorticoids), IL-6 M013 protein, nafamostat, prostacyclin, tranilast, glucocorticoids, immunomodulatory imides, TNF-α inhibitors, and inhibitors of TLRs such as TLR7 and TLR9), IL-8 alefacept, glucocorticoids and PAR2 antagonists [e.g., tetracyclines]), IL-12 (e.g., apilimod, YopM protein, PDE4 inhibitors, and inhibitors of TLRs such as TLR7 and TLR9), IL-15 (e.g., YopM protein), IL-17 (e.g., protein kinase C [PKC] inhibitors such as sotrastaurin), IL-18 (e.g., MOD protein and YopM protein), and IL-23 (e.g., apilimod, alefacept and PDE4 inhibitors), and analogs, derivatives, fragments and salts thereof;
other kinds of anti-inflammatory agents, including but not limited to inhibitors of pro-inflammatory transcription factors e.g., inhibitors of NE-κB [e.g., nafamostat, M013 protein, penetranin, (−)-DHMEQ, IT-603, IT-901 and PBS-1086] and inhibitors of STAT [signal transducer and activator of transcription] proteins [e.g., JAK1, JAK2 and JAK3 inhibitors]), antagonists of the prostaglandin D₂ receptor (DP₁) or/and the chemoattractant receptor homologous molecule expressed on TH₂ cells (CRTH2) (e.g., TS-022), phosphodiesterase (PDE) inhibitors (e.g., PDE4 inhibitors such as apremilast, cilomilast, ibudilast, piclamilast, roflumilast, crisaborole, diazepam, luteolin, mesembrenone, rolipram, AN2728 and E6005), IgE inhibitors (e.g., anti-IgE antibodies such as omalizumab), myeloperoxidase inhibitors (e.g., dapsone), specialized pro-resolving mediators (SPMs) (e.g., metabolites of polyunsaturated fatty acids such as lipoxins, resolvins [including resolvins derived from 5Z,8Z,11Z,14Z,17Z-eicosapentaenoic acid {EPA}, resolvins derived from 4Z,7Z,10Z,13Z,16Z,19Z-docosahexaenoic acid {DHA}, and resolvins derived from 7Z,10Z,13Z,16Z,19Z-docosahexaenoic acid {n-3 DPA}], protectins/neuroprotectins [including DHA-derived protectins/neuroprotectins and n-3 DPA-derived protectins/neuroprotectins], maresins [including DHA-derived maresins and n-3 DPA-derived maresins], n-3 DPA metabolites, n-6 DPA {4Z,7Z,10Z,13Z,16Z-docosapentaenoic acid} metabolites, oxo-DHA metabolites, oxo-DPA metabolites, docosahexaenoyl ethanolamide metabolites, cyclopentenone prostaglandins [e.g., Δ12-PGJ2 and 15-deoxy-Δ12,14-PGJ2], and cyclopentenone isoprostanes [e.g., 5,6-epoxyisoprostane A2 and 5,6-epoxyisoprostane E2]), disease-modifying antirheumatic drugs (DMARDs, e.g., sulfasalazine and mesalazine [5-aminosalicylic acid]), anti-allergic agents (e.g., antihistamines, inhibitors of leukotrienes or receptors therefor or the production thereof, mast cell stabilizers, glucocorticoids, epinephrine [adrenaline] and tranilast), ultraviolet radiation (e.g., ultraviolet A and B), and analogs, derivatives, fragments and salts thereof;
antagonists of serotonin receptors, including but not limited to 5-HT₂ antagonists (e.g., clozapine, cyproheptadine ketanserin, pizotifen [pizotyline] and quetiapine), 5-HT₃ antagonists (e.g., alosetron, bemesetron, cilansetron, dolasetron, granisetron, ondansetron, palonosetron, ricasetron, tropanserin, tropisetron, zatosetron, mirtazapine, esmirtazapine and substances present in ginger [e.g., galanolactone, gingerols and shogaols]), and analogs, derivatives and salts thereof;
antagonists of muscarinic acetylcholine receptors (e.g., M1 to M5), including but not limited to aclidinium, atropine, benzatropine, biperiden, chlorpheniramine, cyclopentolate, darifenacin, dicyclomine, dimenhydrinate, diphenhydramine, doxepin, doxylamine, flavoxate, glycopyrrolate, hyoscyamine, ipratropium, orphenadrine, oxitropium, oxybutynin, pirenzepine, procyclidine, scopolamine (hyoscine), solifenacin, tolterodine, tiotropium, trihexyphenidyl, tropicamide, tricyclic antidepressants, and analogs, derivatives and salts thereof;
antidepressants, including but not limited to tricyclic antidepressants (e.g., amitriptyline, amitriptylinoxide, amoxapine, dosulepin [dothiepin], doxepin, cidoxepin and melitracen), tetracyclic antidepressants (e.g., amoxapine, maprotiline, mazindol, mianserin, mirtazapine, esmirtazapine and setiptiline), selective serotonin reuptake inhibitors (SSRIs, e.g., citalopram, dapoxetine, escitalopram, fluoxetine, fluvoxamine, paroxetine and sertraline), serotonin-norepinephrine reuptake inhibitors (SNRIs, e.g., bicifadine, doxepin, cidoxepin, dapoxetine, milnacipran, levomilnacipran, sibutramine, venlafaxine, desvenlafaxine and SEP-227162), inhibitors of monoamine oxidases (MAOs) (e.g., selective MAO-A inhibitors [e.g., bifemelane, moclobemide, pirlindole [pirazidol] and toloxatone], selective MAO-B inhibitors [e.g., rasagiline and selegiline], and non-selective MAO-A/MAO-B inhibitors [e.g., hydracarbazine, isocarboxazid, nialamide, phenelzine and tranylcypromine]), and analogs, derivatives and salts thereof;
anticonvulsants, including but not limited to carbamazepine, gabapentin, pregabalin, topiramate, valproic acid and salts thereof (e.g., sodium valproate), and analogs, derivatives and salts thereof;
counterirritants and cooling agents, including but not limited to capsaicin, camphor, eucalyptol, icilin, isopulegol, mint oil (e.g., Japanese mint [Mentha arvensis] oil, peppermint oil and spearmint oil), menthol (e.g., menthol 1-3% cream), meteoric, menthone glycerol ketal, menthyl lactate, menthyl succinate, methyl salicylate, phenol (e.g., in calamine cream and lotion), trimethylcyclohexanol, WS-23, local anesthetics, and analogs, derivatives and salts thereof;
local anesthetics, including but not limited to amides (e.g., articaine, bupivacaine, cinchocaine [dibucaine], etidocaine, levobupivacaine, lidocaine [e.g., lidocaine 2.5-5% cream], prilocaine [e.g., prilocaine 2.5% cream], EMLA [lidocaine 2.5%/prilocaine 2.5% cream], mepivacaine, ropivacaine and trimecaine), esters (e.g., benzocaine, chloroprocaine, cocaine, cyclomethycaine, dimethocaine [larocaine], piperocaine, procaine [novocaine], proparacaine, propoxycaine, stovaine and tetracaine [amethocaine]), ethers (e.g., polidocanol [e.g., polidocanol 3% foam] and pramocaine [pramoxine] [e.g., pramoxine 1% cream]), naturally derived local anesthetics (e.g., cocaine, eugenol, menthol, saxitoxin, neosaxitoxin and tetrodotoxin), and analogs, derivatives and salts thereof;
moisturizers and emollients, including but not limited to aqueous moisturizers, low pH moisturizers containing an acid (e.g., lactic acid), and moisturizers containing a humectant that atm-lets and retains water (e.g., glycerol, sorbitol, lactate, urea, hyaluronic acid and salts thereof, and honey), an occlusive that prevents evaporation (e.g., oils [e.g., mineral oil and silicone oil {e.g., dimethicone.}] and petroleum jelly [petrolatum]), or/and an emollient that provides partial hydration and occlusion (e.g., oils, waxes [e.g., lanolin and paraffin], lipids [e.g., phospholipids, ceramides, triglycerides, glycol stearate, glyceryl stearate, fatty acids and squalene], and sterols [e.g., cholesterol and phytosterol]), and analogs, derivatives and salts thereof and
other kinds of antipruritic agents, including but not limited to allantoin (e.g., 3-6% allantoin cream in SD-101), NST-141, S-adenosyl methionine, endothelin-converting enzyme 1 (ECE-1), botulinum toxin (e.g., botulinum toxin types A and B, which inhibit release of acetylcholine from presynaptic nerve terminals), vitamin D g vitamin D₂ and vitamin D₃) and analogs and derivatives thereof (e.g., calcitriol, calcipotriol [calcipotriene] and paricalcitol), inhibitors of lysophosphatidic acid (LPA) or receptors therefor (e.g., LPAR1 to 6) or the production thereof (e.g., autotaxin inhibitors such as GLPG1690, HA-130, ONO-8430506, PF-8380, S-32826 and anti-autotaxin DNA aptamers [e.g., RB011 and RF3014]), antimicrobials (including antibiotics, antifungals, antivirals and antiparasitics, such as crotamiton and rifampin [rifampicin]), bile acid absorption-reducing, ileal bile acid transporter-inhibiting or bile acid-sequestering agents (e.g., ursodeoxycholic acid [ursodiol], cholestyramine, colestipol and colosevelam), ultraviolet (e.g., ultraviolet A [NA] and ultraviolet B [UVB]) phototherapy, laser therapy, transcutaneous electrical nerve stimulation, acupuncture (using, e.g., electric needles), massage, therapeutic agents that treat the underlying, causes of the pruritus-associated conditions, and analogs, derivatives, fragments and salts thereof.

Examples of non-steroidal anti-inflammatory drugs (NSAIDs) include without limitation:
acetic acid derivatives, such as aceclofenac, bromfenac, diclofenac, etodolac, indomethacin, ketorolac, nabumetone, sulindac, sulindac sulfide, sulindac sulfone and tolmetin;
anthranilic acid derivatives (fenamates), such as flufenamic acid, meclofenamic acid, mefenamic acid and tolfenamic acid;
enolic acid derivatives (oxicams), such as droxicam, isoxicam, lornoxicam, meloxicam, piroxicam and tenoxicam;
propionic acid derivatives, such as fenoprofen, flurbiprofen, ibuprofen, dexibuprofen, ketoprofen, dexketoprofen, loxoprofen, naproxen and oxaprozin;
salicylates, such as diflunisal, salicylic acid, acetylsalicylic acid (aspirin), choline magnesium trisalicylate, and salsalate;
COX-2-selective inhibitors, such as apricoxib, celecoxib, etoricoxib, firocoxib, fluorocoxibs (e.g., fluorocoxibs A-C), lumiracoxib, mavacoxib, parecoxib, rofecoxib, tilmacoxib (JTE-522), valdecoxib, 4-O-methylhonokiol, niflumic acid, DuP-697, CG100649, GW406381, NS-398, SC-58125, benzothieno[3,2-d]pyrimidin-4-one sulfonamide thio-derivatives, and COX-2 inhibitors derived from Tribulus terrestris;
other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) [e.g., chebulagic acid, licofelone, 2-(3,4,5-trimethoxyphenyl)-4-(N-methylindol-3-yl)thiophene, and di-tert-butylphenol-based compounds (e.g., DTPBHZ, DTPINH, DTPNHZ and DTPSAL)]; and
analogs, derivatives and salts thereof.

If desired (e.g., for relief of pruritus during the day), a non-sedating antipruritic agent can be used. For example, second-generation and third-generation H₁ antihistamines are designed to be non-sedating, or less sedating than first-generation H₁ antihistamines, and to affect primarily peripheral H₁ histamine receptors. Non-limiting examples of second-generation and third-generation H₁ antihistamines include acrivastine, astemizole, azelastine, bepotastine, bilastine, cetirizine, cidoxepin, levocetirizine, ebastine, fexofenadine, levocabastine, loratadine, desloratadine, mizolastine, olopatadine, quifenadine, rupatadine, terfenadine, and salts thereof.

A sedating antipruritic agent can also be used, such as at night for relief of pruritus during nighttime. For instance, sedating first-generation H₁ antihistamines that cross the blood-brain barrier can be taken at night to aid with sleep and to decrease nighttime itch and scratching. Non-limiting examples of first-generation H₁ antihistamines include antazoline, azatadine, brompheniramine, buclizine, bromodiphenhydramine (bromazine), carbinoxamine, chlorcyclizine, chloropyramine, chlorpromazine, chlorpheniramine, chlorodiphenhydramine, clemastine, cyclizine, cyproheptadine, dexbrompheniramine, dexchlorpheniramine, dimenhydrinate, dimetindene, diphenhydramine, doxepin, doxylamine, embramine, esmirtazapine, hydroxyzine, ketotifen, meclozine (meclizine), mepyramine, mirtazapine, orphenadrine, phenindamine, pheniramine, phenyltoloxamine, promethazine, pyrilamine, quetiapine, trimeprazine (alimemazine), tripelennamine, triprolidine, and salts thereof.

If a pruritus sufferer has sleep difficulty, which may be caused by pruritus, in addition to or alternative to a sedating antihistamine, the person can take a sedative at night to aid with sleep and to decrease nocturnal itch o/land scratching. Such a sedative can be, e.g., an antidepressant (e.g., a tricyclic antidepressant) or a tranquilizer. A tranquilizer can be a minor tranquilizer (aka an anxiolytic) or a major tranquilizer (aka an antipsychotic or neuroleptic).

In some embodiments, a corticosteroid/glucocorticoid of moderate or medium potency is used in combination with an NK-1 antagonist (e.g., serlopitant) to treat acute or chronic pruritus associated with a condition described herein. Examples of corticosteroids/glucocorticoids having moderate or medium potency include Groups III, IV and V corticosteroids under the 7-group United States classification system and Class II corticosteroids under the 4-class European classification system:

Group III US (upper mid-strength), including but not limited to amcinonide 0.05-0.1% (e.g., Cyclocort® cream/lotion), betamethasone dipropionate 0.05% (e.g., Diprolene® ointment/cream and Diprosone® ointment/cream), betamethasone valerate 0.1% (e.g., ointment and Luxiq® foam), diflorasone diacetate 0.05% (e.g., Florone® cream and Maxiflor® cream), fluocinonide 0.05% (e.g, Lidex-E® cream), fluticasone propionate 0.005% (e.g., Cutivate® ointment), halometasone 0.05% (e.g., cream), mometasone furoate 0.1% (e.g., Elocon® ointment), and triamcinolone acetonide 0.5% (e.g., Aristocort® cream and Kenalog® cream);

Group IV US (mid-strength), including but not limited to desoximetasone 0.05% (e.g., Topicort® LP ointment/cream), fluocinolone acetonide 0.025-0.2% (e.g., Synalar® ointment and Synemol® cream), flurandrenolide 0.05% (e.g., Cordran® ointment), hydrocortisone butyrate 0.1% (e.g., Locoid® ointment/cream), hydrocortisone valerate 0.2% (e.g., Westcort® ointment), mometasone furoate 0.1% (e.g., Elocon® cream/lotion), and triamcinolone acetonide 0.1% (e.g., Aristocort® A ointment and Kenalog® ointment/cream/spray);

Group V US (lower mid-strength), including but not limited to betamethasone dipropionate 0.05% (e.g., Diprosone® lotion), betamethasone valerate 0.1% (e.g., Valisone® cream/lotion), desonide 0.05% (e.g., DesOwen® ointment and Tridesilon® ointment), fluocinolone acetonide 0.025/0.03% (e.g., Synalar® cream and Synemol® cream), fluocinolone acetonide 0.01% (e.g., Synalar® cream), flurandrenolide 0.05% (e.g., Cordran® cream/lotion/tape), fluticasone propionate 0.05% (e.g., Cutivate® cream/lotion), hydrocortisone butyrate 0.1% (e.g., Locoid® cream), hydrocortisone valerate 0.2% (e.g., Westcort® cream), prednicarbate 0.1% (e.g., DermAtop@ cream), and triamcinolone acetonide 0.1% (e.g., Kenalog® lotion); and

Class II EU (moderate), including but not limited to clobetasone butyrate 0.05% (e.g., Eumovate® cream), and triamcinolone acetonide 0.1-0.5% (e.g., Aristocort® ointment/cream, Kenacomb® ointment/cream, Kenalog® cream/spray and Viaderm® KC ointment/cream).

In other embodiments, a potent or very potent corticosteroid/glucocorticoid is used in combination with an NK-1 antagonist (e.g., serlopitant) to treat acute or chronic pruritus associated with a condition described herein. Examples of potent or very potent corticosteroids/glucocorticoids include Groups I and II corticosteroids under the 7-group United States classification system and Classes III and IV corticosteroids under the 4-class European classification system:

Group I US and Class IV EU (very potent), including but not limited to betamethasone dipropionate 0.25% (e.g., Diprolene® ointment/cream, Diprosone® OV ointment/cream and Diprovate® cream), clobetasol propionate 0.05% (e.g., Clobex® lotion/spray, Cormax® cream/solution, Dermovate® ointment/cream, Exel® cream, Olux® foam and Temovate® ointment/cream/solution), desoximetasone 0.25% (e.g., Topicort® topical spray), diflorasone diacetate 0.05% (e.g., Psorcon® ointment), fluocinonide 0.1% (e.g., Vanos® cream), and halobetasol propionate 0.05% (e.g., Halox® lotion and Ultravate® ointment/cream/lotion); and

Group II US and Class III EU (potent), including but not limited to amcinonide 0.05-0.1% (e.g., Cyclocort® ointment), desoximetasone 0.25% (e.g., Topicort® ointment/cream and Topisolon® ointment/cream), desoximetasone 0.05% (e.g., Topicort® gel), diflorasone diacetate 0.05% (e.g, Florone® ointment Maxiflor® ointment and Psorcon® cream), fluocinonide 0.05% (e.g., Lidex® ointment/cream/gel), halcinonide 0.05-0.1% (e.g., Halog® ointment/cream), and halometasone 0.05% (e.g., ointment).

In certain embodiments, a topical corticosteroid of moderate or medium potency or a potent or very potent topical corticosteroid is used for less than, e.g., about 1-2 weeks at a time to decrease side effects such as skin atrophy. For example, a topical corticosteroid can be applied daily (e.g., once daily) for about 3 consecutive days and then not applied for about 3 or 4 consecutive days, and the cycle can be repeated for the duration of the treatment regimen. The treatment regimen of the topical corticosteroid can be based on, e.g., the nature and severity of the pruritus-associated condition, the bodily area(s) affected and the potency of the corticosteroid. If the condition is, e.g., more severe or more generalized, a corticosteroid can also be administered systemically (e.g., orally or parenterally) for a more rapid or more systemic action, although there may be a greater risk of side effects.

In some embodiments, an NK-1 antagonist (e.g., serlopitant) is used in conjunction with an antihistamine, a corticosteroid (e.g., a topical corticosteroid), an immunosuppressant, a kappa-opioid receptor agonist, a mu-opioid receptor antagonist, an anticonvulsant, an antidepressant or UV phototherapy, or any combination thereof, to treat acute or chronic pruritus associated with a medical condition described herein, or/and the medical condition itself.

In some embodiments, an NK-1 antagonist (e.g., serlopitant) and one or more of the following antipruritic or therapeutic agents are used to treat acute or chronic pruritus associated with dermatitis or eczema (e.g., atopic dermatitis) or/and the medical condition itself:
1) in general one or more anti-inflammatory agents that can be administered topically (e.g., dermally or transdermally) or/and systemically (e.g., orally or parenterally); or
2) a topical corticosteroid of moderate or medium potency or a potent or very potent topical corticosteroid, or a systemically (e.g., orally or parenterally) administered corticosteroid for more severe or more widespread dermatitis; or
3) a topical immunosuppressant (e.g., a calcineurin inhibitor such as pimecrolimus [e.g., about 1% pimecrolimus] or tacrolimus [e.g., about 0.1% tacrolimus]), or a systemically (e.g., orally or parenterally) administered immunosuppressant (e.g., mycophenolic acid or a derivative thereof [e.g., mycophenolate mofetil], am antimetabolite such as an antifolate [e.g., methotrexate] or a purine analog [e.g., azathioprine], a calcineurin inhibitor such as ciclosporin, or interferon-gamma) for more severe or more widespread dermatitis; or
4) a PLA2 inhibitor (e.g., ZPL-521), which can be administered topically (e.g., dermally or transdermally) or systemically (e.g., orally); or
5) an NSAID (e.g., aspirin), which can be administered topically (e.g., dermally or transdermally) or systemically (e.g., orally); or
6) an antihistamine (e.g., an H₄ antihistamine such as JNJ-7777120 or ZPL-389, o/land a sedating first-generation H₁ antihistamine such as diphenhydramine for nighttime use), which can be administered topically (e.g., dermally or transdermally) or systemically (e.g., orally); or
7) an inhibitor of a pro-inflammatory cytokine or a receptor therefor or the production thereof (e.g., an inhibitor of IL-2 or IL-2R [e.g., basiliximab or daclizumab], an inhibitor of IL-4 or IL-4R [e.g., dupilumab], an inhibitor of IL-31 or IL-31R [e.g., nemolizumab], or a PDE4 inhibitor such as apremilast or crisaborole), which can be administered topically (e.g., dermally or transdermally) or systemically (e.g., orally or parenterally); or
8) an anti-allergic agent (e.g., tranilast) which be administered, e.g., systemically (e.g., orally or parenterally); or
9) an inhibitor of NGF or a receptor therefor (e.g., a TrkA inhibitor such as CT327), which can be administered topically (e.g., dermally or transdermally) or systemically (e.g., orally or parenterally); or
10) an inhibitor of CGRP or receptor therefor, which can be administered topically (e.g., dermally or transdermally) or systemically (e.g., orally or parenterally); or
11) a PAR2 antagonist (e.g., a tetracycline) or a serine protease inhibitor (e.g., camostat or nafamostat); or
12) an MRGPRX2 antagonist; or
13) an antimuscarinic agent; or
14) botulinum toxin, which can be administered topically (e.g., dermally or transdermally) or parenterally (e.g., subcutaneously); or
15) a mu-opioid receptor antagonist (e.g., naltrexone), which can be administered topically (e.g., dermally or transdermally) or systemically (e.g., orally or parenterally); or
16) a kappa-opioid receptor agonist (e.g., nalfurafine, asimadoline or difelikefalin [CR84.5]); or
17) a cannabinoid receptor agonist (e.g., palmitoylethanolamide [PEA] or S-777469), which can be administered topically (e.g., dermally or transdermally) or systemically (e.g., orally); or
18) an FAAH inhibitor; or
19) an antidepressant (e.g., an SSRI such as fluvoxamine or paroxetine, or a tricyclic antidepressant such as doxepin or cidoxepin), which can be administered topically (e.g., dermally or transdermally) or systemically (e.g., orally); or
20) NST-141, which can be administered, e.g., topically (e.g., dermally or transdermally); or
21) a moisturizer or emollient (e.g., a moisturizer containing an occlusive such as an oil, or a humectant such as urea); or
22) a counterirritant (e.g., capsaicin) or/and a cooling agent (e.g., a local anesthetic or calamine); or
23) a local anesthetic (e.g., polidocanol), which can be administered, e.g., topically (e.g., dermally or transdermally); or
24) vitamin D or an analog or derivative thereof; or
25) a long-chain polyunsaturated fatty acid (e.g., an n-3 [omega-3] fatty acid, such as α-linolenic acid [ALA], eicosapentaenoic acid (EPA) or docosahexaenoic acid [DHA]); or
26) UVB (e.g., narrow-hand UVB such as 311-313 mu) phototherapy or UVA (e.g., UVA1) phototherapy with a skin photosensitizer (e.g., psoralen in PUVA), or
27) any combinations thereof.

In certain embodiments, an NK-1 antagonist (e.g., serlopitant) is used in conjunction with a topical or systemic corticosteroid, a topical or systemic immunosuppressant (e.g., a calcineurin inhibitor), a topical or systemic inhibitor of a pro-inflammatory cytokine or a receptor therefor or the production thereof (e.g., an inhibitor of IL-4 or IL-4R such as dupilumab, an inhibitor of IL-31 or IL-31R such as nemolizumiab, or a PDE4 inhibitor such as apremilast or crisaborole), a topical or systemic antihistamine (e.g., an H₄ antihistamine such as JNJ-7777120 or ZPL-389), a topical or systemic mu-opioid receptor antagonist (e.g., naltrexone), a topical cannabinoid receptor agonist (e.g., PEA), an antidepressant (e.g., an SSRI such as fluvoxamine or paroxetine or a tricyclic antidepressant such as doxepin), a moisturizer or emollient, or UVB phototherapy or UVA phototherapy with a skin photosensitizer (e.g., psoralen), or any combination thereof, to treat acute or chronic pruritus associated with dermatitis or eczema (e.g., atopic dermatitis) or/and the medical condition itself.

In further embodiments, an NK-1 antagonist (e.g., serlopitant) and one or more of the following antipruritic or therapeutic agents are used to treat acute or chronic pruritus associated with psoriasis (e.g., plaque psoriasis) or/and the medical condition itself:
1) in general one or more anti-inflammatory agents that can be administered topically (e.g., dermally or transdermally) or/and systemically (e.g., orally or parenterally); or
2) a topical corticosteroid of moderate or medium potency or a potent or very potent topical corticosteroid; or
3) an immunosuppressant alefacept, mycophenolate mofetil, an antimetabolite such as hydroxyurea, an antifolate [e.g., methotrexate] or a purine analog [e.g., azathioprine or thioguanine], a calcineurin inhibitor such as ciclosporin, an mTOR inhibitor such as rapamycin, or a corticosteroid), which can be administered topically (e.g., dermally or transdermally) or systemically (e.g., orally or parenterally); or
4) an inhibitor of a pro-inflammatory cytokine or a receptor therefor (e.g., an inhibitor of TNT-α [e.g., adalimumab, certolizumab pegol, infliximab or etanercept] or an inhibitor of a pro-inflammatory interleukin or a receptor therefor, such as IL-12 [e.g., ustekinumab] or IL-12R, IL-17 [e.g., ixekizumab or secukinumab] or IL-17R [e.g., brodalumab], IL-20 [e.g., the antibody 7E] or IL-20R, IL-22 [e.g., fezakinumab] or IL-22R, or IL-23 [e.g., guselkumab, risankizumab, tildrakizumab or ustekinumab] or IL-23R), which can be administered, e.g., systemically (e.g., orally or parenterally), or
5) an inhibitor of the production of a pro-inflammatory cytokine or a receptor therefor (e.g., an inhibitor of the production of TNT-α [e.g., a PDE4 inhibitor such as apremilast or crisaborole, a p38 MAP kinase inhibitor such as BMS-582949, or a TLR inhibitor {e.g., a TLR7/TLR9 inhibitor such as IMO-3100}], IL-2 [e.g., a PDE4 inhibitor such as apremilast or crisaborole], IL-6 [e.g., an inhibitor of a TLR such as TLR7 or TLR9], IL-8 [e.g., alefacept], IL-12 [e.g., apilimod], IL-17 [e.g., a PKC inhibitor such as sotrastaurin], or IL-23 [e.g., apilimod or alefacept]), which can be administered topically (e.g., dermally or transdermally) or systemically (e.g., orally or parenterally); or
6) an inhibitor of a pro-inflammatory transcription factor (e.g., an NE-KB inhibitor or a STAT protein inhibitor [e.g., a JAK inhibitor such as tofacitinib]), which can be administered, systemically (e.g., orally or parenterally); or
7) an inhibitor of NGF or a receptor therefor (e.g., a TrkA inhibitor such as CT327), which can be administered topically (e.g., dermally or transdermally) or systemically (e.g., orally or parenterally); or
8) an inhibitor of CGRP or receptor therefor, which can be administered topically (e.g., dermally or transdermally) or systemically (e.g., orally or parenterally); or
9) an inhibitor of CRH or a receptor therefor, which can be administered topically (e.g., dermally or transdermally) or systemically (e.g., orally or parenterally); or
10) an inhibitor of VIP or a receptor therefor, which can be administered topically (e.g., dermally, or transdermally) or systemically (e.g., orally or parenterally); or
11) an inhibitor of somatostatin or a receptor therefor, which can be administered topically (e.g., dermally or transdermally) or systemically (e.g., orally or parenterally); or
12) an antihistamine (e.g., an H₄ antihistamine such as JNJ-7777120 or ZPL-389), which can be administered topically (e.g., dermally or transdermally) or systemically orally); or
13) an inhibitor of a mitogen-activated protein kinase (e.g., a p38 MAP kinase inhibitor such as BMS-582949), which can be administered, e.g., systemically (e.g., orally or parenterally); or
14) an inhibitor of the growth or/and proliferation of cells, including skin cells and immune cells (e.g., a retinoid [e.g., acitretin], an NF-κB inhibitor, a STAT protein inhibitor [e.g., a JAK inhibitor such as tofacitinib], a MAP kinase inhibitor [e.g., a p38 MAP kinase inhibitor], an inhibitor of NGF or a receptor therefor [e.g., a TrkA inhibitor such as CT327], or an inhibitor of a proliferation-inducing cytokine or a receptor therefor or the production thereof [e.g., TNF-α, IFN-α, IL-1, IL-2, IL-7, IL-15, IL-17, IL-20, IL-21, IL-22 or IL-23), which can be administered topically (e.g., dermally or transdermally) or systemically orally or parenterally); or
15) a retinoid (e.g., tazarotene or acitretin), which can be administered topically (e.g., dermally or transdermally) or systemically (e.g., orally or parenterally); or
16) an antioxidant (e.g., a polyphenol, a retinoid, or an activator of nuclear factor (erythroid-derived 2)-like 2 [NFE2L2 or Nrf2] [e.g., a fumarase such as dimethyl fumarate]), which can be administered topically (e.g., dermally or transdermally) or systemically (e.g., orally or parenterally); or
17) an anthrone derivative (e.g., dithranol [anthralin]), which can be administered, e.g., topically (e.g., dermally or transdermally); or
18) vitamin D (e.g., vitamin D₂ or vitamin D₃) or an analog or derivative thereof (e.g., calcitriol, calcipotriol or paricalcitol), which can be administered, e.g., topically (e.g., dermally or transdermally); or
19) a counterirritant (e.g., capsaicin) or/and a cooling agent (e.g., a local anesthetic), which can be administered, e.g., topically (e.g., dermally or transdermally); or
20) a moisturizer or emollient (e.g., a moisturizer containing an occlusive such as mineral oil or petroleum jelly, or a humectant such as urea); or
21) coal tar; or
22) UVB (e.g., narrow-band UVB such as 311-313 nm) phototherapy or UVA phototherapy with a skin photosensitizer (e.g., psoralen in PUVA); or
23) laser (e.g., excimer laser) therapy; or
24) any combinations thereof.

In some embodiments, an NK-1 antagonist (e.g., serlopitant) is used in combination with one or more topical agents to treat relatively mild psoriasis or/and pruritus associated therewith, with ultraviolet phototherapy to treat moderate psoriasis or/and pruritus associated therewith, and with one or more systemic agents to treat severe psoriasis or/and pruritus associated therewith, although topical agents and UV phototherapy can also be used to treat more severe psoriasis or/and pruritus associated therewith, and systemic agents can also be used to treat less severe psoriasis or/and pruritus associated therewith. In certain embodiments, an NK-1 antagonist (e.g., serlopitant) is used in conjunction with a topical corticosteroid (e.g., desoximetasone or fluocinonide), a topical anthrone derivative (e.g., dithranol), a topical vitamin D (e.g., vitamin D₂ or vitamin D₃) or an analog or derivative thereof (e.g., calcitriol, calcipotriol or paricalcitol), a topical or systemic retinoid (e.g., tazarotene or acitretin), a moisturizer or emollient, UVB phototherapy or UVA phototherapy with a skin photosensitizer (e.g., psoralen), a topical or systemic immunosuppressant alefacept, hydroxyurea, methotrexate or ciclosporin), a systemic inhibitor of a pro-inflammatory cytokine or a receptor therefor (e.g., an inhibitor of TNT-α, a pro-inflammatory interleukin or a receptor therefor, such as adalimumab, infliximab, etanercept, ixekizumab, secukinumab, brodalumab, tildrakizumab or ustekinumab), or a topical or systemic inhibitor of the production of a pro-inflammatory cytokine or a receptor therefor (e.g., apilimod, a PDE4 inhibitor such as apremilast or crisaborole, a JAK inhibitor such as tofacitinib, or a PKC inhibitor such as sotrastaurin), or any combination thereof, to treat acute or chronic pruritus associated with psoriasis (e.g., plaque psoriasis) or/and the medical condition itself.

In additional embodiments, an NK-1 antagonist (e.g., serlopitant) and one or more of the following antipuritic or therapeutic agents are used to treat acute or chronic pruritus associated with prurigo (e.g., prurigo nodularis) or/and the medical condition itself:
1) a topical corticosteroid (e.g., betamethasone or a derivative thereof) of moderate or medium potency to high or very high potency, or a systemically (e.g., orally or parenterally) administered corticosteroid (e.g., prednisone or a derivative thereof); or
2) a topical immunosuppressant (e.g., a calcineurin inhibitor such as pimecrolimus or tacrolimus), or a systemically (e.g., orally or parenterally) administered immunosuppressant (e.g., an antimetabolite such as an antifolale [e.g., methotrexate] or a purine analog [e.g., azathioprine], or a calcineurin inhibitor such as ciclosporin), or
3) an immunomodulator (e.g., an imide such as thalidomide), which can be administered, e.g., systemically (e.g., orally or parenterally); or
4) an inhibitor of a pro-inflammatory cytokine or a receptor therefor or the production thereof (e.g., an antibody targeting IL-31 or IL-31R such as nemolizumab); or
5) an anti-allergic agent (e.g., tranilast), which can be administered, e.g., systemically (e.g., orally or parenterally); or
6) an antihistamine (e.g., loratadine or cetirizine), which can be administered topically (e.g, dermally or transdermally) or systemically (e.g., orally or parenterally); or
7) an inhibitor of CGRP or receptor therefor, which can be administered topically (e.g., dermally or transdermally) or systemically (e.g., orally or parenterally); or
8) an inhibitor of NGF or a receptor therefor (e.g., a TrkA inhibitor such as CT327), which can be administered topically (e.g, dermally or transdermally) or systemically (e.g., orally or parenterally); or
9) a mu-opioid receptor antagonist (e.g., naltrexone), which can be administered topically (e.g., dermally or transdermally) or systemically (e.g., orally or parenterally); or
10) a kappa-opioid receptor agonist (e.g., nalfurafine, asimadoline, difelikefalin [CR845] or nalbuphine), which can be administered, e.g., systemically (e.g., orally or parenterally); or
11) a cannabinoid receptor agonist (e.g., palmitoylethanolamide or S-777469), which can be administered topically (e.g., dermally or transdermally) or systemically (e.g., orally); or
12) an anticonvulsant (e.g., gabapentin or pregabalin), which can be administered, e.g., systemically (e.g., orally or parenterally); or
13) an antidepressant (e.g., a tricyclic antidepressant such as amitriptyline, doxepin or cidoxepin, or an SSRI such as fluvoxamine or paroxetine), which can be administered topically (e.g., dermally or transdermally) or systemically (e.g., orally or parenterally); or
14) a local anesthetic (e.g., polidocanol), which can be administered, e.g., topically (e.g., dermally or transdermally); or
15) a moisturizer or emollient, or
16) a counterirritant (e.g., capsaicin) or/and a cooling agent (e.g., a local anesthetic), or a substance that is both a counterirritant and a cooling agent (e.g., camphor or menthol), which can be administered, e.g., topically de III tally or transdermally); or
17) vitamin D (e.g., vitamin D₃) or an analog or derivative thereof, which can be administered, e.g., topically (e.g., dermally or transdermally); or
18) UVB (e.g., narrow-hand UVB such as 311-313 nm) phototherapy or UVA phototherapy with a skin photosensitizer psoralen in PUVA); or
19) any combinations thereof.

In some embodiments, an NK-1 antagonist (e.g., serlopitant) is used in conjunction with a topical or systemic corticosteroid (e.g., betamethasone, prednisone or a derivative thereof), an immunomodulator (e.g., thalidomide), a topical or systemic immunosuppressant (e.g., a calcineurin inhibitor such as pimecrolimus, tacrolimus or ciclosporin, or an antimetabolite such as methotrexate or azathioprine), an antihistamine (e.g., loratadine or cetirizine), a topical or systemic mu-opioid receptor antagonist (e.g., naltrexone), an anticonvulsant (e.g., gabapentin or pregabalin), an antidepressant (e.g., a tricyclic antidepressant such as amitriptyline or doxepin, or an SSRI such as fluvoxamine or paroxetine), a topical counterirritant (e.g., capsaicin) or/and a cooling agent (e.g., a local anesthetic), or UVB phototherapy or UVA phototherapy with a skin photosensitizer (e.g., psoralen), or any combination thereof, to treat acute or chronic pruritus associated with prurigo (e.g., prurigo nodularis) or/and the medical condition itself. In certain embodiments, an NK-1 antagonist (e.g., serlopitant) is used in combination with an antihistamine (e.g., loratadine or cetirizine) to treat acute or chronic pruritus associated with prurigo (e.g., prurigo nodularis) or/and the medical condition itself.

In other embodiments, an NK-1 antagonist (e.g., serlopitant) and one or more of the following antipruritic or therapeutic agents are used to treat acute or chronic pruritus associated with urticaria (e.g., chronic idiopathic urticaria) or/and the medical condition itself:
1) in general one or more anti-inflammatory agents that can be administered topically (e.g., dermally or transdermally) or/and systemically (e.g., orally or parenterally); or
2) an antihistamine (e.g., a second-generation H₁ antihistamine such as cetirizine, cidoxepin, loratadine or desloratadine, or/and a first-generation H₁ antihistamine such as diphenhydramine, doxepin or hydroxyzine, and optionally an H₂ antihistamine such as cimetidine [e.g., cidoxepin or/and hydroxy zinc, or hydroxyzine and cimetidine]), which can be administered topically (e.g., dermally or transdermally) or systemically (e.g., orally or parenterally); or
3) an inhibitor of a leukotriene or a receptor therefor or the production thereof (e.g., a leukotriene receptor antagonist such as montelukast or zafirlukast); or
4) a mast cell stabilizer (e.g., ketotifen); or
5) a glucocorticoid, which can be administered topically (e.g., dermally or transdermally) or systemically (e.g., orally or parenterally); or
6) an inhibitor of a pro-inflammatory cytokine or a receptor therefor or the production thereof (e.g., a TLR9 inhibitor such as hydroxychloroquine); or
7) a myeloperoxidase inhibitor (e.g., dapsone); or
8) an IgE inhibitor (e.g., an anti-IgE antibody such as omalizumab); or
9) a DMARD (e.g., sulfasalazine); or
10) an anti-allergic agent; or
11) an immunosuppressant (e.g., mycophenolate, a calcineurin inhibitor such as cyclosporine or tacrolimus, or an mTOR inhibitor such as rapamycin); or
12) a PAR2 antagonist (e.g., a tetracycline) or a serine protease inhibitor (e.g., camostat or nafamostat); or
13) an MRGPRX2 antagonist; or
14) a moisturizer or emollient; or
15) a counterirritant (e.g., capsaicin) or/and a cooling agent (e.g., a local anesthetic or calamine); or
16) UVB (e.g., narrow-band UVB such as 311-313 nm) phototherapy or UVA phototherapy with a skin photosensitizer (e.g., psoralen in PUVA); or
17) any combinations thereof.

In some embodiments, an NK-1 antagonist (e.g., serlopitant) is used in conjunction with a topical or systemic antihistamine (e.g., a second-generation H₁ antihistamine such as cetirizine, cidoxepin, loratadine or desloratadine, or/and a first-generation H₁ antihistamine such as diphenhydramine, doxepin or hydroxyzine, and optionally an H₂ antihistamine such as cimetidine [e.g., cidoxepin or/and hydroxy zinc, or hydroxyzine and cimetidine]), an inhibitor of a leukotriene or a receptor therefor or the production thereof (e.g., a leukotriene receptor antagonist such as montelukast or zafirlukast), a topical or systemic glucocorticoid, an IgE inhibitor (e.g., an anti-IgE antibody such as omalizumab), a DMARD (e.g., sulfasalazine), an immunosuppressant (e.g., mycophenolate, a calcineurin inhibitor such as cyclosporine or tacrolimus, or an mTOR inhibitor such as rapamycin), or another kind of anti-inflammatory agent (e.g., dapsone o/land hydroxychloroquine), or any combination thereof, to treat acute or chronic pruritus associated with urticaria (e.g., chronic idiopathic urticaria) or/and the medical condition itself. In certain embodiments, an NK-1 antagonist (e.g., serlopitant) is used in combination with one or more antihistamines (including, e.g., an H₁ antihistamine) to treat acute or chronic pruritus associated with urticaria (e.g., chronic idiopathic urticaria) or/and the medical condition itself.

In some embodiments, an NK-1 antagonist (e.g., serlopitant) and one or more of the following antipruritic or therapeutic agents are used to treat acute or chronic pruritus associated with cutaneous T-cell lymphoma (CTCL) (e.g., mycosis fungoides) or/and the medical condition itself:
1) a mu-opioid receptor antagonist (e.g., naloxone), which can be administered topically (e.g., dermally or transdermally) or systemically (e.g., orally or parenterally); or
2) a corticosteroid, which can be administered topically (e.g., dermally or transdermally) or systemically (e.g., orally or parenterally); or
3) an immunosuppressant (e.g., an antimetabolite such as an antifolate [e.g., methotrexate] or a purine analog [e.g., azathioprine]); or
4) an immune-response modifier (e.g., gardiquimod, imiquimod or resiquimod), which can be administered, e.g., topically (e.g., dermally or transdermally), or
5) an inhibitor of NGF or a receptor therefor (e.g., a TrkA inhibitor such as CT327), which can be administered topically (e.g., dermally or transdermally) or systemically (e.g., orally or parenterally); or
6) an antihistamine (e.g., an H₄ antihistamine); or
7) a serotonin receptor antagonist; or
8) an antidepressant (e.g., an SSRI such as paroxetine or a tetracyclic antidepressant such as mirtazapine or esmirtazapine); or
9) a moisturizer or emollient; or
10) an anti-cancer agent (e.g., a retinoid X receptor agonist such as a retinoid [e.g., bexarotene], or a histone deacetylase inhibitor [e.g., panobinostat, vorinostat or romidepsin]), which can be administered topically (e.g., dermally or transdermally) or systemically (e.g., orally or parenterally); or
11) superficial radiation therapy, which can be local or generalized; or
12) UVB (e.g., narrow-band [e.g., 311-313 nm] or broad-band [e.g., 280-315 nm] UVB) phototherapy or UVA phototherapy with a skin photosensitizer (e.g., psoralen in PUVA); or
13) any combinations thereof.

In certain embodiments, an NK-1 antagonist (e.g., serlopitant) is used in conjunction with a mu-opioid receptor antagonist (e.g., naloxone), a corticosteroid, an immune-response modifier (e.g., resiquimod), an anti-cancer agent (e.g., bexarotene or vorinostat), or UVB phototherapy or UVA phototherapy with a skin photosensitizer psoralen), or any combination thereof, to treat acute or chronic pruritus associated with CTCL (e.g., mycosis fungoides) or/and the medical condition itself.

In further embodiments, an NK-1 antagonist (e.g., serlopitant) and one or more of the following antipruritic or therapeutic agents are used to treat acute or chronic pruritus associated with epidermolysis bullosa (EB) (e.g., EB simplex) or/and the medical condition itself:
1) allantoin, which can be administered, e.g., topically (e.g., dermally or transdermally, such as 3-6% allantoin cream in SD-101); or
2) a sulfinyl isothiocyanate (e.g., sulforaphane or raphanin), which can be administered topically (e.g., dermally or transdermally) or systemically (e.g., orally or parenterally); or
3) granulocyte-colony stimulating factor (G-CSF), which can be administered, e.g., systemically (e.g., orally or parenterally); or
4) a corticosteroid, which can be administered topically (e.g., dermally or transdermally) or systemically (e.g., orally or parenterally); or
5) an immunosuppressant for an autoimmune type of EB (e.g., EB acquisita), which can be administered topically (e.g., dermally or transdermally) or systemically (e.g., orally or parenterally); or
6) an antidepressant (e.g., a tricyclic antidepressant such as doxepin or cidoxepin), which can be administered topically (e.g., dermally or transdermally) or systemically (e.g., orally or parenterally); or
7) a moisturizer or emollient (e.g., a moisturizer containing an occlusive such as petroleum jelly); or
8) photopheresis; or
9) any combinations thereof.

In additional embodiments, an NK-1 antagonist (e.g., serlopitant) and one or more of the following antipruritic or therapeutic agents are used to treat acute or chronic pruritus associated with a burn, such as a thermal burn, a second-degree burn or a third-degree burn, or a moderate burn or a major burn:
1) an antihistamine (e.g., an H₁ antihistamine such as chlorpheniramine, diphenhydramine or hydroxyzine) which can be administered topically (e.g., dermally or transdermally) or systemically (e.g., orally or parenterally); or
2) an anticonvulsant (e.g., gabapentin), which can be administered, e.g., systemically (e.g., orally or parenterally); or
3) a mu-opioid receptor antagonist (e.g., naltrexone), which can be administered topically (e.g., dermally or transdermally) or systemically (e.g., orally or parenterally); or
4) a corticosteroid, which can be administered, e.g., topically (e.g., dermally or transdermally); or
5) a counterirritant (e.g., capsaicin) or/and a cooling agent (e.g., a local anesthetic or calamine), or a substance that is both a counterirritant and a cooling agent (e.g., camphor or menthol), which can be administered, e.g., topically (e.g., dermally or transdermally); or
6) a moisturizer or emollient (e.g., a moisturizer containing a humectant such as honey, an occlusive such as silicone gel, or colloidal oatmeal); or
7) UVB (e.g., narrow-band UVB such as 311-313 n) phototherapy or UVA phototherapy with a skin photosensitizer (e.g., psoralen in PUVA); or
8) laser therapy; or
9) transcutaneous electrical nerve stimulation; or
10) massage; or
11) any combinations thereof.

In certain embodiments, an NK-1 antagonist (e.g., serlopitant) is used in conjunction with an antihistamine (e.g., an H₁ antihistamine such as chlorpheniramine, diphenhydramine or hydroxy zinc), an anticonvulsant (e.g., gabapentin), a mu-opioid receptor antagonist (e.g., naltrexone), or a moisturizer or emollient, or any combination thereof, to treat acute or chronic pruritus associated with a burn, such as a thermal burn, a second-degree burn or a third-degree burn, or a moderate burn or a major burn.

In other embodiments, an NK-1 antagonist (e.g., serlopitant) and one or more of the following antipruritic or therapeutic agents are used to treat acute or chronic pruritus associated with a hepato-biliary disease e.g., a cholestatic disorder such as cholestasis or primary biliary cirrhosis [PBC]) o/land the medical condition itself:
1) a bile acid-/bile salt-chelating or -sequestering agent (e.g., an ion-exchange resin such as cholestyramine); or
2) a cholesterol absorption-reducing or gallstone-dissolving agent (e.g., ursodeoxycholic acid [ursodiol] or chenodeoxycholic acid); or
3) an agonist of the farnesoid X receptor (FXR, aka bile acid receptor) (e.g., cafestol, chenodeoxycholic acid, obeticholic acid or fexaramine); or
4) an inhibitor of lysophosphatidic acid (LPA) or a receptor therefor or the production thereof (e.g., an autotaxin inhibitor); or
5) a mu-opioid receptor antagonist (e.g., nalmefene, naloxone or naltrexone); or
6) a kappa-opioid receptor agonist (e.g., nalfurafine, asimadoline or difelikefalin [CR845]); or
7) an antidepressant (e.g, an SSRI such as paroxetine or a tetracyclic antidepressant such as mirtazapine); or
8) a serotonin receptor antagonist (e.g., a 5-HT₃ antagonist such as ondansetron or mirtazapine); or
9) an antihistamine; or
10) a glucocorticoid (e.g., prednisone) for, e.g., an inflammatory or autoimmune hepato-biliary disease (e.g., autoimmune hepatitis or PBC); or
11) an immunosuppressant (e.g., an antimetabolite such as a purine analog [e g, azathioprine] or a calcineurin inhibitor such as ciclosporin) for, e.g., an inflammatory or autoimmune hepato-biliary disease (e.g., autoimmune hepatitis or PBC); or
12) a copper-chelating agent (e.g., penicillamine) for a hepato-biliary disease in which copper accumulates in the liver (e.g., Wilson's disease or cirrhosis caused thereby); or
13) an antiviral drug for a hepato-biliary disease caused by a virus (e.g., viral hepatitis such as hepatitis B or C); or
14) S-adenosyl methionine; or
15) rifampicin; or
16) stanozolol; or
17) one or more vitamins (e.g., vitamin A, D, E or K, or any combination or all thereof); or
18) phototherapy (e.g., bright-light therapy, UVB [e.g., narrow-band UVB such as 311-313 nm] phototherapy or UVA phototherapy with a skin photosensitizer [e.g., psoralen in PUVA]); or
19) any combinations thereof.

In some embodiments, an NK-1 antagonist (e.g., serlopitant) is used in conjunction with a bile acid-/bile salt-chelating or -sequestering agent (e.g., an ion-exchange resin such as cholestyramine), cholesterol absorption-reducing or gallstone-dissolving agent (e.g., ursodeoxycholic acid or chenodeoxycholic acid), an FXR agonist (e.g., cafestol, chenodeoxycholic acid, obeticholic acid or fexaramine), an inhibitor of LPA or a receptor therefor or the production thereof (e.g., an autotaxin inhibitor), a mu-opioid receptor antagonist (e.g., nalmefene, naloxone or naltrexone), or an antidepressant (e.g., an SSRI such as paroxetine or a tetracyclic antidepressant such as mirtazapine), or any combination thereof, to treat acute or chronic pruritus associated with a hepato-biliary disease (e.g., a cholestatic disorder such as cholestasis or PBC) or/and the medical condition itself. In certain embodiments, an NK-1 antagonist (e.g., serlopitant) is used in combination with obeticholic acid or/and ursodeoxycholic acid to treat acute or chronic pruritus associated with a cholestatic disorder (e.g., cholestasis or PBC) or/and the medical condition itself.

The optional additional antipruritic or therapeutic agent(s) can be administered to a subject suffering from acute or chronic pruritus associated with a condition described herein concurrently with (e.g., in the same composition as the NK-1 antagonist or in separate compositions) or sequentially to (before or after) administration of the NK-1 antagonist (e.g., serlopitant). The NK-1 antagonist (e.g., serlopitant) and the optional additional antipruritic or therapeutic agent(s) independently can be administered in any suitable mode, including without limitation orally, topically (e.g., dermally/epicutaneously, transdermally, mucosally, transmucosally, intranasally [e.g., by nasal spray or drop], opthalmically [e.g., by eye drop], pulmonarily [e.g., by oral or nasal inhalation], bucally, sublingually, rectally and vaginally), by injection or infusion (e.g., parenterally, including intramuscularly, subcutaneously, intradermally, intravenously/intravascularly, and intrathecally), and by implantation (e.g., subcutaneously and intramuscularly). In some embodiments, an antipruritic or therapeutic agent is administered topically (e.g., dermally or transdermally) if the pruritus or the pruritus-associated condition is localized o/land less severe, and is administered systemically (e.g., orally or intravenously) if the pruritus or the pruritus-associated condition is widespread (generalized), has a systemic cause or/and is more severe. In certain embodiments, the NK-1 antagonist (e.g., serlopitant) or/and the optional additional antipruritic or therapeutic agent(s) (e.g., ciclosporin, an antihistamine, an anticonvulsant, an antidepressant, or an opioid receptor antagonist or agonist) are administered systemically (e.g., orally). In other embodiments, the NK-1 antagonist (e.g., serlopitant) or/and the optional additional antipruritic or therapeutic agent(s) (e.g., a counterirritant such as capsaicin, a calcineurin inhibitor such as pimecrolimus or tacrolimus, or a cannabinoid agonist such as PEA) are administered topically (e.g., dermally or transdermally).

The NK-1 antagonist (e.g., serlopitant) and the optional additional antipruritic or therapeutic agent(s) independently can be administered in any suitable frequency, including without limitation daily (one, two, three or more times per day), every two or three days, twice weekly, thrice weekly, weekly, every two weeks, every three weeks, monthly, every two months and every three months. The dosing frequency can depend on, e.g., the mode of administration chosen. For example, a dermal formulation of the NK-1 antagonist (e.g serlopitant), or/and that of the optional additional antipruritic or therapeutic agent(s), can be applied to the skin of a subject one, two, three, four or more times a day. In some embodiments, the NK-1 antagonist (e.g, serlopitant), and optionally the optional additional antipruritic or therapeutic agent(s), are administered over a period of at least about 2 weeks, 1 month (4 weeks), 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).

Examples of topical dosage forms include without limitation creams, ointments, gels, liniments, lotions, suppositories g rectal and vaginal suppositories), buccal and sublingual tablets and pills, sprays (e.g., dermal and nasal sprays), and drops (e.g., eye, nose and ear drops). Non-limiting examples of oral dosage forms include solid dosage forms (e.g., tablets, capsules, pills and cachets) and liquid dosage forms (e.g., solutions or suspensions in an aqueous liquid or/and a non-aqueous liquid, and oil-in-water liquid emulsions or water-in-oil liquid emulsions). In a non-limiting example of a formulation for injection, the formulation is in the form of a solution and comprises an antipruritic or therapeutic agent (e.g., a local anesthetic), a vehicle (e.g., a water-based vehicle or sterile water), a buffer, a reducing agent/antioxidant (e.g., sodium metabisulfite if epinephrine is used as a vasoconstrictor) and a preservative (e.g., methylparaben), and optionally a vasoconstrictor (e.g., epinephrine) to increase the duration of the pharmacological effect of the antipruritic or therapeutic agent by constricting the blood vessels, thereby concentrating the antipruritic or therapeutic agent for an extended duration and increasing the maximum dose of the antipruritic or therapeutic agent.

### Representative Embodiments

The following embodiments of the disclosure are provided by way of illustration and example:

1. A method of treating pruritus associated with dermatitis/eczema, psoriasis, prurigo, urticaria, cutaneous T-cell lymphoma, epidermolysis bullosa, a burn or a hepato-biliary disease, or/and treating the medical condition itself, comprising administering to a subject in need of treatment a therapeutically effective amount of a neurokinin-1 (NK-1) antagonist.
2. The method of embodiment 1, wherein the NK-1 antagonist is or comprises a selective NK-1 antagonist.
3. The method of embodiment 1 or 2, wherein the NK-1 antagonist is selected from aprepitant (L-754030 or MK-869), fosaprepitant (L-758298), befetupitant, casopitant (GW-679769), dapitant (RPR-100893), ezlopitant (CJ-11974), lanepitant (LY-303870), maropitant (0-11972), netupitant, nolpitantium (SR-140333), orvepitant (GW-823296), rolapitant, serlopitant, tradipitant (VIA-686 or LY-686017), vestipitant (GW-597599), vofopitant (GR-205171), hydroxyphenyl propamidobenzoic acid, maltooligosaccharides (e.g., maltotetraose and maltopentaose), spantides (e.g., spantide I and II), AV-608, AV-818, ALD-2624, BIIF 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-11.6031, R-116301, RP-67580, SCH-206272, SCH-388714, SCH-900978, SLV-317, SSR-240600, T-2328, TA-5538, TAK-637, TKA-731, ZD-4974, ZD-6021, and analogs, derivatives, prodrugs, metabolites, salts and combinations thereof.
4. The method of any one of the preceding embodiments, wherein the NK-1 antagonist is or comprises serlopitant, or a pharmaceutically acceptable salt, solvate, hydrate, clathrate, polymorph, prodrug or metabolite thereof.
5. The method of any one of the preceding embodiments, wherein the therapeutically effective amount of the NK-1 antagonist (e.g., serlopitant) is about 0.1-200 mg, 0.1-150 mg, 0.1-100 mg, 0.1-50 mg, 0.1-30 mg, 0.5-20 mg, 0.5-10 mg or 1-10 mg (e.g., per day or per dose).
6. The method of embodiment 5, wherein the therapeutically effective amount of the NK-1 antagonist (e.g, serlopitant) is about 0.5-5 mg, 1-5 mg or 5-10 mg, or about 0.5 mg, 1 mg, 5 mg or 10 mg (e.g., about 5 mg) (e.g., per day or per dose).
7. The method of any one of the preceding embodiments, wherein the therapeutically effective amount of the NK-1 antagonist (e.g., serlopitant) is administered one or more (e.g., two) times a day, or once every two or three days, or once, twice or thrice a week.
8. The method of embodiment 7, wherein the therapeutically effective amount of the NK-1 antagonist (e.g., serlopitant) is administered once daily.
9. The method of any one of the preceding embodiments, wherein the therapeutically effective amount of the NK-1 antagonist (e.g., serlopitant) is administered over a period of at least about 2 weeks, 1 month (4 weeks), 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).
10. The method of any one of the preceding embodiments, wherein the NK-1 antagonist (e.g., serlopitant) is administered orally, parenterally (e.g., intravenously, subcutaneously or intradermally), or topically (e.g., dermally/epicutaneously, transdermally, mucosally, transmucosally, buccally or sublingually).
11. The method of embodiment 10, wherein the NK-1 antagonist (e.g., serlopitant) is administered orally (e.g., as a tablet or capsule) or topically (e.g., dermally or transdermally).
12. The method of any one of the preceding embodiments, wherein the NK-1 antagonist (e.g., serlopitant) is administered in a dose of about 0.5, 1, 5 or 10 mg (e.g., about 5 mg) orally (e.g., as a tablet) once daily for at least about 2 weeks, 1 month, 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).
13. The method of any one of the preceding embodiments, wherein east one loading dose of the NK-1 antagonist (e.g., serlopitant) is first administered, and at least one therapeutically effective maintenance dose of the NK-1 antagonist is subsequently administered.
14. The method of embodiment 13, wherein the at least one therapeutically effective maintenance dose of the NK-1 antagonist (e.g., serlopitant) is about 0.1-200 mg, 0.1-150 mg, 0.1-100 mg, 0.1-50 mg, 0.1-30 rag, 0.5-20 mg, 0.5-10 mg or 1-10 mg (e.g., about 0.5-5 tug, 1-5 mg or 5-10 mg) (e.g., per day or per dose).
15. The method of embodiment 13 or 14, wherein the at least one loading dose of the NK-1 antagonist (e.g., serlopitant) is about 1.5, 2, 3, 4 or 5 times (e.g., about 3 times) greater than the at least one therapeutically effective maintenance dose of the NK-1 antagonist.
16. The method of any one of embodiments 13 to 15, wherein the at least one therapeutically effective maintenance dose of the NK-1 antagonist (e.g, serlopitant) is administered one or more (e.g., two) times a day, or once every two or three days, or once, twice or thrice a week (e.g., once daily).
17. The method of any one of embodiments 13 to 16, wherein the at least one therapeutically effective maintenance dose of the NK-1 antagonist (e.g, serlopitant) is administered over a period of at least about 2 weeks, 1 month, 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months).
18. The method of any one of embodiments 13 to 17, wherein the NK-1 antagonist (e.g., serlopitant) is administered in a loading dose of about 1.5, 3, 15 or 30 mg (e.g., 3×about 0.5, 1, 5 or 10 mg) orally (e.g., as a tablet) on day 1, followed by a maintenance dose of about 0.5, 1, 5 or 10 mg orally (e.g., as a tablet) once daily (e.g., a loading dose of about 15 mg on day 1 followed by a maintenance dose of about 5 mg once daily) for at least about 2 weeks, 1 month, 6 weeks, 2 mouths, 10 weeks, 3 months, 4 months, 5 months, 6 months, 1 year, 1.5 years, 2 years, 3 years or longer (e.g., at least about 6 weeks, 2 months, 3 months or 6 months).
19. The method of any one of the preceding, embodiments, wherein the NK-1 antagonist (e.g., serlopitant) is administered at bedtime.
20. The method of any one of the preceding embodiments, wherein the NK-1 antagonist (e.g., serlopitant) is administered without food (e.g., at least about 1 or 2 hours before or after a meal, such as at least about 2 hours after an evening meal).
21. The method of any one of the preceding embodiments, wherein the pruritus is chronic pruritus or/and the medical condition is chronic.
22. The method of any one of the preceding embodiments, wherein the pruritus is associated with, or/and the medical condition is, dermatitis or eczema.
23. The method of embodiment 22, wherein the dermatitis or eczema is atopic dermatitis.
24. The method of any one of embodiments 1 to 21, wherein the pruritus is associated with, or/and the medical condition is, psoriasis.
25. The method of embodiment 24, wherein the psoriasis is plaque psoriasis (aka psoriasis vulgaris).
26. The method of any one of embodiments 1 to 21, wherein the pruritus is associated with, or/and the medical condition is, prurigo.
27. The method of embodiment 26, wherein the prurigo is prurigo nodularis.
28. The method of any one of embodiments 1 to 21, wherein the pruritus is associated with, or/and the medical condition is, urticaria.
29. The method of embodiment 28, wherein the urticaria is chronic idiopathic urticaria.
30. The method of any one of embodiments 1 to 21, wherein the pruritus is associated with, or/and the medical condition is, cutaneous T-cell lymphoma (CTCL).
31. The method of embodiment 30, wherein the CTCL is mycosis fungoides or a form or variant thereof (e.g., erythrodermic mycosis fungoides, granulomatous slack skin, pagetoid reticulosis or Sezary syndrome).
32. The method of any one of embodiments 1 to 21, wherein the pruritus is associated with, or/and the medical condition is, epidermolysis bullosa (EB).
33. The method of embodiment 32, wherein the EB is EB simplex.
34. The method of any one of embodiments 1 to 21, wherein the pruritus is associated with a burn or post-burn pruritus.
35. The method of embodiment 34, wherein the burn is a thermal burn, a second-degree burn or a third-degree burn, or a moderate burn or a major burn.
36. The method of any one of embodiments 1 to 21, wherein the pruritus is associated with, o/land the medical condition is, a hepato-biliary disease.
37. The method of embodiment 36, wherein the pruritus is cholestatic pruritus or is associated with, or/and the medical condition is, a cholestatic disorder (e.g., cholestasis or primary biliary cirrhosis).
38. The method of any one of the preceding embodiments, further comprising administering one or more additional antipruritic or therapeutic agents.
39. The method of embodiment 38, wherein the one or more additional antipruritic or therapeutic agents are or comprise an antihistamine, a corticosteroid (e.g., a topical corticosteroid), an immunosuppressant, a kappa-opioid receptor agonist, a mu-opioid receptor antagonist, an anticonvulsant, an antidepressant or UV phototherapy, or any combination thereof.
40. The method of embodiment 38 or 39, wherein the pruritus is associated with, or/and the medical condition is, dermatitis or eczema (e.g., atopic dermatitis), and the one or more additional antipruritic or therapeutic agents are or comprise:
1) in general one or more anti-inflammatory agents that can be administered topically (e.g., dermally or transdermally) or/and systemically (e.g., orally or parenterally); or
2) a topical corticosteroid of moderate or medium potency or a potent or very potent topical corticosteroid, or a systemically (e.g., orally or parenterally) administered corticosteroid for more severe or more widespread dermatitis; or
3) a topical immunosuppressant (e.g., a calcineurin inhibitor such as pimecrolimus [e.g., about 1% pimecrolimus] or tacrolimus [e.g., about 0.1% tacrolimus]), or a systemically (e.g., orally or parenterally) administered immunosuppressant (e.g., mycophenolic acid or a derivative thereof [e.g., mycophenolate mofetil], an antimetabolite such as an antifolate [e.g., methotrexate] or a purine analog [e.g., azathioprine], a calcineurin inhibitor such as ciclosporin, or interferon-gamma) for more severe or more widespread dermatitis; or
4) a PLA2 inhibitor (e.g., ZPL-521), which can be administered topically (e.g., dermally or transdermally) or systemically (e.g., orally); or
5) an NSAID (e.g., aspirin), which can be administered topically (e.g., dermally or transdermally) or systemically (e.g., orally); or
6) an antihistamine (e.g., an H₄ antihistamine such as NJ-7777120 or ZPL-389, or/and a sedating first-generation H₁ antihistamine such as diphenhydramine for nighttime use), which can be administered topically (e.g., dermally or transdermally) or systemically (e.g., orally); or
7) an inhibitor of a pro-inflammatory cytokine or a receptor therefor or the production thereof (e.g., an inhibitor of IL-2 or IL-2R [e.g., basiliximab or daclizumab], an inhibitor of IL-4 or IL-4R [e.g., dupilumab], an inhibitor of IL-31 or IL-31R [e.g., nemolizumab], or a PDE4 inhibitor such as apremilast or crisaborole), which can be administered topically (e.g., dermally or transdermally) or systemically (e.g., orally or parenterally); or
8) an anti-allergic agent (e.g., tranilast), which can be administered, e.g., systemically (e.g., orally or parenterally); or
9) an inhibitor of NGF or a receptor therefor (e.g., a TrkA inhibitor such as CT327), which can be administered topically (e.g., dermally or transdermally) or systemically (e.g., orally or parenterally); or
10) an inhibitor of CGRP or receptor therefor, which can be administered topically (e.g., dermally or transdermally) or systemically (e.g., orally or parenterally); or
11) a PAR2 antagonist (e.g., a tetracycline) or a serine protease inhibitor (e.g., camostat or nafamostat); or
12) an MRGPRX2 antagonist; or
13) an antimuscarinic agent; or
14) botulinum toxin, which can be administered topically (e.g, dermally or transdermally) or parenterally (e.g., subcutaneously); or
15) a mu-opioid receptor antagonist (e.g., naltrexone), which can be administered topically (e.g., dermally or transdermally) or systemically (e.g., orally or parenterally); or
16) a kappa-opioid receptor agonist (e.g., nalfurafine, asimadoline or difelikefalin [CR845]); or
17) a cannabinoid receptor agonist (e.g., palmitoylethanolamide or S-777469), which can be administered topically (e.g., dermally or transdermally) or systemically (e.g., orally); or
18) an FAAH inhibitor; or
19) an antidepressant (e.g., an SSRI such as fluvoxamine or paroxetine, or a tricyclic antidepressant such as doxepin or cidoxepin), which can be administered topically (e.g., dermally or transdermally) or systemically (e.g., orally); or
20) NST-141, which can be administered, e.g., topically (e.g., dermally or transdermally); or
21) a moisturizer or emollient (e.g., a moisturizer containing an occlusive such as an oil, or a humectant such as urea); or
22) a counterirritant (e.g., capsaicin) or/and a cooling agent (e.g., a local anesthetic or calamine); or
23) a local anesthetic (e.g., polidocanol), which can be administered, e.g., topically (e.g., dermally or transdermally); or
24) vitamin D or an analog or derivative thereof; or
25) a long-chain polyunsaturated fatty acid (e.g., an n-3[omega-3] fatty acid, such as α-linolenic acid [ALA], eicosapentaenoic acid (EPA) or docosahexaenoic acid [DHA]); or
26) UVB (e.g., narrow-band UVB such as 311-313 nm) phototherapy or UVA (e.g., UVA1) phototherapy with a skin photosensitizer (e.g., psoralen in PUVA); or
27) any combinations thereof.
   41. The method of embodiment 38 or 39, wherein the pruritus is associated with, or/and the medical condition is, psoriasis (e.g., plaque psoriasis), and the one or more additional antipruritic or therapeutic agents are or comprise:
1) in general one or more anti-inflammatory agents that can be administered topically (e.g, dermally or transdermally) or/and systemically (e.g, orally or parenterally); or
2) a topical corticosteroid of moderate or medium potency or a potent or very potent topical corticosteroid; or
3) an immunosuppressant (e.g., alefacept, mycophenolate mofetil, an antimetabolite such as hydroxyurea, an antifolate [e.g., methotrexate] or a purine analog [e.g., azathioprine or thioguanine], a calcineurin inhibitor such as ciclosporin, an mTOR inhibitor such as rapamycin, or a corticosteroid), which can be administered topically (e.g., dermally or transdermally) or systemically (e.g., orally or parenterally); or
4) an inhibitor of a pro-inflammatory cytokine or a receptor therefor (e.g., an inhibitor of TNF-α [e.g., adalimumab, certolizumab pegol, infliximab or etanercept] or an inhibitor of a pro-inflammatory interleukin or a receptor therefor, such as IL-12 [e.g., ustekinumab] or IL-12R, IL-17 [e.g., ixekizumab or secukinumab] or IL-17R [e.g., brodalumab], IL-20 [e.g., the antibody 7E] or IL-20R, IL-22 [e.g., fezakinumab] or IL-22R, or IL-23 [e.g., guselkumab, risankizumab, tildrakizumab or ustekinumab] or IL-23R), which can be administered, e.g., systemically (e.g., orally or parenterally); or
5) an inhibitor of the production of a pro-inflammatory cytokine or a receptor therefor (e.g., an inhibitor of the production of TNF-α [e.g., a PDE4 inhibitor such as apremilast or crisaborole, a p38 MAP kinase inhibitor such as BMS-582949, or a TLR inhibitor {e.g., a TLR7/TLR9 inhibitor such as IMO-3100}], IL-2 [e.g., a PDE4 inhibitor such as apremilast or crisaborole], [e.g., an inhibitor of a TLR such as TLR7 or TLR9], IL-8 [e.g., alefacept], IL-12 [e.g., apilimod], IL-117 [e.g., a PKC inhibitor such as sotrastaurin], or IL-23 [e.g., apilimod or alefacept]), which call be administered topically (e.g., dermally or transdermally) or systemically (e.g., orally or parenterally); or
6) an inhibitor of a pro-inflammatory transcription factor (e.g., an NF-κB inhibitor or a STAT protein inhibitor [e.g., a JAK inhibitor such as tofacitinib]), which can be administered, e.g., systemically (e.g., orally or parenterally); or
7) an inhibitor of NGF or a receptor therefor (e.g., a TrkA inhibitor such as CT327), which can be administered topically (e.g., dermally or transdermally) or systemically (e.g., orally or parenterally); or
8) an inhibitor of CGRP or receptor therefor, which can be administered topically (e.g., dermally or transdermally) or systemically (e.g., orally or parenterally); or
9) an inhibitor of CRH or a receptor therefor, which can be administered topically (e.g., dermally or transdermally) or systemically (e.g., orally or parenterally); or
10) an inhibitor of VIP or a receptor therefor, which can be administered topically (e.g., dermally or transdermally) or systemically (e.g., orally or parenterally); or
11) an inhibitor of somatostatin or a receptor therefor, which can be administered topically (e.g., dermally or transdermally) or systemically (e.g., orally or parenterally); or
12) an antihistamine (e.g., an H₄ antihistamine such as JNJ-7777120 or ZPL-389), which can be administered topically (e.g., dermally or transdermally) or systemically (e.g., orally); or
13) an inhibitor of a mitogen-activated protein kinase (e.g., a p38 MAP kinase inhibitor such as BMS-582949), which can be administered, e.g., systemically (e.g., orally or parenterally); or
14) an inhibitor of the growth o/land proliferation of cells, including skin cells and immune cells (e.g., a retinoid [e.g., acitretin], an NF-κB inhibitor, a STAT protein inhibitor [e.g., a JAK inhibitor such as tofacitinib], a MAP kinase inhibitor [e.g., a p38 MAP kinase inhibitor], an inhibitor of NGF or a receptor therefor [e.g., a TrkA inhibitor such as CT327], or an inhibitor of a proliferation-inducing cytokine or a receptor therefor or the production thereof [e.g., TNF-α, IFN-α, IL-1, IL-2, IL-7, IL-15, IL-17, IL-20, IL-21, IL-22 or IL-23), which can be administered topically (e.g, dermally or transdermally) or systemically (e.g., orally or parenterally), or
15) a retinoid tazarotene or acitretin), which can be administered topically (e.g., dermally or transdermally) or systemically (e.g., orally or parenterally); or
16) an antioxidant (e.g., a poly phenol, a retinoid, or an activator of nuclear factor (erythroid-derived 2)-like 2 [NFE2L2 or Nrf2] [e.g., a fumarate such as dimethyl fumarate]), which can be administered topically (e.g., dermally or transdermally) or systemically (e.g., orally or parenterally); or
17) an anthrone derivative (e.g., dithranol [anthralin]), which can be administered, e.g., topically (e.g., dermally or transdermally); or
18) vitamin D (e.g., vitamin D₂ or vitamin D₃) or an analog or derivative thereof (e.g., calcitriol, calcipotriol or paricalcitol), which can be administered, e.g., topically (e.g., dermally or transdermally); or
19) a counterirritant (e.g., capsaicin) or/and a cooling agent (e.g., a local anesthetic), which can be administered, e.g., topically (e.g., dermally or transdermally); or
20) a moisturizer or emollient (e.g., a moisturizer containing an occlusive such as mineral oil or petroleum jelly, or a humectant such as urea); or
21) coal tar; or
22) UVB (e.g., narrow-band UVB such as 311-313 nm) phototherapy or UVA phototherapy with a skin photosensitizer (e.g., psoralen in PUVA); or
23) laser (e.g., excimer laser) therapy; or
24) any combinations thereof.
   42. The method of embodiment 38 or 39, wherein the pruritus is associated with, or/and the medical condition is, prurigo (e.g., prurigo nodularis), and the one or more additional antipruritic or therapeutic agents are or comprise:
1) a topical corticosteroid (e.g., betamethasone or a derivative thereof) of moderate or medium potency to high or very high potency, or a systemically (e.g., orally or parenterally) administered corticosteroid (e.g., prednisone or a derivative thereof); or
2) a topical immunosuppressant (e.g., a calcineurin inhibitor such as pimecrolimus or tacrolimus), or a systemically (e.g., orally or parenterally) administered immunosuppressant (e.g., an antimetabolite such as an antifolate [e.g., methotrexate] or a purine analog [e.g., azathioprine], or a caleineurin inhibitor such as ciclosporin); or
3) an immunomodulator (e.g., an imide such as thalidomide), which can be administered, e.g., systemically (e.g., orally or parenterally); or
4) an inhibitor of a pro-inflammatory cytokine or a receptor therefor or the production thereof (e.g., an antibody targeting IL-31 or IL-31R such as nernolizumab), or
5) an anti-allergic agent (e.g., tranilast), which can be administered, e.g., systemically (e.g., orally or parenterally); or
6) an antihistamine (e.g., loratadine or cetirizine), which can be administered topically (e.g., dermally or transdermally) or systemically (e.g., orally or parenterally); or
7) an inhibitor of CGRP or receptor therefor, which call be administered topically (e.g., dermally, or transdermally) or systemically (e.g., orally or parenterally); or
8) an inhibitor of NGF or a receptor therefor (e.g., a TrkA inhibitor such as CT327), which can be administered topically (e.g., dermally or transdermally) or systemically (e.g., orally or parenterally); or
9) a mu-opioid receptor antagonist (e.g., naltrexone), which can be administered topically (e.g., dermally or transdermally) or systemically (e.g., orally or parenterally); or
10) a kappa-opioid receptor agonist (e.g., nalfurafine, asimadoline, difelikefalin [CR845] or nalbuphine), which can be administered, e.g., systemically (e.g., orally or parenterally); or
11) a cannabinoid receptor agonist (e.g., palmitoylethanolamide or S-777469), which can be administered topically (e.g., dermally or transdermally) or systemically (e.g., orally); or
12) an anticonvulsant (e.g., gabapentin or pregabalin), which can be administered, e.g., systemically (e.g., orally or parenterally); or
13) an antidepressant (e.g., a tricyclic antidepressant such as amitriptyline, doxepin or cidoxepin, or an SSRI such as fluvoxamine or paroxetine), which can be administered topically (e.g., dermally or transdermally) or systemically (e.g., orally or parenterally); or
14) a local anesthetic (e.g., polidocanol), which can be administered, e.g., topically (e.g., dermally or transdermally); or
15) a moisturizer or emollient; or
16) a counterirritant (e.g., capsaicin) or/and a cooling agent (e.g., a local anesthetic), or a substance that is both a counterirritant and a cooling agent (e.g., camphor or menthol), which can be administered, e.g., topically (e.g., dermally or transdermally); or
17) vitamin D (e.g., vitamin D₃) or an analog or derivative thereof, which can be administered, e.g., topically (e.g., dermally or transdermally); or
18) UVB (e.g., narrow-band UVB such as 311-313 nm) phototherapy or UVA phototherapy with a skin photosensitizer psoralen in PUVA); or
19) any combinations thereof.
   43. The method of embodiment 38 or 39, wherein the pruritus is associated with, or/and the medical condition is, urticaria (e.g., chronic idiopathic urticaria), and the one or more additional antipruritic or therapeutic agents are or comprise:
1) in general one or more, anti-inflammatory agents that can be administered topically (e.g., dermally or transdermally) or/and systemically (e.g., orally or parenterally); or
2) an antihistamine (e.g., a second-generation H₁ antihistamine such as cetirizine, cidoxepin, loratadine or desloratadine, or/and a first-generation H₁ antihistamine such as diphenhydramine, doxepin or hydroxyzine, and optionally an H₂ antihistamine such as cimetidine [e.g., cidoxepin or/and hydroxyzine, or hydroxyzine and cimetidine]), which can be administered topically (e.g., dermally or transdermally) or systemically (e.g., orally or parenterally); or
3) an inhibitor of a leukotriene or a receptor therefor or the production thereof (e.g., a leukotriene receptor antagonist such as montelukast or zafirlukast); or
4) a mast cell stabilizer (e.g., ketotifen); or
5) a glucocorticoid, which can be administered topically (e.g., dermally or transdermally) or systemically (e.g., orally or parenterally); or
6) an inhibitor of a pro-inflammatory cytokine or a receptor therefor or the production thereof (e.g., a TLR9 inhibitor such as hydroxychloroquine); or
7) a myeloperoxidase inhibitor (e.g., dapsone); or
8) an IgE inhibitor (e.g., an anti-IgE antibody such as omalizumab); or
9) a DMARD (e.g., sulfasalazine); or
10) an anti-allergic agent; or
11) an immunosuppressant (e.g., mycophenolate, a calcineurin inhibitor such as cyclosporine or tacrolimus, or an mTOR inhibitor such as rapamycin); or
12) a PAR2 antagonist (e.g., a tetracycline) or a serine protease inhibitor (e.g., camostat or nafamostat); or
13) an MRGPRX2 antagonist; or
14) a moisturizer or emollient; or
15) a counterirritant (e.g., capsaicin) or/and a cooling agent (e.g., a local anesthetic or calamine); or
16) UVB (e.g., narrow-band UVB such as 311-313 nm) phototherapy or UVA phototherapy with a skin photosensitizer (e.g., psoralen in PUVA); or 17) any combinations thereof.
   44. The method of embodiment 38 or 39, wherein the pruritus is associated with, or/and the medical condition is, cutaneous T-cell lymphoma (e.g., mycosis fungoides), and the one or more additional antipruritic or therapeutic agents are or comprise:
1) a mu-opioid receptor antagonist (e.g., naloxone), which can be administered topically (e.g., dermally or transdermally) or systemically (e.g., orally or parenterally); or
2) a corticosteroid, which can be administered topically (e.g., dermally or transdermally) or systemically (e.g., orally or parenterally); or
3) an immunosuppressant (e.g., an antimetabolite such as an antifolate [e.g., methotrexate] or a purine analog [e.g., azathioprine]); or
4) an immune-response modifier (e.g., gardiquimod, imiquimod or resiquimod), which can be administered, e.g., topically (e.g., dermally or transdermally); or
5) an inhibitor of NGF or a receptor therefor (e.g., a TrkA inhibitor such as CT327), which can be administered topically (e.g., dermally or transdermally) or systemically (e.g., orally or parenterally); or
6) an antihistamine (e.g., an SSRI antihistamine); or
7) a serotonin receptor antagonist; or
8) an antidepressant (e.g., an SSRI such as paroxetine or a tetracyclic antidepressant such as mirtazapine or esmirtazapine); or
9) a moisturizer or emollient; or
10) an anti-cancer agent (e.g., a retinoid X receptor agonist such as a retinoid [e.g., bexarotene], or a histone deacetylase inhibitor [e.g., panobinostat, vorinostat or romidepsin]), which can be administered topically (e.g., dermally or transdermally) or systemically (e.g., orally or parenterally); or
11) superficial radiation therapy, which can be local or generalized; or
12) UVB (e.g., narrow- or broad-band UVB) phototherapy or UVA phototherapy with a skin photosensitizer (e.g., psoralen PUVA); or
13) any combinations thereof.
   45. The method of embodiment 38 or 39, wherein the pruritus is associated with, or/and the medical condition is, epidermolysis bullosa (e.g., EB simplex), and the one or more additional antipruritic or therapeutic agents are or comprise:
1) allantoin, which can be administered, e.g., topically (e.g., dermally or transdermally, such as 3-6% allantoin cream in SD-101); or
2) a sulfinyl isothiocyanate (e.g., sulforaphane or raphanin), which can be administered topically (e.g., dermally or transdermally) or systemically (e.g., orally or parenterally); or
3) granulocyte-colony stimulating factor (G-CSF), which can be administered, e.g., systemically (e.g., orally or parenterally); or
4) a corticosteroid, which can be administered topically (e.g., dermally or transdermally) or systemically (e.g., orally or parenterally); or
5) an immunosuppressant for an autoimmune type of EB (e.g., EB acquisita), which can be administered topically (e.g., dermally or transdermally) or systemically (e.g., orally or parenterally); or
6) an antidepressant (e.g., a tricyclic antidepressant such as doxepin or cidoxepin), which can be administered topically (e.g., dermally or transdermally) or systemically (e.g., orally or parenterally); or
7) a moisturizer or emollient (e.g., a moisturizer containing an occlusive such as petroleum jelly); or
8) photopheresis; or
9) any combinations thereof.
   46. The method of embodiment 38 or 39, wherein the pruritus is associated with a burn (e.g., a thermal burn, a second-degree burn or a third-degree burn, or a moderate burn or a major hum), and the one or more additional antipruritic or therapeutic agents are or comprise:
1) an antihistamine (e.g., an H₁ antihistamine such as chlorpheniramine, diphenhydramine, or hydroxyzine which can be administered topically (e.g., dermally or transdermally) or systemically (e.g., orally or parenterally); or
2) an anticonvulsant (e.g., gabapentin which can be administered, e.g., systemically (e.g., orally or parenterally); or
3) a mu-opioid receptor antagonist (e.g., naltrexone), which can be administered topically (e.g., dermally or transdermally) or systemically (e.g., orally or parenterally); or
4) a corticosteroid, which can be administered, e.g., topically (e.g., dermally or transdermally); or
5) a counterirritant (e.g., capsaicin) or/and a cooling agent (e.g., a local anesthetic or calamine), or a substance that is both a counterirritant and a cooling agent (e.g., camphor or menthol), which can be administered, e.g., topically (e.g., dermally or transdermally); or
6) a moisturizer or emollient (e.g., a moisturizer containing a humectant such as honey, an occlusive such as silicone gel, or colloidal oatmeal); or
7) UVB (e.g., narrow-band UVB such as 311-313 nm) phototherapy or UVA phototherapy with a skin photosensitizer (e.g., psoralen in PUVA); or
8) laser therapy; or
9) transcutaneous electrical nerve stimulation; or
10) massage; or
11) any combinations thereof.
   47. The method of embodiment 38 or 39, wherein the pruritus is associated with, or/and the medical condition is, a hepato-biliary disease (e.g., a cholestatic disorder such as cholestasis or primary biliary cirrhosis [PBC]), and the one or more additional antipruritic or therapeutic agents are or comprise:
1) a bile salt-chelating or -sequestering agent (e.g., an ion-exchange resin such as cholestyramine); or
2) a cholesterol absorption-reducing or gallstone-dissolving agent (e.g., ursodeoxycholic acid [ursodiol] or chenodeoxycholic acid); or
3) an agonist of the farnesoid X receptor (FXR, aka bile acid receptor) (e.g., cafestol, chenodeoxycholic acid, obeticholic acid or fexaramine); or
4) an inhibitor of lysophosphatidic acid (LPA) or a receptor therefor or the production thereof (e.g., an autotaxin inhibitor); or
5) a mu-opioid receptor antagonist (e.g., nalmefene, naloxone or naltrexone); or
6) a kappa-opioid receptor agonist (e.g., nalfurafine, asimadoline or difelikefalin [CR845]); or
7) an antidepressant (e.g., an SSRI such as paroxetine or a tetracyclic antidepressant such as mirtazapine); or
8) a serotonin receptor antagonist (e.g, a 5-HT₃ antagonist such as ondansetron or mirtazapine); or
9) an antihistamine; or
10) a glucocorticoid (e.g., prednisone) for, e.g., an inflammatory or autoimmune hepato-biliary disease (e.g., autoimmune hepatitis or PBC); or
11) an immunosuppressant (e.g., an antimetabolite such as a purine analog [e g azathioprine] or a calcineurin inhibitor such as ciclosporin) for, e.g., an inflammatory or autoimmune hepato-biliary disease (e.g., autoimmune hepatitis or PBC); or
12) a copper-chelating agent (e.g., penicillamine) for a hepato-biliary disease in which copper accumulates in the liver (e.g., Wilson's disease or cirrhosis caused thereby); or
13) an antiviral drug for a hepato-biliary disease caused by a virus (e.g., a viral hepatitis such as hepatitis B or C); or
14) S-adenosyl methionine; or
15) rifampicin; or
16) stanozolol; or
17) one or more vitamins (e.g., vitamin A, D, E or K, or any combination or all thereof); or
18) phototherapy (e.g., bright-light therapy, UVB [e.g., narrow-band UVB such as 311-313 nm] phototherapy or U VA phototherapy with a skin photosensitizes [e.g., psoralen in PUVA]); or
19) any combinations thereof.
   48. The method of any one of embodiments 38 to 47, wherein the one or more additional antipruritic or therapeutic agents are administered topically (e.g., dermally or transdermally).
   49. The method of any one of embodiments 38 to 48, wherein the one or more additional antipruritic or therapeutic agents are administered systemically (e.g., orally, intravenously or subcutaneously).
   50. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist selected from aprepitant, fosaprepitant, netupitant, orvepitant, rolapitant, tradipitant, vestipitant, DNK-333, SCH-900978, and pharmaceutically acceptable salts thereof, wherein:

the NK-1 antagonist is not aprepitant for the treatment of pruritus associated with atopic dermatitis or prurigo nodularis;

the NK-1 antagonist is not orvepitant for the treatment of pruritus associated with a burn; and

the NK-1 antagonist is not tradipitant for the treatment of pruritus associated with atopic dermatitis.

51. A method of treating pruritus associated with dermatitis/eczema, psoriasis, prurigo, urticaria, cutaneous T-cell lymphoma, epidermolysis bullosa, a hum or a hepato-biliary disease, comprising administering to a subject in need of treatment a therapeutically effective amount of a neurokinin-1 (NK-1) antagonist and a therapeutically effective amount of an H₄ antihistamine.
52. The method of embodiment 51, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidohenzoic acid, maltooligosaccharides (e.g., maltotetraose and maltopentaose), spantides spantide I and II), AV-608, AV-818, AZD-2624, BIIF 1149 CL, CGP-49823, 0-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.
53. The method of embodiment 51 or 52, wherein the NK-1 antagonist is serlopitant or a pharmaceutically acceptable salt thereof.
54. The method of any one of embodiments 51 to 53, wherein the H₄ antihistamine is selected from clobenpropit, thioperamide, A943931, A987306, JNJ-7777120, VUF-6002, ZPL-389, and pharmaceutically acceptable salts thereof.
55. The method of embodiment 54, wherein the H₄ antihistamine is ZPL-389 or a pharmaceutically acceptable salt thereof.
56. The method of any one of embodiments 51 to 55, wherein the pruritus is associated with dermatitis/eczema (e.g., atopic dermatitis) or psoriasis (e.g., plaque psoriasis).
57. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist and a therapeutically effective amount of a kappa-opioid receptor agonist.
58. The method of embodiment 57, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidohenzoic acid, maltooligosaccharides (e.g., maltotetraose and maltopentaose), spantides spantide I and II), AV-608, AV-818, AZD-2624, BIIF 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212 NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.
59. The method of embodiment 57 or 58, wherein the NK-1 antagonist is serlopitant or a pharmaceutically acceptable salt thereof.
60. The method of any one of embodiments 57 to 59, wherein the kappa-opioid receptor agonist is selected from asimadoline, bremazocine, butorphanol (a mu antagonist and kappa agonist), difelikefalin (CR845), dynorphin, enadoline, ketazocine, nalbuphine (a mu antagonist and kappa agonist), nalfurafine, salvinorin A, 2-methoxymethyl salvinorin B, 2-ethoxymethyl salvinorin B, 2-fluoroethoxymethyl salvinorin B, spiradoline, tifluadom, BRL-52537, FE 200665, GR-89696, HZ-2, ICI-199,441, ICI-204,448, LPK-26, SA-14867, U-50488, U-69,593, and pharmaceutically acceptable salts thereof.
61. The method of embodiment 60, wherein the kappa-opioid receptor agonist is asimadoline, butorphanol, difelikefalin (CR845), nalbuphine or nalfurafine, or a pharmaceutically acceptable salt thereof.
62. The method of any one of embodiments 57 to 61, wherein the pruritus is associated with dermatitis/eczema (e.g., atopic dermatitis), prurigo prurigo nodularis), or a hepato-biliary disease (e.g., a cholestatic disorder such as cholestasis or primary biliary cirrhosis).
63. The method of any one of embodiments 57 to 62, wherein the kappa-opioid receptor agonist is nalbuphine or a pharmaceutically acceptable salt thereof (e.g., Nalbuphine ER), and the pruritus is associated with prurigo (e.g., prurigo nodularis).
64. A method of treating pruritus associated with dermatitis/eczema, psoriasis, prurigo, urticaria, cutaneous T-cell lymphoma (CTCL), epidermolysis bullosa, a burn or a hepato-biliary disease, comprising administering to a subject in need of treatment a therapeutically effective amount of a neurokinin-1 (NK-1) antagonist and a therapeutically effective amount of a mu-opioid receptor antagonist, wherein the NK-1 antagonist is not serlopitant.
65. The method of embodiment 64, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, tradipitant, vestipitant, vofopitant, hydroxyphenyl propamidobenzoic acid, maltooligosaccharides maltotetraose and maltopentaose), spantides (e.g., spantide I and II), AV-608, AV-818, AZD-2624, BIIF 11149 CL, CGP-49823, CJ-17493, CP-96:345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.
66. The method of embodiment 64 or 65, wherein the mu-opioid receptor antagonist is selected from alvimopan, axelopran, bevenopran, butorphanol (a mu antagonist and kappa agonist), cyprodime, eptazocine, levallorphan (lorfan or naloxiphan), methlylnaltrexone, naldemedine, nalmefene, nalbuphine (a mu antagonist and kappa agonist), nalodeine, nalorphine (lethidrone or minim), naloxegol, naloxone, naloxol, naltrexone, naltrexol, samidorphan, SK-1405, and pharmaceutically acceptable salts thereof.
67. The method of embodiment 66, wherein the mu-opioid receptor antagonist is butorphanol, nalmefene, naloxone, naltrexone or SK-1405, or a pharmaceutically acceptable salt thereof.
68. The method of any one of embodiments 64 to 67, wherein the pruritus is associated with dermatitis/eczema (e.g., atopic dermatitis), prurigo (e.g., prurigo nodularis), CTCL (e.g., mycosis fungoides), a burn, or a hepato-biliary disease (e.g., a cholestatic disorder such as cholestasis or primary biliary cirrhosis).
69. A method of treating pruritus associated with dermatitis/eczema, psoriasis, prurigo, urticaria, cutaneous T-cell lymphoma (CTCL), epidermolysis bullosa, a burn or a hepato-biliary disease, comprising administering to a subject in need of treatment a therapeutically effective amount of a neurokinin-1 (NK-1) antagonist and a therapeutically effective amount of an antidepressant, wherein the NK-1 antagonist is not serlopitant.
70. The method of embodiment 69, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIIF 1149 CL, CGP-49823, CJ-17493, CP-96:345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974. ZD-6021, and pharmaceutically acceptable salts thereof.
71. The method of embodiment 69 or 70, wherein the antidepressant is selected from tricyclic antidepressants (e.g., amitriptyline, amitriptylinoxide, amoxapine, dosulepin [dothiepin], doxepin, cidoxepin and melitracen), tetracyclic antidepressants (e.g., amoxapine, maprotiline, mazindol, mianserin, mirtazapine, esmirtazapine and setiptiline), selective serotonin reuptake inhibitors (SSRIs, e.g., citalopram, dapoxetine, escitalopram, fluoxetine, fluvoxamine, paroxetine and sertraline), serotonin-norepinephrine reuptake inhibitors (SNRIs, e.g., bicifadine, doxepin, cidoxepin, duloxetine, milnacipran, levomilnacipran, sibutramine, venlafaxine, desvenlafaxine and SEP-227162), inhibitors of monoamine oxidases (e.g., selective MAO-A inhibitors [e.g., bifemelane, moclobemide, pirlindole {pirazidol} and toloxatone], selective MAO-B inhibitors [e.g., rasagiline and selegiline], and non-selective MAO-A/MAO-B inhibitors [e.g., hydracarbazine, isocarboxazid, nialamide, phenelzine and tranylcypromine]), and pharmaceutically acceptable salts and combinations thereof.
72. The method of embodiment 71, wherein the antidepressant is or comprises amitriptyline, doxepin, cidoxepin, mirtazapine, esmirtazapine, fluvoxamine or paroxetine, or a pharmaceutically acceptable salt or any combination thereof.
73. The method of any one of embodiments 69 to 72, wherein the pruritus is associated with dermatitis/eczema (e.g., atopic dermatitis), prurigo (e.g., prurigo nodularis), CTCL (e.g., mycosis fungoides), epidermolysis bullosa (e.g., epidermolysis bullosa simplex), or a hepato-biliary disease (e.g., a cholestatic disorder such as cholestasis or primary biliary cirrhosis).
74. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist and a therapeutically effective amount of an inhibitor of a pro-inflammatory cytokine or a receptor therefor.
75. The method of embodiment 74, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIIF 1149 CL, CGP-49823, 0-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.
76. The method of embodiment 74 or 75, wherein the NK-1 antagonist is serlopitant or a pharmaceutically acceptable salt thereof.
77. The method of any one of embodiments 74 to 76, wherein the inhibitor of a pro-inflammatory cytokine or a receptor therefor is selected from inhibitors of tumor necrosis factor-alpha (TNF-α) (e.g., adalimumab, certolizumab pegol, golimumab, infliximab, etanercept, bupropion and ART-621), inhibitors of interleukin-2 (IL-2) or receptor therefor (IL-2R) basiliximab and daclizumab), inhibitors of IL-4 or IL-4R (e.g., dupilumab), inhibitors of IL-12 (e.g., briakinumab and ustekinumab) or IL-12R, inhibitors of IL-17 (e.g., ixekizumab and secukinumab) or IL-17R (e.g., brodalumab), inhibitors of IL-22 (e.g., fezakinumab) or IL-22R, inhibitors of IL-23 (e.g., briakinumab, guselkumab, risankizumab, tildrakizumab [SCH-900222], ustekinumab and BI-655066) or IL-23R, inhibitors of IL-31 or IL-31R (e.g., nemolizumab), and pharmaceutically acceptable salts and combinations thereof.
78. The method of any one of embodiments 74 to 77, wherein the pruritus is associated with dermatitis/eczema (e.g., atopic dermatitis), psoriasis (e.g., plaque psoriasis), or prurigo (e.g., prurigo nodularis).
79. The method of any one of embodiments 74 to 78, wherein the inhibitor of a pro-inflammatory cytokine or a receptor therefor is or comprises an inhibitor of IL-2 or IL-2R (e.g., basiliximab or daclizumab), an inhibitor of IL-4 or IL 4R (e.g., dupilumab), or an inhibitor of IL-31 or IL-31R (e.g., nemolizumab), or a pharmaceutically acceptable salt or any combination thereof, and the pruritus is associated with dermatitis/eczema. (e.g., atopic dermatitis).
80. The method of any one of embodiments 74 to 78, wherein the inhibitor of a pro-inflammatory cytokine or a receptor therefor is or comprises a TNF-α inhibitor (e.g., adalimumab, certolizumab pegol, infliximab or etanercept), an inhibitor of IL-12 (e.g., ustekinumab) or IL-12R, an inhibitor of IL-17 (e.g., ixekizumab or secukinumab) or IL-17R (e.g., brodalumab), an inhibitor of IL-22 (e.g., fezakinumab) or IL-22R, or an inhibitor of IL-23 (e.g., guselkumab, risankizumab, tildrakizumab or ustekinumab) or IL-23R, or a pharmaceutically acceptable salt or any combination thereof, and the pruritus is associated with psoriasis (e.g., plaque psoriasis).
81. The method of any one of embodiments 74 to 78, wherein the inhibitor of a pro-inflammatory cytokine or a receptor therefor is or comprises an inhibitor of IL-31 or IL-31R (e.g., nemolizumab or a pharmaceutically acceptable salt thereof), and the pruritus is associated with prurigo (e.g., prurigo nodularis).
82. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist and a therapeutically effective amount of a phosphodiesterase-4 (PDE4) inhibitor, wherein the NK-1 antagonist is not serlopitant for the treatment of pruritus associated with psoriasis.
83. The method of embodiment 82, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides spantide I and II), AV-608, AV-818, AZD-2624, BIIF 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, 8-116031, R-116301, RP-67580, SCH-206272, SCH-188714, SCH-900978, SLV-317, SSR-240600, T-2328, TA-5538, TAK-637, TKA-731, ZD-4974, ZD-6021, and pharmaceutically acceptable salts thereof.
84. The method of embodiment 82 or 83, wherein the NK-1 antagonist is serlopitant or a pharmaceutically acceptable salt thereof.
85. The method of any one of embodiments 82 to 84, wherein the PDE4 inhibitor is selected from apremilast, cilomilast, ibudilast, piclamilast, roflumilast, crisaborole, diazepam, luteolin, mesembrenone, rolipram, AN2728, E6005, and pharmaceutically acceptable salts thereof.
86. The method of embodiment 85, wherein the PDE4 inhibitor is apremilast or crisaborole or a pharmaceutically acceptable salt thereof.
87. The method of any one of embodiments 82 to 86, wherein the pruritus is associated with dermatitis/eczema (e.g., atopic dermatitis) or psoriasis (e.g., plaque psoriasis).
88. The method of any one of embodiments 82 to 87, wherein the PDE4 inhibitor is apremilast or a pharmaceutically acceptable salt thereof, and the pruritus is associated with psoriasis (e.g., plaque psoriasis).
89. A method of treating pruritus associated with a hepato-biliary disease, comprising administering to a subject in need of treatment a therapeutically effective amount of a neurokinin-1 (NK-1) antagonist and a therapeutically effective amount of a farnesoid X receptor (FXR) agonist.
90. The method of embodiment 89, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIIF 1149 CL, CGP-49823, CJ-17493, CP-96345, CP-99994, CP-122721, DNK-333, FR-224, FK-888, GR-205171, GSK-424887, HSP-117, KRP-103, L-703606, L-733060, L-736281, L-759274, L-760735, LY-686017, M516102, MDL-105212, NKP-608, 8-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.
91. The method of embodiment 89 or 90, wherein the NK-1 antagonist is serlopitant or a pharmaceutically acceptable salt thereof.
92. The method of any one of embodiments 89 to 91, wherein FXR agonist is selected from cafestol, chenodeoxycholic acid, obeticholic acid, fexaramine, and pharmaceutically acceptable salts thereof.
93. The method of embodiment 92, wherein the FXR agonist is obeticholic acid or a pharmaceutically acceptable salt thereof.
94. The method of any one of embodiments 89 to 93, wherein the pruritus is associated with a cholestatic disorder (e.g., cholestasis or primary biliary cirrhosis [aka primary biliary cholangitis]).
95. The method of embodiment 94, further comprising administering a cholesterol absorption-reducing or gallstone-dissolving agent ursodeoxycholic acid [ursodiol] or chenodeoxycholic acid).
96. A method of treating pruritus associated with dermatitis/eczema, psoriasis, prurigo, urticaria, cutaneous T-cell lymphoma (CTCL), epidermolysis bullosa, a burn or a hepato-biliary disease, comprising administering to a subject in need of treatment a therapeutically effective amount of a neurokinin-1 (NK-1) antagonist and a therapeutically effective amount of an additional therapeutic agent, wherein:
the additional therapeutic agent is or comprises asimadoline, difelikefalin (CR845), nalbuphine, nalfurafine, SK-1405, S-777469, ZPL-389, CT327, apremilast, crisaborole, EBI-005, dupilumab, nemolizumab, NST-141 or SD-101 or a pharmaceutically acceptable salt or any combination thereof;
the NK-1 antagonist is not serlopitant for use in combination with CT327 to treat pruritus associated with atopic dermatitis, psoriasis or CTCL; and
the NK-1 antagonist is not serlopitant for use in combination with apremilast or crisaborole to treat pruritus associated with psoriasis.
   97. The method of embodiment 96, wherein the NK-1 antagonist is not serlopitant for use in combination with nalbuphine.
   98. The method of embodiment 96, wherein the NK-1 antagonist is not serlopitant for use in combination with SK-1405.
   99. The method of any one of embodiments 96 to 98 wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIIF 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.
   100. The method of embodiment 99, wherein the NK-1 antagonist is serlopitant or a pharmaceutically acceptable salt thereof.
   101. A method of preventing pruritus, comprising administering to a subject a therapeutically effective amount of a neurokinin-1 (NK-1) antagonist prior to development of pruritus.
   102. The method of embodiment 101 wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides (e.g., spantide 1 and II), AV-608, AV-818, AZD-2624, BIIF 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, 8-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.
   103. The method of embodiment 101 or 102, wherein the NK-1 antagonist is serlopitant or a pharmaceutically acceptable salt thereof.
   104. The method of any one of embodiments 101 to 103, wherein the pruritus is acute pruritus.
   105. The method of any one of embodiments 50 to 104, further comprising administering one or more additional antipruritic or therapeutic agents.

### EXAMPLES

The following examples are intended only to illustrate the disclosure. Other procedures, methodologies, techniques, conditions, materials and substances may alternatively be used as appropriate, and other assays and studies may be conducted. All of the inactive pharmaceutical ingredients in the examples below comply with United States Pharmacopeia and The National Formulary requirements and are tested and released according to the monograph for each ingredient specified in the USP/NF compendium.

### Example 1. Preparation of Serlopitant Tablets

The NK-1 antagonist serlopitant can be formulated as a tablet for oral use. Table 1 shows qualitative/quantitative composition of exemplary dosages. Minor variations in the excipient quantities (+/−10%) may occur during the drug development process.

TABLE 1

| Components | Function | % of Composition |
|---|---|---|
| Serlopitant | Active agent | 1-6% |
| Microcrystalline Cellulose | Diluent | 50-60% |
| Mannitol | Diluent | 20-30% |
| Croscarmellose Sodium | Disintegrant | 1-3% |
| Colloidal Silica | Disintegrant | 0.25-0.5% |
| Sodium Sulfate | Surfactant | 5-6% |
| Magnesium Stearate | Lubricant | 0.25-2% |

| | |
|---|---|
| Total Tablet Composition | 100% |

Tablet potencies of 0.25 mg, 1 mg and 5 mg are prepared as a compressed tablet formulation. The tablet manufacturing process is the same for all potencies. The process comprises the following steps: 1) serlopitant, mannitol and sodium lauryl sulfate are blended; 2) the remaining mannitol is added to the blender and mixed; 3) microcrystalline cellulose, croscarmellose sodium and colloidal silica are added to the blender containing the mixture above to complete the mixing, and the blend is de-agglomerated if necessary; 4) the blend is lubricated with magnesium stearate that has been previously screened, if necessary; 5) the lubricated blend is roller-compacted and milled, and then lubricated with magnesium stearate that has been previously screened, if necessary; and 6) the mixture is compressed into tablets of the appropriate weight.

### Example 2. Preparation of Serlopitant Capsules

Serlopitant can also be formulated as liquid-filled capsules. Table 2 shows qualitative/quantitative composition of exemplary dosages. Minor variations in the excipient quantities (+/−10%) may occur during the drug development process,

TABLE 2

| | | Unit Strength | | |
|---|---|---|---|---|
| Components | Function | 0.25 mg | 1 mg | 4 mg |
| Capsule Fill | | | | |
| Serlopitant | Active agent | 0.25 mg | 1 mg | 4 mg |
| Mono- & Di-Glycerides | Solubilizer | 399 mg | 398.6 mg | 395.6 mg |
| Butylated Hydroxyanisole | Antioxidant | 0.40 mg | 0.40 mg | 0.40 mg |
| Capsule Shell | | | | |
| #0 White Opaque Hard Gelatin | Capsule shell | 96 mg** | 96 mg** | 96 mg** |
| Capsule* | | | | |
| Gelatin*** | Banding component | — | — | — |
| Polysorbate 80*** | Banding component | — | — | — |

*Capsules are provided by Capsugel (Morristown, NJ) and contain gelatin and titanium dioxide
**Approximate weight of empty capsule shell
***As needed to seal the capsule shells

The formulation is prepared by dissolving the drug substance in mono- and di-glycerides. Furthermore, 0.1 w t % butylated hydroxyanisole is added as an antioxidant. Initial capsule strengths are dispensed into hard gelatin capsules and sealed by spraying with a 1:1 (wt/wt) water:ethanol solution. Subsequent potencies, including 0.25 mg, 1 mg and 4 mg, are dispensed into hard gelatin capsules and sealed with a band of gelatin/polysorbate 80. Corresponding placebo formulations are prepared in a similar manner, but without the addition of the drug substance and the antioxidant.

The capsule manufacturing process is the same for all potencies. The process comprises the following steps: 1) the mono- and di-glycerides are melted at 40° C., if necessary; 2) the mono- and di-glycerides are added to an appropriately sized, jacketed vessel and mixing is initiated; 3) the butylated hydroxyanisole is added to the mono- and di-glycerides and mixed until dissolved (minimum of 10 min); 4) serlopitant is slowly added to the mixture and mixed until dissolved (visual confirmation); 5) the solution is filled into hard gelatin capsules; 6) the filled capsules are sealed with a mixture of gelatin and polysorbate 80; 7) the sealed capsules are allowed to dry overnight and then the capsules are visually inspected for leaking; 8) the acceptable capsules may be weight-sorted, if necessary; and 9) the finished product is packaged in appropriate containers.

### Example 3. Topical Formulations Containing Serlopitant

Table 3 shows various topical formulations containing serlopitant. The formulations contain Vanicream™ Moisturizing Skin Cream (“VM”), Vanicream™ Lite Lotion (“VLL”) or Aquaphor® Healing Ointment (“AP”, from Eucerin) as the base or carrier. VM and VLL are oil-in-water emulsion and AP has an oil base. A stock solution of free base serlopitant (Compound 1, or “Cpd 1”, in Tables 3 and 4) in ethanol (EtOH) was prepared by dissolving free base serlopitant in ethanol to the maximum extent and then filtering the resulting solution through an Anotop® 25 inorganic filter having a 002 micron pore size. Free base serlopitant has a maximum solubility in ethanol of 64.5 wig EtOH, or 6.45% w/w. To prepare a topical formulation, the stock solution of serlopitant/ethanol was added to a tared tube containing a particular amount of the base until the resulting mixture weighed 25.0 g. The mixture was mixed vigorously for 2 minutes using a vibration stand and then was rotated slowly for 4 days. For the “C” formulations, ethanol containing no serlopitant was added so that the “B” and “C” formulations would contain the same amount of base and ethanol.

TABLE 3

| | Lot | | Cpd 1/EtOH | Blank | % | % |
|---|---|---|---|---|---|---|
| | Size | Base | Stock | EtOH | Cpd 1 | EtOH |
| Mixture | (g) | (g) | Soln (g) | (g) | (w/w) | (w/w) |
| VM-A | 25.0 | 23.06 | 1.94 | 0.0 | 0.5 | 7.8 |
| VM-B | 25.0 | 21.12 | 3.88 | 0.0 | 1.0 | 15.5 |
| VM-C | 25.0 | 21.12 | 1.94 | 1.94 | 0.5 | 15.5 |
| VLL-A | 25.0 | 23.06 | 1.94 | 0.0 | 0.5 | 7.8 |
| VLL-B | 25.0 | 21.12 | 3.88 | 0.0 | 1.0 | 15.5 |
| VLL-C | 25.0 | 21.12 | 1.94 | 1.94 | 0.5 | 15.5 |
| AP-A | 25.0 | 23.06 | 1.94 | 0.0 | 0.5 | 7.8 |
| AP-B | 25.0 | 21.12 | 3.88 | 0.0 | 1.0 | 15.5 |
| AP-C | 25.0 | 21.12 | 1.94 | 1.94 | 0.5 | 15.5 |

AP was determined to be an unsuitable base for an ethanol solution containing serlopitant because of ethanol insolubility in that base. The VM base appeared stable/unchanged under 15× microscopic magnification after 4 days of mixing with 15.5% ethanol. The VLL base showed some aggregation of lamellar structures under 15× microscopic magnification after 4 days of mixing with 15.5% ethanol, but the overall change to the base appeared minor. The VM and VLL, formulations can be tested, e.g., for the skin permeation of serlopitant.

### Example 4. In Vitro Skin Permeation of Serlopitant in Topical Formulations

Topical formulations A-D used in the in vitro skin permeation studies are shown in Table 4. The bases “VM” and “VLL” of formulations A-D are described in Example 3. Formulations A-D were prepared according to the procedures described in Example 3.

TABLE 4

| | Final | | Cpd 1/EtOH | Blank | % | % |
|---|---|---|---|---|---|---|
| Formul'n | Mass | Base | Stock | EtOH | Cpd 1 | EtOH |
| (Base) | (g) | (g) | Soln (g) | (g) | (w/w) | (w/w) |
| A (VM) | 25.28 | 21.27 | 0.0 | 4.01 | 0.0 | 15.9 |
| B (VLL) | 25.12 | 21.19 | 3.93 | 0.0 | 1.0 | 15.6 |
| C (VM) | 13.80 | 11.63 | 2.17 | 0.0 | 1.0 | 15.7 |
| D (VLL) | 25.02 | 21.15 | 0.0 | 3.87 | 0.0 | 15.5 |

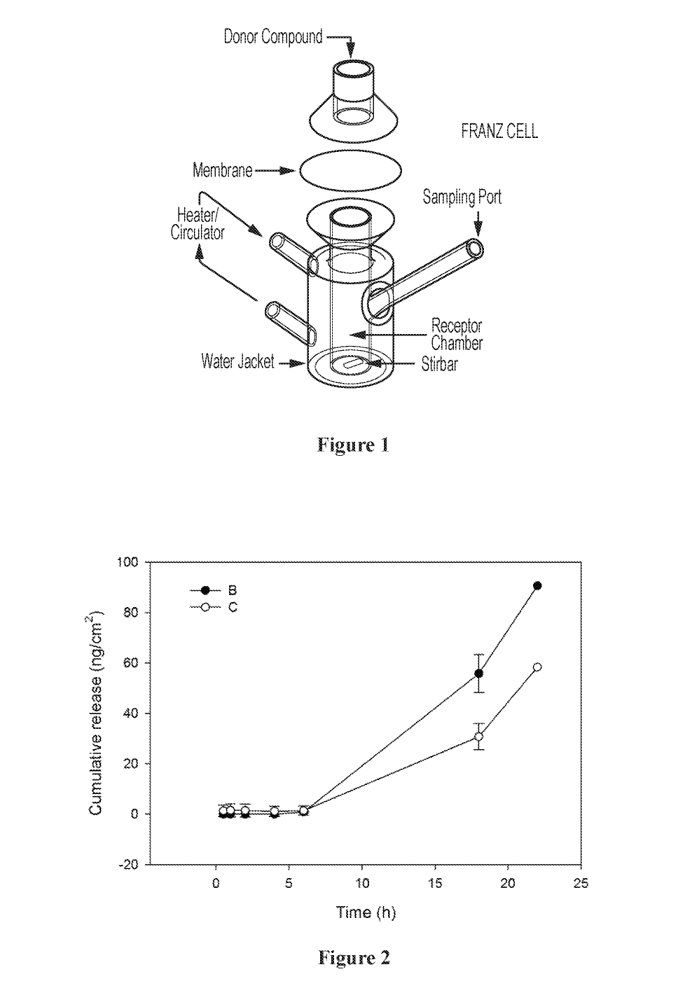

In vitro skin permeation of serlopitant in topical formulations A-D was evaluated using a Franz diffusion cell. FIG. 1 illustrates a Franz diffusion cell. A Franz diffusion cell having a circular permeation area of 4.15 cm² and a receptor chamber volume of 19 mL was set up with a thermo-regulated outer water jacket to maintain the temperature at 37° C. The receptor chamber was filled with 19 mL 1×PBS (pH 7.5) containing 10% ethanol and 1% Tween® 80. Solubility test indicated that serlopitant remained soluble at concentrations of 0.5, 5 and 50 ug/mL, in this solution after 1 hour of incubation at 37° C. The solubility of serlopitant decreased significantly if Tween® 80 was not used and decreased slightly if ethanol was not used.

Human skin was pre-treated to remove all subcutaneous fat and was cleaned with 70% ethanol before use. The skin was visually inspected to ensure that it was free of any surface irregularity or small holes and was equally divided into four pieces. The skin was then mounted onto the receptor chamber with the stratum corneum side facing up. About 100 mg of topical formulation A, B, C or D was applied to the skin (actual weight: A, 103.8 mg; B, 101.3 mg, C, 103.2 mg; and D, 10:3.8 mg), which was then covered with parafilm to avoid evaporation.

About 0.5 ml, of solution was withdrawn through the sampling port of the Franz diffusion cell at 0.5, 1, 2, 4, 6, 18 and 22 hours. The receptor chamber was replenished with equal volume of fresh diffusion buffer after each sampling. At the end of the experiment (after 22 hours of incubation), the skin was wiped clean with methanol, and the formulation-treated area was weighed and frozen for cry ° sectioning.

All samples were processed by solid-phase extraction (SPE) before LC-MS/MS analysis. Briefly, a Strata-X 33 urn Polymeric Reverse-Phase column with 30 mg sorbent mass/1 mL volume (Phenomenex) was conditioned with 1 nit, of methanol and equilibrated with 1 mL of water. 300 AIL of sample was loaded to the column followed by a wash with 1 mL of 30% methanol. Serlopitant was eluted with 2% formic acid in acetonitrile. The sample then was concentrated by blow drying with nitrogen and re-suspended in 50 uL, of 50% methanol. A working standard was first generated by spiking the diffusion buffer with known concentrations of serlopitant, which was then processed using the same SPE method. A sensitivity of 0.1 ng/mL, was achieved. Serlopitant concentrations in samples resulting from formulations A-D were determined by comparison to the standard. Serlopitant was not detected in samples resulting from topical formulations A and D, as expected. FIG. 2 shows the cumulative release of serlopitant from topical formulations B and C into the receptor chamber at 0.5, 1, 2, 4, 6, 18 and 22 hours. After an initial lag, serlopitant was detected by LC-MS/MS in the receptor chamber at 6 hours. FIG. 2 indicates that topical formulation B resulted in greater penetration of serlopitant through the skin than topical formulation C in this in vitro study.

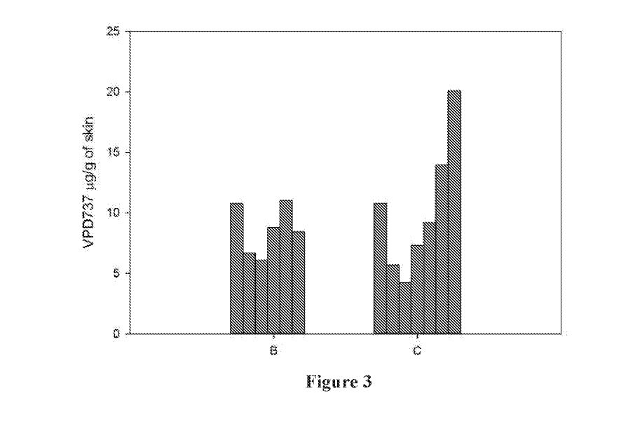

The amount of serlopitant retained in the skin was determined at the end of the experiment. The skin was wiped and washed with methanol. The formulation-treated area was cut into horizontal sections of 25 um using a cryostat. Every 10 sections were pooled, placed in Eppendorf tubes, weighed and digested with twice the volume of 1 mg/mL liberase at 37° C. for 1 hour. Digested skin sections were further homogenized with a probe sonicator. To 25 uL of the skin homogenate were added 25 uL, of 50% methanol and 100 uL of acetonitrile/methanol to extract serlopitant. For spiked standards, 25 uL of a solution of serlopitant in 50% methanol (from 5 ng/mL to 5000 ng/mL) was added to 25 uL of blank skin homogenate followed by 100 uL of acetonitrile/methanol. Extracted serlopitant was quantified by LC-MS/MS. FIG. 3 shows the amount of serlopitant (called “VPD737” in FIG. 3) retained in the skin at the end of the experiment. Each bar represents ug of serlopitant/g of skin in 250 um skin layers. For each of topical formulations B and C, the bars from left to right represent the amount of serlopitant retained in skin layers from the stratum corneum to the dermis.

### Example 5. Representative Topical Formulations Containing an NK-1 Antagonist

Table 5 provides non-limiting examples of topical formulations that can be prepared with an NK-1 antagonist (e.g., serlopitant) or a salt, solvate, hydrate, clathrate, polymorph, prodrug or metabolite thereof, and optionally an additional antipruritic or therapeutic agent.

TABLE 5

| Dosage | |
|---|---|
| Form | Ingredients in Addition to NK-1 Antagonist (e.g., Serlopitant) |
| cream | sorbitol, cetyl alcohol, isopropyl myristate, glyceryl stearate, PEG-100 stearate, |
| | petrolatum, benzyl alcohol, titanium dioxide and water |
| cream | propylene glycol, cetostearyl alcohol, Cremophor ® A6, Cremophor ® A25, liquid |
| | paraffin, parabens and water |
| cream | glycerol, sorbitol, isopropyl palmitate, emulsifying wax, benzyl alcohol, a pH adjuster |
| | (e.g., NaOH or lactic acid), and water |
| cream | glycerol, stearic acid, glyceryl monostearate, triethanolamine, parabens and water |
| cream | propylene glycol, cetostearyl alcohol, mineral oil, white petrolatum, ceteareth-30, |
| | chlorocresol, sodium phosphate monobasic, phosphoric acid, water, and optionally NaOH |
| cream | glycerol, cetostearyl alcohol, mineral oil, petrolatum, ceteth-20, diazolidinyl urea, |
| | dichlorobenzyl alcohol, edetic acid (EDTA) or disodium edetate, dibasic sodium |
| | phosphate and water |
| cream | propylene glycol, steamy alcohol, white petrolatum, polysorbate 60, parabens, and |
| | optionally water |
| cream | propylene glycol, stearyl alcohol, cetyl alcohol, oleyl alcohol, mono-, di- or/and tri- |
| | glycerides, sodium cetostearyl sulphate, benzyl alcohol, citric acid, a pH adjuster (e.g., |
| | NaOH or lactic acid), and water |
| cream | hexylene glycol, stearyl alcohol, propylene glycol stearate, white wax, white petrolatum, |
| | aluminum starch octenylsuccinate, ceteareth-20, titanium dioxide, phosphoric acid and |
| | water |
| cream | propylene glycol, sorbitol, glyceryl monoisostearate, polyglyceryl-3 oleate, mineral oil, |
| | microcrystalline wax, colloidal silicon dioxide, parabens, EDTA or disodium edetate, and |
| | water |
| cream | propylene glycol, stearic acid, isopropyl palmitate, emulsifying wax, beeswax, polysorbate |
| | 60, an antioxidant (e.g., propyl gallate), a preservative (e.g., sorbic acid or/and K⁺ |
| | sorbate), a pH adjuster (e.g., NaOH or/and citric acid), and water |
| cream | cetostearyl alcohol, lanolin alcohols, isopropyl my ristate, aluminum stearate, magnesium |
| | stearate, mineral oil, white petrolatum, water, and optionally disodium edetate or/and |
| | lactic acid |
| cream | propylene glycol, cetostearyl alcohol, white soft paraffin, liquid paraffin, lanolin, |
| | simethicone M30, Tween ® 60, parabens and water |
| cream | cetostearyl alcohol, mineral oil, white petrolatum, ceteth-20, parabens, citric acid, sodium |
| | citrate, and water |
| cream | propylene glycol, cetostearyl alcohol, polyoxyl 20 cetosteatyl ether, mineral oil (liquid |
| | paraffin), petrolatum (white soft paraffin), chlorocresol, parabens, sodium phosphate |
| | monobasic, and water |
| cream | propylene glycol, cetostearyl alcohol, stearic acid, cetyl palmitate, sorbitan monostearate, |
| | mineral oil, polysorbate 60, benzyl alcohol and water |
| ointment | hexylene glycol, propylene glycol stearate, white wax, white petrolatum, phosphoric acid |
| | and water |
| ointment | propylene glycol, mineral oil, petrolatum, steareth-2, tocopherol, EDTA or disodium |
| | edetate, dibasic sodium phosphate and water |
| ointment | propylene glycol, fatty alcohol citrate, fatty acid pentaerythritol ester, sorbitan |
| | sesquioleate, white petrolatum, beeswax, aluminum stearate, butylated hydroxyanisole |
| | (BHA), citric acid, and optionally water |
| ointment | an alcohol (e.g., ethanol or/and propylene glycol), polyethylene or white petrolatum, |
| | mineral oil, and optionally water |
| gel | ethanol, carbomer 934P, triethanolamine and water |
| gel | glycerol, carbomer 940, poloxamer, dimethicone, disodium lauryl sulfosuccinate, silicon |
| | dioxide, a preservative (e.g., benzoyl peroxide or/and methyl paraben), EDTA or disodium |
| | edetate, a pH adjuster (e.g., NaOH or lactic acid), and water |
| gel | glycerol, hydroxy-beta-cyclodextrin, hydroxyethyl cellulose, parabens, EDTA or |
| | disodium edetate, and water |
| gel | propylene glycol, polyacrylic acid, medium-chain triglycerides, lecithin, polysorbate 80, a |
| | preservative (e.g., benzoic acid), EDTA or disodium edetate, a pH adjuster (e.g., NaOH or |
| | lactic acid), and water |
| gel | ethanol, isopropyl myristate, carbomer 940, triethanolamine, docusate sodium, EDTA or |
| | disodium edetate, and water |
| gel | propylene glycol, Carbopol ® 941, PEG 400, methyl paraben, a pH adjuster (e.g., NaOH |
| | or lactic acid), and water |
| gel | propylene glycol, PEG 400, carbomer 934P, allantoin, methyl paraben, a pH adjuster (e.g., |
| | NaOH or lactic acid), and water |
| gel | an alcohol (e.g., ethanol or/and propylene glycol), carbomer, dioctyl sodium |
| | sulfosuccinate, a preservative (e.g., benzoyl peroxide), a pH adjuster (e.g., NaOH or lactic |
| | acid), and water |
| gel | glycerol, propylene glycol, aloe vera gel, diazolidinyl urea, capryl/capramidopropyl |
| | betaine, parabens, citric acid, sodium citrate, and water |
| gel | ethanol, hydroxypropyl cellulose and water |
| lotion | glycerol, stearyl alcohol, glyceryl stearate, PEG-100 stearate, PEG 400, carbomer 941, |
| | cyclomethicone, light mineral oil, steareth-21, benzyl alcohol, sorbic acid or potassium |
| | sorbate, a pH adjuster (e.g., NaOH or lactic acid), and water |
| lotion | isopropanol, propylene glycol, hydroxypropyl cellulose, sodium phosphate monobasic, |
| | phosphoric acid and water |
| lotion | propylene glycol, cetyl alcohol, stearyl alcohol, glyceryl stearate, sorbitan monostearate, |
| | light mineral oil, sodium lauryl sulfate, parabens, EDTA or disodium edetate, water, and |
| | optionally a pH adjuster (e.g., NaOH or citric acid) |
| lotion | glycerol, cetostearyl alcohol, isostearyl alcohol, stearic acid, glycerl stearate, sodium |
| | lauroyl sarcosinate, methyl paraben and water |
| suppository | an alcohol (e.g., ethanol or/and propylene glycol) and glycerides of saturated fatty acids |
| suppository | 95% ethanol and Suppocire ® AM (glyceride base containing saturated C₈-C₈ triglyceride |
| | fatty acids) |
| pledget | isopropanol, propylene glycol and water |
| foam | ethanol, propylene glycol, cetyl alcohol, stearyl alcohol, polysorbate 60, KOH and water, |
| | and pressurized with a propane/butane propellant |
| spray | ethanol, undecylenic acid, isopropyl myristate, sodium lauryl sulfate, and water |
| (dermal) | |
| spray | glycerol, lactose, cetostearyl alcohol, mineral oil, ceteth-20 phosphate, dicetyl phosphate, |
| (dermal) | urea, potassium phosphate monobasic, parabens, a pH adjuster (e.g., NaOH or lactic acid), |
| | and water |
| spray | microcrystalline cellulose, carboxymethyl cellulose sodium, dextrose, polysorbate 80, |
| (nasal) | disodium edetate, potassium sorbate, a pH adjuster (e.g., HCl), water, and optionally an |
| | alcohol (e.g., ethanol) |
| spray | microcrystalline cellulose, carboxymethyl cellulose sodium, dextrose, polysorbate 80, |
| (nasal) | benzalkonium chloride, phenylethyl alcohol, water, and optionally an alcohol (e.g., |
| | ethanol) |
| spray | hypromellose, benzalkonium chloride, NaCl, EDTA, citric acid, sodium phosphate |
| (nasal) | dibasic, water, and optionally an alcohol (e.g., ethanol) |

### Example 6. Clinical Study of Serlopitant for Chronic Pruritus

A well-controlled human clinical trial assessing the efficacy of serlopitant in the treatment of chronic pruritus was approved by an Institutional Review Board and was conducted in accordance with the International Conference on Harmonisation (ICH) Guidelines for Good Clinical Practices, the U.S. Code of Federal Regulations, the Health Insurance Portability and Accountability Act (HIPAA), and any local regulatory requirements. The study was a Phase II randomized, double-blind, parallel-group, placebo-controlled, multicenter trial designed to evaluate the efficacy and safety of serlopitant versus placebo in subjects with chronic pruritus. The study subject population was adult males and females 18 to 65 years old who had pruritus of at least 6-week duration which was unresponsive or inadequately responsive to current therapies such as topical steroids or oral antihistamines, and who had a baseline Visual Analog Scale (VAS) pruritus score of at least 7 on a 10-point scale.

Subjects were randomized to receive a 0.25 mg, 1 mg or 5 mg tablet of serlopitant or a matching placebo tablet. Subjects took one tablet of serlopitant or placebo once daily by mouth for a total of 6 weeks following a loading dose of 3 tablets on the first day of treatment. The maximum study duration for each subject was about 12 weeks and included a screening period of up to 2 weeks, a treatment period of 6 weeks, and a follow-up period of 4 weeks. The screening period was extended up to 44 days if a washout period from any prohibited medications was required. The study parameters are summarized in Table 6.

TABLE 6

| | |
|---|---|
| Study Title: | A Randomized, Double-Blind, Parallel-Group, Placebo- |
| | Controlled Study of Serlopitant in Subjects with Chronic |
| | Pruritus |
| Development Phase: | Phase II |
| Study Objectives: | Evaluate the efficacy and safety of serlopitant in subjects with |
| | chronic pruritus |
| Study Design: | Randomized, double-blind, parallel-group, placebo-controlled |
| Sample Size: | 256 subjects took by random assignment once-daily doses of |
| | 0.25 mg (n = 64), 1 mg (n = 65) or 5 mg (n = 64) of serlopitant |
| | or placebo (n = 63) for 6 weeks |
| Study Population: | Men and women 18 to 65 years old who had pruritus of at least |
| | 6-week duration which was unresponsive or inadequately |
| | responsive to current therapies, and who had a Visual Analog |
| | Scale (VAS) pruritus score ≥7 on a 10-point scale at baseline |
| | and on at least two of the last three available entries in their |
| | electronic diary (eDiary) provided at screening |
| | Subjects could have chronic pruritus associated with an |
| | inflammatory skin disease or one independent of a primary skin |
| | disease. |
| | Subjects could not have pruritus due to urticaria, a drug allergy |
| | or an infection, or pruritus of a neuropathic or psychogenic |
| | etiology. |
| | Subjects could not have chronic renal or liver disease. |
| | Subjects could not have a skin malignancy, or a current |
| | malignancy or a blood cell dyscrasia that could result in |
| | systemic chronic pruritus. |
| | Subjects could not take a drug known to cause pruritus. |
| Investigational Product: | Oral daily tablets of serlopitant |
| Dosage and Frequency | One 0.25 mg, 1 mg or 5 mg tablet of serlopitant once daily by |
| | mouth at bedtime for 6 weeks after a loading dose of 3 tablets |
| | on Day 1 |
| Reference Product: | None |
| Control Product: | One tablet of matching placebo once daily by mouth at bedtime |
| | for 6 weeks following a loading dose of 3 tablets on Day 1 |
| Efficacy Evaluation Criteria: | Efficacy was assessed by subject self-ratings of pruritus |
| | severity recorded twice daily using a 10-point VAS scale and a |
| | 10-point Numerical Rating Scale (NRS) provided in a subject |
| | eDiary. |
| | The primary efficacy endpoint was the percent change from |
| | Baseline/Day 1 in mean VAS pruritus score, comparing each |
| | dose group of serlopitant to placebo. |
| | Secondary efficacy endpoints included analyses of the NRS |
| | score, Dermatology Life Quality Index (DLQI), Pittsburgh |
| | Sleep Symptom Questionnaire-Insomnia/Pittsburgh Sleeping |
| | Quality Index (PSSQ_I), Subject Global Assessment (SGA), |
| | and Physician Global Assessment (PGA). |
| Safety Evaluation Criteria: | Safety was assessed by adverse events, serious adverse events, |
| | electrocardiograms, vital signs, abbreviated physical |
| | examinations, and blood and urine laboratory tests. |
| Statistic Methods: | Statistical analysis of the primary efficacy endpoint was done |
| | using a repeated measures linear mixed effects model (“mixed |
| | effects model”). The model provided pairwise estimates of |
| | treatment effect vs. placebo with the associated confidence |
| | interval. Estimates of treatment effect on VAS were prepared |
| | using pairwise estimates of differences between each |
| | serlopitant dose and placebo with the associated confidence |
| | interval and p-value for each pairwise two-sided test of the null |
| | hypothesis serlopitant dose vs. placebo. |
| Study Sites: | Multiple sites in the United States |

Table 7 shows the least squares mean percent change from Baseline/Day 1 in average VAS pruritus score in subjects with chronic pruritus who took orally placebo or 0.25 mg, 1 mg or 5 mg of serlopitant once daily for 6 weeks. Compared to placebo, a once-daily 1 mg dose and a once-daily 5 mg dose of serlopitant provided statistically significant improvement in relief of itch at Weeks 4, 5 and 6 in the VAS score (the primary efficacy endpoint; Table 7), as well as in the NRS score (a secondary efficacy endpoint; data not shown). In addition, a once-daily 1 mg dose and a once-daily 5 mg dose of serlopitant resulted in a 4-point responder rate (the proportion of subjects achieving ≥4-point improvement on a 10-point scale) of 42% and 53%, respectively, in the average VAS itch score at Week 6 compared to a 4-point responder rate of 26% for placebo at Week 6. All three doses of serlopitant were well tolerated and exhibited an excellent safety profile, with the most common treatment-emergent adverse events being diarrhea, somnolence and headache in the low single-digit percent, and all adverse events being of mild or moderate intensity.

TABLE 7
Least squares mean % change from baseline in average
VAS itch score

| | | Serlopitant | Serlopitant | Serlopitant |
|---|---|---|---|---|
| | Placebo | 0.25 mg | 1 mg | 5 mg |
| Time Point | (n = 63) | (n = 64) | (n = 65) | (n = 64) |
| Week 1 | −4.1 ± 3.9 | −9.1 ± 3.9 | −11.8 ± 3.9 | −12.0 ± 3.9 |
| Week 2 | −12.1 ± 4.0 | −14.9 ± 4.0 | −22.0 ± 4.0 | −20.3 ± 4.0 |
| Week 3 | −18.7 ± 4.0 | −21.4 ± 4.0 | −30.2 ± 4.0 | −28.8 ± 4.0 |
| Week 4 | −21.6 ± 4.0 | −29.0 ± 4.0 | −33.1 ± 4.0* | −34.2 ± 4.0* |
| Week 5 | −25.8 ± 4.1 | −32.4 ± 4.0 | −38.0 ± 4.1* | −37.3 ± 4.0* |
| Week 6 | −28.3 ± 4.1 | −34.1 ± 4.1 | −41.4 ± 4.0* | −42.5 ± 4.1* |

*p < 0.05 vs placebo

### Example 7. Clinical Study of Serlopitant for Chronic Pruritus in Prurigo Nodularis

A well-controlled human clinical trial assessing the efficacy of serlopitant in the treatment of pruritus associated with prurigo nodularis (PN) was approved by an Institutional Review Board and was conducted in accordance with the ICH Guidelines for Good Clinical Practices. German regulations on recordkeeping of subject information, and any local regulatory requirements. The study was a Phase II randomized, double-blind, placebo-controlled, multicenter trial designed to evaluate the efficacy and safety of serlopitant versus placebo in subjects with PN. The study subject population was adult males and females 18 to 80 years of age who had both PN (lesions on both arms, both legs or/and the trunk of the body) and pruritus of more than 6-week duration which were unresponsive or inadequately responsive to topical glucocorticoid or oral antihistamine therapies, and who had a Visual Analog Scale (VAS) pruritus score of at least 70 on a 0 to 100 mm scale within 72 hours of baseline. The subjects had chronic pruritus due to PN.

Subjects were randomized to receive either a 5-mg tablet of serlopitant or a matching placebo tablet. Subjects took a tablet of serlopitant or placebo once daily by mouth for 8 weeks following a loading dose of 3 tablets on the first day of treatment. The maximum study duration for each subject was about 14 weeks and included a screening period of up to 4 weeks, a treatment period of 8 weeks, and a follow-up period of 2 weeks. The study parameters are summarized in Table 8.

TABLE 8

| | |
|---|---|
| Study Title: | A Randomized, Double-Blind, Placebo-Controlled Study of |
| | Serlopitant in Subjects with Chronic Pruritus and Prurigo |
| | Nodularis |
| Development Phase: | Phase II |
| Study Objectives: | Evaluate the efficacy and safety of serlopitant in subjects with |
| | chronic pruritus and prurigo nodularis |
| Study Design: | Randomized, double-blind, placebo-controlled |
| Sample Size: | 127 subjects took by random assignment 5 mg of serlopitant (n = |
| | 64) or placebo (n = 63) once daily for 8 weeks |
| Study Population: | The subjects were men and women 18 to 80 years old who had |
| | both prurigo nodularis (PN) and pruritus of more than 6-week |
| | duration which were unresponsive or inadequately responsive |
| | to topical glucocorticoid or oral antihistamine therapies, and |
| | who had a Visual Analog Scale (VAS) pruritus score ≥70 on a |
| | 0 to 100 mm scale within 72 hours of baseline. They had |
| | chronic pruritus due to PN. |
| | Subjects could not have suspected drug-induced PN or pruritus. |
| Investigational Product: | Oral daily tablet of serlopitant |
| Dosage and Frequency | Loading dose of three 5-mg tablets of serlopitant or matching |
| | placebo on Day 1, followed by one 5-mg tablet of serlopitant or |
| | matching placebo once daily by mouth at bedtime for 8 weeks |
| Reference Product: | None |
| Control Product: | Matching placebo |
| Efficacy Evaluation Criteria: | Efficacy was assessed by subject self-ratings of pruritus |
| | intensity recorded once daily in a subject's electronic diary |
| | (eDiary). |
| | The primary efficacy endpoint was change from Baseline in the |
| | average itch VAS score. Subjects reported their average itch |
| | over the last 24 hours on a 10 cm VAS. Results at Week 4 and |
| | Week 8 were the primary time points. |
| | Secondary efficacy endpoints in pruritus and PN assessment |
| | included comparisons between serlopitant and placebo of: |
| | mean change from Baseline in Verbal Rating Scale (VRS), |
| | worst itch VAS (worst itch over the past 24 hours), |
| | Numerical Rating Scale (NRS), global and dynamic scores, |
| | Dermatology Life Quality Index (DLQI), Pruritus-Specific |
| | Quality of Life (ItchyQoL), Patient Benefit Index for Patients |
| | with Pruritus (PBI-P), and Patient and investigator Global |
| | Assessments (PGA and IGA) scores and results; |
| | mean change from Baseline in PN skin lesions as measured |
| | by the Prurigo Activity Score (PAS); |
| | time course of changes in VRS, worst itch VAS and NRS |
| | pruritus scores; and |
| | percentage of subjects requiring rescue therapy with |
| | loratadine or cetirizine. |
| Safety Evaluation Criteria: | Safety was assessed by adverse events, serious adverse events, |
| | electrocardiograms, vital signs, abbreviated physical |
| | examinations, and blood and urine laboratory tests. |
| Statistical Methods: | The primary efficacy endpoint was analyzed using repeated |
| | measures for average VAS score. The model included change |
| | from baseline as the response variable, and baseline VAS score, |
| | visit, pooled site, treatment and visit by treatment as the |
| | independent variables. Visit was included as a categorical |
| | variable. The model used an unstructured covariance matrix. |
| | The estimated treatment difference at Weeks 2, 4, and 8 was |
| | summarized and a p-value for these comparisons was provided. |
| | The Weeks 4 and 8 tests were considered primary, so there |
| | were two primary comparisons (one for each visit). No |
| | multiplicity adjustment was used. |
| | The secondary efficacy endpoints were summarized with |
| | descriptive statistics, which included estimates within the |
| | treatment group (e.g., mean results for serlopitant) and for |
| | selected endpoints included estimates of the treatment effect, |
| | 95% confidence intervals (Wilson for binary data and Wald for |
| | continuous data), and statistical testing (t-tests. Cochran- |
| | Mantel-Haenszel tests or repeated measures). |
| Study Sites: | Multiple sites in Germany |

Regarding the primary efficacy endpoint, Table 9 shows the mean difference in change of the average itch VAS score from Baseline at Weeks 2, 4, and 8 between subjects with chronic pruritus due to prurigo nodularis who took orally 5 mg of serlopitant or placebo once daily for 8 weeks. At Baseline, the average itch VAS score (average itch over the past 24 hours) for the serlopitant group and the placebo group was 7.88 and 7.92, respectively. Compared to placebo, a once-daily 5 mg dose of serlopitant resulted in a statistically significant decrease (a statistically significantly greater decrease) in the average itch VAS score from Baseline at Weeks 2, 4, and 8. Furthermore, a once-daily 5 mg dose of serlopitant led to a 4-point responder rate (the proportion of subjects achieving ≥4-point improvement on a 10-point scale) of 54% with respect to the average itch VAS score at Week 8 compared to 25% for placebo.

TABLE 9
Mean difference between 5 mg serlopitant and placebo in
change of average itch VAS score from baseline by repeated
measures analysis

| | Mean Difference | (95% Confidence | |
|---|---|---|---|
| Time Point | Serlopitant − Placebo (SE) | Interval) | p-value |
| Week 2 | −0.9 (0.34) | (−1.5, −0.2) | 0.0111 |
| Week 4 | −1.0 (0.43) | (−1.8, −0.1) | 0.0248 |
| Week 8 | −1.7 (0.47) | (−2.6, −0.7) | 0.0005 |

SE = standard error

A once-daily 5 mg dose of serlopitant also demonstrated efficacy in secondary endpoints compared to placebo in subjects with chronic pruritus due to PN. First, there was a greater proportion of subjects reporting “no/mild pruritus” on the VRS, and improvement in pruritus on the PGA, at Week 8 in the serlopitant group (54.4% and 82.5%, respectively) than in the placebo group (28.9% and 54.3%, respectively). Second, serlopitant provided a statistically significantly, greater improvement in the worst itch VAS score from Baseline to Week 8 than placebo (p=0.0024). Third, serlopitant provided a statistically significantly greater decrease in the average itch NRS score from Baseline to Week 8 than placebo (p=0.0069). Fourth, a once-daily 5 mg dose of serlopitant resulted in a 4-point responder rate of 47% with respect to the worst itch NRS score at Week 8 compared to 26% for placebo. Fifth, serlopitant provided greater improvement in pruritus on the IGA than placebo.

Serlopitant was well tolerated and safe in the study, and no significant safety signal was detected. Treatment-emergent adverse events were generally mild or moderate. The most common adverse events were nasopharyngitis (17%) and diarrhea (11%).

### Example 8. Clinical Study of Serlopitant for Chronic Pruritus in Atopic Dermatitis

A well-controlled human clinical trial assessing the efficacy of serlopitant in the treatment of pruritus associated with atopic dermatitis (AD) is conducted in accordance with the ICH Guidelines for Good Clinical Practices, the U.S. Code of Federal Regulations, HIPAA and any local regulatory requirements. The study is a Phase II randomized, double-blind, placebo-controlled, multicenter trial designed to evaluate the efficacy, tolerability and safety of serlopitant versus placebo in subjects with a history of AD. The study subject population includes adult males and females 18-65 years of age. The subjects have a diagnosis of active AD or a documented past diagnosis of AD and have pruritus of at least 6-week duration despite treatment with standard-of-care antipruritic therapies such as oral H₁ antihistamines, topical corticosteroids and emollients.

Subjects are randomized to receive either a 5-mg tablet of serlopitant or a matching placebo tablet. Subjects take a tablet of serlopitant or placebo once daily by mouth for a total of 6 weeks following a loading dose of 3 tablets on the first day of treatment. The maximum study duration for each subject is about 12-14 weeks and includes a screening period of 2-4 weeks, a treatment period of 6 weeks, and a follow-up period of 4 weeks. The study parameters are summarized in Table 10.

TABLE 10

| | |
|---|---|
| Study Title: | A Randomized, Double-Blind, Placebo-Controlled Study of |
| | Serlopitant in Subjects with Chronic Pruritus and Atopic |
| | Dermatitis |
| Development Phase: | Phase II |
| Study Objectives: | Primary objective: Assess the efficacy of serlopitant in treating |
| | pruritus in adults with a history of AD |
| | Secondary objective: Assess the safety and tolerability of |
| | repeated oral doses of serlopitant in the subjects |
| Study Design: | Randomized, double-blind, placebo-controlled study |
| Sample Size: | At least about 120 subjects are randomized to receive |
| | serlopitant or placebo, with at least about 60 subjects in each of |
| | the serlopitant and placebo groups. |
| Study Population: | Male and female adults 18-65 years old with a history of atopic |
| | dermatitis (AD) who have pruritus of at least 6-week duration |
| | despite treatment with standard-of-care antipruritic therapies, |
| | and who have a worst itch Numeric Rating Scale (WI-NRS) |
| | score ≥7 at the initial screening visit and an average weekly |
| | WI-NRS score ≥6 for the last 2 weeks of the screening period |
| Investigational Product: | Oral daily tablet of serlopitant |
| Dosage and Frequency | Loading dose of three 5-mg tablets of serlopitant or placebo on |
| | Day 1, followed by one 5-mg tablet of serlopitant or placebo |
| | once daily by mouth for 6 weeks |
| Reference Product: | None |
| Control Product: | Matching placebo once daily for 6 weeks |
| Efficacy Evaluation Criteria: | Primary efficacy endpoint: change in WI-NRS score from |
| | Baseline/Day I to Week 6 |
| | Secondary efficacy endpoints: |
| | change in average itch NRS (AI-NRS) score from Baseline to |
| | Week 6 |
| | proportion of subjects who achieve ≥30% improvement in |
| | WI-NRS score from Baseline to Week 6 |
| | proportion of subjects who achieve ≥30% improvement in |
| | AI-NRS score from Baseline to Week 6 |
| | change in Pruritus-Specific Quality of Life (ItchyQoL) and |
| | Dermatology Life Quality index (DLQI) from Baseline to |
| | Week 6 |
| | change in Patient and Physician Global Assessments from |
| | Baseline to Week 6 |
| Safety Evaluation Criteria: | Safety is assessed by adverse events, serious adverse events, |
| | electrocardiograms, vital signs, abbreviated physical |
| | examinations, and blood and urine laboratory tests. |
| Statistical Methods: | The difference in the primary efficacy endpoint between |
| | treatment groups is assessed using a t-test or a Cochran Mantel |
| | Haenxael (CMH) test controlling for stratification factors. |
| | The secondary efficacy endpoints are summarized with |
| | descriptive statistics by treatment group, and treatment |
| | differences and associated 95% confidence intervals are |
| | produced, or are evaluated using an analysis of variance |
| | (ANOVA) model. |
| Study Sites: | Multicenter |

Other primary efficacy endpoints can also be used, including without limitation the WI-NRS and AI-NRS 4-point responder rates at Week 6. Moreover, other secondary efficacy endpoints can also be used, including without limitation the WI-NRS and AI-NRS 4-point responder rates at the midpoint of the treatment period (Week 3), the WI-NRS and AI-NRS 3-point responder rates at Weeks 3 and 6, the change in WI-NRS and AI-NRS from Baseline to Weeks 2 and 4, the change in 5-D Pruritus Scale from Baseline to Week 6, the change in Static Patient Global Assessment of Itch Severity (sPGA) from Baseline to Week 6, the change in Patient Global Impression of Change in Itch Severity (PGIC) from Baseline to Week 6, and the change in the number of nighttime scratching events per hour from Baseline to Week 6.

Additional or different clinical trials according to a similar study design can be conducted to study, e.g., different dosages (e.g., about 1 mg) or different modes of administration (e.g., dermal or transdermal) of serlopitant, or different lengths of treatment (e.g., about 8 weeks) with serlopitant, or to differentiate between optimal doses or dosing schedules. Furthermore, the efficacy of serlopitant in specific subject populations, such as toddlers (e.g, about 1-3 yea's of age), children (e.g., about 4-10 or 4-12 years of age, which may also include toddlers), adolescents (e.g., about 10-17 or 12-17 years of age), and the elderly (e.g., about 65-80 years of age), and in treating pruritus associated with a different medical condition (e.g., psoriasis [e.g., plaque psoriasis], urticaria [e.g., chronic idiopathic urticaria], CTCL [e.g., mycosis fungoides], epidermolysis bullosa [e.g., EB simplex], a burn [e.g., a thermal burn, a second-degree burn or a third-degree burn, or a moderate burn or a major burn], or a hepato-biliary disease [e.g., a cholestatic disorder such as cholestasis or primary biliary cirrhosis]), can be determined in additional or different clinical trials conducted in a similar fashion.

It is understood that, while particular embodiments have been illustrated and described, various modifications may be made thereto and are contemplated herein. It is also understood that the disclosure is not limited by the specific examples provided herein. The description and illustration of embodiments and examples of the disclosure herein are not intended to be construed in a limiting sense. It is further understood that all aspects of the disclosure are not limited to the specific depictions, configurations or relative proportions set forth herein, which may depend upon a variety of conditions and variables. Various modifications and variations in form and detail of the embodiments and examples of the disclosure will be apparent to a person skilled in the art. It is therefore contemplated that the disclosure also covers any and all such modifications, variations and equivalents.

What is claimed is:

1. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist selected from aprepitant, fosaprepitant, netupitant, orvepitant, rolapitant, tradipitant, vestipitant, DNK-333, SCH-900978, and pharmaceutically acceptable salts thereof, wherein:
the NK-1 antagonist is not aprepitant for the treatment of pruritus associated with atopic dermatitis or prurigo nodularis;
the NK-1 antagonist is not orvepitant for the treatment of pruritus associated with a burn; and
the NK-1 antagonist is not tradipitant for the treatment of pruritus associated with atopic dermatitis.

2. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist and a therapeutically effective amount of an H₄ antihistamine.

3. The method of claim 2, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIT 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

4. The method of claim 2 or 3, wherein the NK-1 antagonist is serlopitant or a pharmaceutically acceptable salt thereof.

5. The method of any one of claims 2 to 4, wherein the H₄ antihistamine is selected from clobenpropit, thioperamide, A943931, A987306, JNJ-7777120, VUF-6002, ZPL-389, and pharmaceutically acceptable salts thereof.

6. The method of claim 5, wherein the H₄ antihistamine is ZPL-389 or a pharmaceutically acceptable salt thereof.

7. The method of any one of claims 2 to 6, wherein the pruritus is associated with dermatitis/eczema (e.g., atopic dermatitis) or psoriasis (e.g., plaque psoriasis).

8. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist and a therapeutically effective amount of a kappa-opioid receptor agonist.

9. The method of claim 8, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIT 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

10. The method of claim 8 or 9, wherein the NK-1 antagonist is serlopitant or a pharmaceutically acceptable salt thereof.

11. The method of any one of claims 8 to 10, wherein the kappa-opioid receptor agonist is selected from asimadoline, bremazocine, butorphanol (a mu antagonist and kappa agonist), difelikefalin (CR845), dynorphin, enadoline, ketazocine, nalbuphine (a mu antagonist and kappa agonist), nalfurafine, salvinorin A, 2-methoxymethyl salvinorin B, 2-ethoxy methyl salvinorin B, 2-fluoroethoxymethyl salvinorin B, spiradoline, tifluadom, BRL-52537, FE 200665, GR-89696, HZ-2, ICI-199,441, ICI-204,448, LPK-26, SA-14867, U-50488, U-69,593, and pharmaceutically acceptable salts thereof.

12. The method of claim 11, wherein the kappa-opioid receptor agonist is asimadoline, butorphanol, difelikefalin (CR845), nalbuphine or nalfurafine, or a pharmaceutically acceptable salt thereof.

13. The method of any one of claims 8 to 12, wherein the pruritus is associated with dermatitis/eczema (e.g., atopic dermatitis), prurigo (e.g., porrigo nodularis), or a hepato-biliary disease (e.g., a cholestatic disorder such as cholestasis or primary biliary cirrhosis).

14. The method of any one of claims 8 to 13, wherein the kappa-opioid receptor agonist is nalbuphine or a pharmaceutically acceptable salt thereof (e.g., Nalbuphine ER), and the pruritus is associated with prurigo (e.g., prurigo nodularis).

15. A method of treating pruritus associated with dermatitis/eczema, psoriasis, prurigo, urticaria, cutaneous T-cell lymphoma (CTCL), epidermolysis bullosa, a burn or a hepato-biliary disease, comprising administering to a subject in need of treatment a therapeutically effective amount of a neurokinin-1 (NK-1) antagonist and a therapeutically effective amount of a mu-opioid receptor antagonist, wherein the NK-1 antagonist is not serlopitant.

16. The method of claim 15, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIT 1149 CL, CGP-4982:3, CJ-17493, CP-96345, CP-99994, CP-122721, DNK-333, FK-224, FK-888, GR-205171, GSK-424887, HSP-1.17, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

17. The method of claim 15 or 16, wherein the mu-opioid receptor antagonist is selected from alvimopan, axelopran, bevenopran, butorphanol (a mu antagonist and kappa agonist), cyprodime, eptazocine, levallorphan (lorfan or naloxiphan), methylnaltrexone, naldemedine, nalmefene, nalbuphine (a mu antagonist and kappa agonist), nalodeine, nalorphine (lethidrone or nalline), naloxegol, naloxone, naloxol, naltrexone, 6β-naltrexol, samidorphan, SK-1405, and pharmaceutically acceptable salts thereof.

18. The method of claim 17, wherein the mu-opioid receptor antagonist is butorphanol, nalmefene, naloxone, naltrexone or SK-1405, or a pharmaceutically acceptable salt thereof.

19. The method of any one of claims 15 to 18, wherein the pruritus is associated with dermatitis/eczema (e.g., atopic dermatitis), prurigo (e.g., prurigo nodularis), CTCL (e.g., mycosis fungoides), a burn, or a hepato-biliary disease (e.g., a cholestatic disorder such as cholestasis or primary biliary cirrhosis).

20. A method of treating pruritus associated with dermatitis/eczema, psoriasis, prurigo, urticaria, cutaneous T-cell lymphoma (CTCL), epidermolysis bullosa, a burn or a hepato-biliary disease, comprising administering to a subject in need of treatment a therapeutically effective amount of a neurokinin-1 (NK-1) antagonist and a therapeutically effective amount of an antidepressant, wherein the NK-1 antagonist is not serlopitant.

21. The method of claim 20, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, tradipitant, vestipitant, vofopitant, hydroxyphenyl propamidobenzoic acid, maltooligosaccharides (e.g., maltotetraose and maltopentaose), spantides (e.g., spantide 1 and 11), AV-608, AV-818, AZD-2624, HUE 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-11.6031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

22. The method of claim 20 or 21, wherein the antidepressant is selected from tricyclic antidepressants (e.g., amitriptyline, amitriptylinoxide, amoxapine, dosulepin [dothiepin], doxepin, cidoxepin and melitracen), tetracyclic antidepressants (e.g., amoxapine, maprotiline, mazindol, mianserin, mirtazapine, esmirtazapine and setiptiline), selective serotonin reuptake inhibitors (SSRIs, e.g., citalopram, dapoxetine, escitalopram, fluoxetine, fluvoxamine, paroxetine and sertraline), serotonin-norepinephrine reuptake inhibitors (SNRIs, e.g., bicifadine, doxepin, cidoxepin, duloxetine, milnacipran, levomilnacipran, sibutramine, venlafaxine, desvenlafaxine and SEP-227162), inhibitors of monoamine oxidases (e.g., selective MAO-A inhibitors [e.g., bifemelane, moclobemide, pirlindole {pirazidol} and toloxatone], selective MAO-B inhibitors [e.g., rasagiline and selegiline], and non-selective MAO-A/MAO-B inhibitors [e.g., hydracarbazine, isocarboxazid, nialamide, phenelzine and tranylcypromine]), and pharmaceutically acceptable salts and combinations thereof.

23. The method of claim 22, wherein the antidepressant is or comprises amitriptyline, doxepin, cidoxepin, mirtazapine, esmirtazapine, fluvoxamine or paroxetine, or a pharmaceutically acceptable salt or any combination thereof.

24. The method of any one of claims 20 to 23, wherein the pruritus is associated with dermatitis/eczema (e.g., atopic dermatitis), prurigo (e.g., prurigo nodularis), CTCL (e.g., mycosis fungoides), epidermolysis bullosa (e.g., epidermolysis bullosa simplex), or a hepato-biliary disease (e.g., a cholestatic disorder such as cholestasis or primary biliary cirrhosis).

25. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist and a therapeutically effective amount of an inhibitor of a pro-inflammatory cytokine or a receptor therefor.

26. The method of claim 25, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, AZD-2624, BIT 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

27. The method of claim 25 or 26, wherein the NK-1 antagonist is serlopitant or a pharmaceutically acceptable salt thereof.

28. The method of any one of claims 25 to 27, wherein the inhibitor of a pro-inflammatory cytokine or a receptor therefor is selected from inhibitors of tumor necrosis factor-alpha (TNF-α) (e.g., adalimumab, certolizumab pegol, golimumab, infliximab, etanercept, bupropion and ART-620, inhibitors of interleukin-2 (IL-2) or receptor therefor (IL-2R) (e.g., basiliximab and daclizumab), inhibitors of IL-4 or IL-4R (e.g., dupilumab), inhibitors of IL-12 (e.g., briakinumab and ustekinumab) or IL-12R, inhibitors of IL-17 (e.g., ixekizumab and secukinumab) or IL-17R (e.g., brodalumab), inhibitors of IL-22 (e.g., fezakimimab) or IL-22R, inhibitors of IL-23 (e.g., briakinumab, guselkumab risankizumab, tildrakizumab [SCH-900222], ustekinumab and BI-6550661 or IL-23R, inhibitors of IL-31 or IL-31R (e.g., nernolizumab), and pharmaceutically acceptable salts and combinations thereof.

29. The method of any one of claims 25 to 28, wherein the pruritus is associated with dermatitis/eczema (e.g., atopic dermatitis), psoriasis (e.g., plaque psoriasis), or prurigo (e.g., prurigo nodularis).

30. The method of any one of claims 25 to 29, wherein the inhibitor of a pro-inflammatory cytokine or a receptor therefor is or comprises an inhibitor of IL-2 or IL-2R (e.g., basiliximab or daclizumab), an inhibitor of IL-4 or IL-4R (e.g., dupilumab), or an inhibitor of IL-31 or IL-31R (e.g., nemolizumab), or a pharmaceutically acceptable salt or any combination thereof, and the pruritus is associated with dermatitis/eczema (e.g., atopic dermatitis).

31. The method of any one of claims 25 to 29, wherein the inhibitor of a pro-inflammatory cytokine or a receptor therefor is or comprises a TNF-α, inhibitor (e.g., adalimumab, certolizumab pegol, infliximab or etanercept), an inhibitor of IL-12 (e.g., ustekinumab) or IL-12R, an inhibitor of IL-17 (e.g., ixekizumab or secukinumab) or IL-17R (e.g., brodalumab), an inhibitor of IL-22 (e.g., fezakinumab) or IL-22R, or an inhibitor of IL-23 guselkumab, risankizumab, tildrakizumab or ustekinumab) or IL-23R, or a pharmaceutically acceptable salt or any combination thereof, and the pruritus is associated with psoriasis (e.g., plaque psoriasis).

32. The method of any one of claims 25 to 29, wherein the inhibitor of a pro-inflammatory cytokine or a receptor therefor is or comprises an inhibitor of IL-31 or IL-31R (e.g., nemolizumab or a pharmaceutically acceptable salt thereof), and the pruritus is associated with prurigo (e.g., prurigo nodularis).

33. A method of treating pruritus associated with dermatitis/eczema, psoriasis, prurigo, urticaria, cutaneous T-cell lymphoma, epidermolysis bullosa, a burn or a hepato-biliary disease, comprising administering to a subject in need of treatment a therapeutically effective amount of a neurokinin-1 (NK-1) antagonist and a therapeutically effective amount of a phosphodiesterase-4 (PDE4) inhibitor, wherein the NK-1 antagonist is not serlopitant for the treatment of pruritus associated with psoriasis.

34. The method of claim 33, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidohenzoic acid, maltooligosaccharides (e.g., maltotetraose and maltopentaose), spantides (e.g., spantide 1 and 11), AV-608, AV-818, AZD-2624, BITE 1149 CL, CGP-4982:3, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

35. The method of claim 33 or 34, wherein the NK-1 antagonist is serlopitant or a pharmaceutically acceptable salt thereof.

36. The method of any one of claims 33 to 35, wherein the PDE4 inhibitor is selected from apremilast, cilomilast, ibudilast, piclamilast, roflumilast, crisaborole, diazepam, luteolin, mesembrenone, rolipram, AN2728, E6005, and pharmaceutically acceptable salts thereof.

37. The method of claim 36, wherein the PDE4 inhibitor is apremilast or crisaborole or a pharmaceutically acceptable salt thereof.

38. The method of any one of claims 33 to 37, wherein the pruritus is associated with dermatitis/eczema (e.g., atopic dermatitis) or psoriasis g plaque psoriasis).

39. The method of any one of claims 33 to 38, wherein the PDE4 inhibitor is apremilast or a pharmaceutically acceptable salt thereof, and the pruritus is associated with psoriasis (e.g., plaque psoriasis).

40. A method of treating pruritus associated with a hepato-biliary disease, comprising administering to a subject in need of treatment a therapeutically effective amount of a neurokinin-1 (NK-1) antagonist and a therapeutically effective amount of a farnesoid X receptor (FXR) agonist.

41. The method of claim 40, wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidohenzoic acid, maltooligosaccharides (e.g., maltotetraose and maltopentaose), spantides (e.g., spantide I and II) AV-608, AV-818, AZD-2624, BITE 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

42. The method of claim 40 or 41, wherein the NK-1 antagonist is serlopitant or a pharmaceutically acceptable salt thereof.

43. The method of any one of claims 40 to 42, wherein FXR agonist is selected from cafestol, chenodeoxycholic acid, obeticholic acid, fexaramine, and pharmaceutically acceptable salts thereof.

44. The method of claim 43, wherein the FXR agonist is obeticholic acid or a pharmaceutically acceptable salt thereof.

45. The method of any one of claims 40 to 44, wherein the pruritus is associated with a cholestatic disorder (e.g., cholestasis or primary biliary cirrhosis [primary biliary cholangitis]).

46. The method of claim 45, further comprising administering a cholesterol absorption-reducing or gallstone-dissolving agent (e.g., ursodeoxycholic acid [ursodiol] or chenodeoxycholic acid).

47. A method of treating pruritus associated with dermatitis/eczema, psoriasis, prurigo, urticaria, cutaneous T-cell lymphoma (CTCL), epidermolysis bullosa, a burn or a hepato-biliary disease, comprising administering to a subject in need of treatment a therapeutically effective amount of a neurokinin-1 (NK-1) antagonist and a therapeutically effective amount of an additional therapeutic agent, wherein:
the additional therapeutic agent is or comprises asimadoline, difelikefalin (CR845), nalbuphine, nalfurafine, SK-1405, 8-777469, LPL-389, CT327, apremilast, crisaborole, EBI-005, dupilumab, nemolizumab, NST-141 or SD-101, or a pharmaceutically acceptable salt or any combination thereof;
the NK-1 antagonist is not serlopitant for use in combination with CT327 to treat pruritus associated with atopic dermatitis, psoriasis or CTCL; and
the NK-1 antagonist is not serlopitant for use in combination with apremilast or crisahorole to treat pruritus associated with psoriasis.

48. The method of claim 47, wherein the NK-1 antagonist is not serlopitant for use in combination with nalbuphine.

49. The method of claim 47, wherein the NK-1 antagonist is not serlopitant for use in combination with SK-1405.

50. The method of any one of claims 47 to 49 wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidobenzoic acid, maltooligosaccharides (e.g., maltotetraose and maltopentaose), spantides (e.g., spantide I and II), AV-608, AV-818, ALD-2624, BIIF 1149 CL, CGP-49823, CJ-17493, CP-96345, CP-99994, CP-122721, DNK-333, FK-224, FK-888, GR-205171, GSK-424887, HSP-117, KRP-1103, L-703606, L-73:3060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

51. The method of claim 50, wherein the NK-1 antagonist is serlopitant or a pharmaceutically acceptable salt thereof.

52. A method of preventing pruritus, comprising administering to a subject a therapeutically effective amount of a neurokinin-1 (NK-1) antagonist prior to development of pruritus.

53. The method of claim 52 wherein the NK-1 antagonist is selected from aprepitant, fosaprepitant, befetupitant, casopitant, dapitant, ezlopitant, lanepitant, maropitant, netupitant, nolpitantium, orvepitant, rolapitant, serlopitant, tradipitant, vestipitant, vofopitant, hydroxyphenyl propamidohenzoic acid, maltooligosaccharides (e.g., maltotetraose and maltopentaose), spantides (e.g., spantide I and II), AV-608, AV-818, AZD-2624, BIIF 1149 CL, CGP-49823, CJ-17493, CP-96345, CP-99994, CP-122721, DNK-333, FK-224, FK-888, GR-205171, GSK-424887, HSP-117, KRP-103, L-703606, L-733060, L-736281, L-759274, L-760735, LY-686017, M516102, MDL-105212, NKP-608, R-116031, R-116301, RP-67580, SCH-206272, SCH-388714, SCH-900978, SLV-317, SSR-240600, T-2328, TA-5538, TAK-637, TKA-731, ZD-4974, ZD-6021, and pharmaceutically acceptable salts thereof.

54. The method of claim 52 or 53, wherein the NK-1 antagonist is serlopitant or a pharmaceutically acceptable salt thereof.

55. The method of any one of claims 52 to 54, wherein the pruritus is acute pruritus.

56. The method of any one of the preceding claims, further comprising administering one or more additional antipruritic or therapeutic agents.
